(19) United States
(12) Patent Application Publication
(10) Pub. No.: US 2020/0377958 A1
(43) Pub. Date: Dec. 3, 2020

(54) BIOMARKERS AND METHODS FOR TREATMENT WITH NAE INHIBITORS

(71) Applicant: Millennium Pharmaceuticals, Inc., Cambridge, MA (US)

(72) Inventors: Ajeeta Buddhadev DASH, Somerville, MA (US); Andrew S. KRUEGER, Amesbury, MA (US)

(21) Appl. No.: 16/766,397

(22) PCT Filed: Nov. 30, 2018
(86) PCT No.: PCT/US2018/063446

§ 371 Date: May 22, 2020

Related U.S. Application Data
(60) Provisional application No. 62/593,686, filed on Dec. 1, 2017.

(51) Int. Cl. C12Q 1/6886

(52) U.S. Cl. CPC C12Q 1/6886; C12Q 2600/106; G01N 2800/60; C12Q 2600/156; C12Q 2600/158; C12Q 2600/154

(57) ABSTRACT

Disclosed herein are markers whose mutational status is associated with sensitivity to treatment with NAE inhibitors. Mutational status is determined by measurement of characteristics of markers corresponding to the marker genes. Compositions and methods are provided to assess markers of marker genes to predict response to NAE inhibition treatment.

### RELATED APPLICATIONS

This application claims priority to U.S. Provisional Application No. 62/593,686 filed on Dec. 1, 2017. The entire contents of the foregoing application are incorporated herein by reference.

### SEQUENCE LISTING

This application contains a Sequence Listing which is submitted herewith in electronically readable format. The Sequence Listing file was created on Nov. 30, 2018, is named “3817_041_b_ST25.txt,” and its size is 138 kb (141,774 bytes). The entire contents of the Sequence Listing in the sequencelisting.txt file are incorporated herein by this reference.

### BACKGROUND

Cells become cancerous when their genotype or phenotype alters in a way that there is uncontrolled growth that is not subject to the confines of the normal tissue environment. Genetic alterations may be seen when one or more genes is mutated, amplified, deleted, overexpressed or underexpressed. These alterations may result in changes to the protein corresponding to the gene. Additionally, chromosome portions can be lost or moved from one location to another. Some cancers have characteristic patterns by which genotypes or phenotypes are altered. Genetic alterations can facilitate tumor progression, tumor growth rate or whether a tumor will metastasize. Some genetic alterations can affect whether a tumor cell will respond to therapy.

Hematological cancers of the hematopoietic and lymphoid tissues have a variety of treatment options, such as chemotherapy, radiation therapy, immunotherapy and stem cell transplantation. Depending on the origin, location or severity of the cancer, the chemotherapy might comprise treatment with antimetabolites, antimitotics, alkylating agents, histone deacetylase inhibitors, hypomethylating agents, proteasome inhibitors, kinase inhibitors, immunomodulators, and/or other agents, such as new agents acting in recently studied pathways.

One set of recently studied pathways relates to E1 enzyme activity. Ubiquitin and other ubiquitin-like molecules (ubls) are activated by a specific enzyme (an E1 enzyme, an ATP-dependent activating enzyme) which catalyzes the formation of an acyl-adenylate intermediate with the C-terminal glycine of the ubl. The activated ubl is then transferred to a catalytic cysteine residue within the E1 enzyme through formation of a thioester bond intermediate. After additional enzymatic steps, the ubl is then conjugated to the target protein, through isopeptide bond formation with the amino group of a lysine side chain in the target protein. Inhibiting E1 enzymatic activity means reducing the ability of an E1 enzyme to activate ubiquitin like (ubl) conjugation to a substrate peptide or protein (e.g., ubiquitination, neddylation, sumoylation). The ubl named Neural precursor cell-Expressed Developmentally Downregulated 8 (NEDD8) is activated by the heterodimer NEDD8-activating enzyme (NAE, also known as APPBP1-UBA3, UBEIC (ubiquitin-activating enzyme E1C)) and is transferred to one of two E2 conjugating enzymes (ubiquitin carrier protein 12 (UBC12) and UBC17), ultimately resulting in ligation of NEDD8 to cullin proteins by the cullin-RING subtype of ubiquitin ligases. A function of neddylation is the activation of cullin-based ubiquitin ligases involved in the turnover of many cell cycle and cell signaling proteins, including p27 and I-κB. See Pan et al., Oncogene 23:1985-97 (2004). Inhibition of NAE can disrupt cullin-RING ligase-mediated protein turnover and can lead to apoptotic death in cells, e.g., tumor cells or cells of a pathogenic organism, e.g. a parasite. See Soucy et al., (2010) Genes & Cancer 1:708-716.

Some patients being treated with chemotherapeutic agents respond to one agent better than another. Valuable time in a patient's treatment program can be lost pursuing therapy with an agent which eventually is proven ineffective for that patient or leads to treatment resistance of the cancer. As cancer is a progressive, deadly disease, many patients cannot afford the time for trial-and-error choices of therapeutic regimens. Expedient and accurate treatment based on analytical results for patients likely to have favorable therapeutic outcomes lead to effective management of the cancer.

### SUMMARY

The present disclosure relates to methods and kits for treatment of cancer, e.g., a hematological cancer in patients, e.g., human patients, who are characterized by marker analysis for favorable outcome of treatment. Marker analysis can include measurement of the amount, presence or changes of markers provided herein. The markers are predictive of whether there will be a favorable outcome (e.g., good response, long time-to-progression, negative minimal residual disease, long progression-free survival and/or long term survival) after treatment with a regimen comprising a NEDD8-activating enzyme (NAE) inhibitor, such as pevonedistat or a pharmaceutically acceptable salt thereof. A treatment regimen comprising pevonedistat treatment can further comprise treatment with one or more additional agent, such as a hypomethylating agent, e.g., azacitidine, or a pharmaceutically acceptable salt thereof. Testing a biological sample, e.g., a sample obtained from a patient, e.g., a sample comprising tumor cells or contents or products thereof, e.g., in vitro, to detect or measure the presence, amounts or changes of genetic or phenotypic markers, e.g., the mutational status of at least one marker gene, identifies particular patients who are expected to have a favorable outcome with treatment, e.g., with a regimen comprising an NAE inhibitor, such as pevonedistat or a pharmaceutically acceptable salt thereof, and whose disease may be managed by standard or less aggressive treatment with a regimen comprising an NAE inhibitor, such as pevonedistat or a pharmaceutically acceptable salt thereof, as well as those patients who are expected have an unfavorable outcome with the treatment and may require an alternative treatment to, a combination of treatments and/or more aggressive treatment, e.g., a more frequent dosing regimen or higher dose with the NAE inhibitor to ensure a favorable outcome and/or successful management of the disease.

In one aspect, the invention provides compositions, such as kits, or methods useful in detecting or measuring characteristics, e.g., amounts, presence or changes, of the markers in a biological sample, e.g., a sample obtained from a cancer patient, e.g., a human patient. Such compositions and methods can determine the mutational status of marker genes described herein.

In another aspect, the invention provides disease, e.g., cancer, management strategies. In the foregoing aspects, the characteristic, e.g., size, sequence, composition or amount of marker, e.g., nucleic acid or protein, in a biological sample comprising tumor cells, e.g., hematological tumor cells, or contents or products thereof is measured. In one embodiment, the cancer is a hematological cancer, such as leukemia, lymphoma or myeloma. In some embodiments, the hematological cancer is acute myelogenous leukemia (AML), myelodysplastic syndrome (MDS) or chronic myelomonocytic leukemia (CMML). In some embodiments, the MDS is high risk MDS. In some embodiments, the AML is low-blast acute AML.

In various embodiments, the marker characteristic, e.g., size, sequence, composition or amount of DNA, the size, sequence, composition or amount of RNA and/or the size, sequence, composition or amount of protein corresponding to a marker gene, either wild type or with one or more genotypic or phenotypic alteration, e.g., a polymorphism or mutation, e.g., somatic mutation, described herein is measured. In some embodiments, the measurement indicates that a gene is underexpressed or overexpressed. In some embodiments, the measurement indicates that a gene is amplified, deleted or translocated. In some embodiments, the measurement indicates that the gene is misexpressed or the altered gene product alters a signaling or functional pathway contributing to the cancer state. Disease management strategy is undertaken when assay results reveal information about a marker gene or marker genes, e.g., whether a gene is altered, or not, the identity of the alteration, and/or whether the RNA or protein amount of an altered marker gene or marker genes indicates favorable outcome to therapy comprising NAE inhibition, e.g., pevonedistat therapy. By examining the marker characteristic, e.g., size, sequence, composition or amount of one or more of the identified markers, it is therefore possible to eliminate ineffective or inappropriate therapeutic agents or regimens. Thus, one can undertake a therapeutic regimen which is likely to benefit a particular patient or type of patient, e.g., whether a particular regimen should be started or avoided, continued, discontinued or altered. Such analyses can be made on a patient-by-patient basis, e.g., identifying and/or selecting for treatment a cancer patient who is expected to demonstrate a favorable outcome upon administration of a therapeutic regimen, e.g., a therapeutic regimen comprising an NAE inhibitor, such as pevonedistat or a pharmaceutically acceptable salt thereof.

A marker gene useful for analysis in methods or kits described herein is a cancer marker gene, e.g., a marker gene selected from the group consisting of ten-eleven translocation methylcytosine dioxygenase 2 (TET2), runt-related transcription factor 1 (RUNX1), neuroblastoma RAS viral (v-ras) oncogene homolog (NRAS), v-Ki-ras2 Kirsten rat sarcoma viral oncogene homolog (KRAS), DNA methyltransferase 3 alpha (DNMT3A), tumor protein p53 (TP53), isocitrate dehydrogenase (NADP(+)) 2, mitochondrial (IDH2), enhancer of zeste 2 polycomb repressive complex 2 subunit (EZH2), isocitrate dehydrogenase (NADPH(+)) 1, cytosolic (IDH1), nucleophosmin 1 (NPM1), plant homeodomain-like finger protein 6 (PHF6), and additional sex combs-like 1 (ASXL1). A result with mutational status, such as wild type status or alterations whose presence in DNA or whose effects, e.g., on marker RNA and/or protein characteristics, e.g., amounts, size, sequence or composition, provides a selection, treatment or disease management of cancer patients. In some embodiments, a gene or a mutant or genetically altered form thereof is useful, has a DNA, an RNA and/or protein characteristic, e.g., size, sequence, composition or amount, e.g., in a biological sample comprising tumor cells, e.g., hematological tumor cells, or contents or products thereof, if it is different than a normal DNA, RNA and/or protein. Described herein are examples of modifications of these genes, referred to as “marker genes” whose analysis can provide such results.

In some embodiments, these methods include measuring, determining, receiving, storing or transmitting results about the marker characteristic, e.g., size, sequence, composition or amount of one or more markers or alteration of marker gene(s) in a patient's cancer (e.g., a patient's cancer cells, e.g., hematological cancer cells (e.g., tumor cells), or contents or products thereof), optionally comparing that to the characteristic, e.g., size, sequence, composition or amount of a normal, control or reference marker, and in a further embodiment, when the result from the sample corresponds to a favorable outcome of a treatment regimen, e.g., comprising an NAE inhibitor, such as pevonedistat or a pharmaceutically acceptable salt thereof treatment regimen, taking a financial step, such as paying for the treatment or issuing an insurance policy to the patient.

In some embodiments therapeutic methods further include the step of beginning, continuing, or commencing a therapy accordingly where the presence of an alteration in a marker gene or the characteristic, e.g., size, sequence, composition or amount of a patient's marker or markers, e.g., in a first and/or subsequent biological sample, e.g., a sample obtained from the patient, e.g., comprising tumor cells, e.g., hematological tumor cells, or contents or products thereof, indicates that the patient is expected to have a favorable outcome with the therapy, e.g., the NAE inhibitor, such as a pevonedistat or a pharmaceutically acceptable salt thereof therapeutic regimen. In addition, the methods include therapeutic methods which further include the step of stopping, discontinuing, altering or halting a therapy accordingly where the presence of an alteration in a marker gene or the characteristic, e.g., size, sequence, composition or amount of a patient's marker, e.g., in a second sample obtained from the patient, indicates that the patient is expected to demonstrate an unfavorable outcome with the treatment, e.g., with the NAE inhibitor, such as a pevonedistat or a pharmaceutically acceptable salt thereof regimen, e.g., as compared to a patient identified as having a favorable outcome receiving the same therapeutic regimen. In another aspect, methods are provided for analysis and treatment of a patient not yet being treated with a therapy, e.g., an NAE inhibitor, such as a pevonedistat or a pharmaceutically acceptable salt thereof therapy and identification and report of predicted outcome of the treatment based upon the presence of an alteration in a marker gene or characteristic, e.g., size, sequence, composition or amount of one or more of a patient's marker described herein. Such methods can include not being treated with the therapy, being treated with the therapy, being treated with the therapy in combination with one more additional therapies, or being treated with a more aggressive dosing and/or administration regimen, e.g., a more frequent dosing regimen or higher dose with the NAE inhibitor therapy, as compared to the dosing and/or administration regimen of a patient identified as having a favorable outcome to standard NAE inhibitor, such as a pevonedistat or a pharmaceutically acceptable salt thereof therapy. Thus, the provided methods of the invention can eliminate ineffective or inappropriate use of therapy, e.g., NAE inhibitor, such as pevonedistat or a pharmaceutically acceptable salt thereof therapy regimens.

Additional methods include methods to identify new therapeutic combinations. Such methods include methods to identify an agent as useful in combination with an NAE inhibitor, such as pevonedistat or a pharmaceutically acceptable salt thereof, for treating a cancer, e.g., a hematological cancer (e.g., myeloma, leukemias, lymphoma, etc.), based on its ability to affect the presence of a genotypic or phenotypic alteration in a marker gene or characteristic, e.g., size, sequence, composition or amount of a marker or markers of the invention. For example, an agent which in combination with an NAE inhibitor, such as pevonedistat or a pharmaceutically acceptable salt thereof, decreases or increases the presence of an alteration in a marker gene or characteristic, e.g., size, sequence, composition or amount of a marker or markers provided herein in a manner that indicates favorable outcome of a patient having a cancer, e.g., a hematological cancer would be a candidate agent for the combination. Alternatively, an agent which in combination with an NAE inhibitor, such as pevonedistat or a pharmaceutically acceptable salt thereof, is able to decrease the viability of a tumor cell comprising a marker indicative of a favorable outcome would be a candidate agent for the combination.

The present invention is also directed to methods of treating a cancer patient, e.g., a human patient, with a therapeutic regimen, e.g., with an NAE inhibitor, such as a pevonedistat or a pharmaceutically acceptable salt thereof therapy regimen (e.g., alone, or in combination with an additional agent such as a chemotherapeutic agent, e.g., a proteasome inhibitor, an alkylating agent, hypomethylating agent, an antibiotic or an antimetabolite), which includes the step of selecting for treatment a patient whose marker characteristic, e.g., size, sequence, composition or amount, indicates that the patient is expected to have a favorable outcome with the therapeutic regimen, and treating the patient with the therapy, e.g., NAE inhibition, such as a pevonedistat or a pharmaceutically acceptable salt thereof therapy. In some embodiments, the method can include the step of administering therapy to a patient whose cancer is characterized by marker characteristic, e.g., size, sequence, composition or amount or amounts indicative of a favorable outcome. In some embodiments a favorable outcome in the methods includes the use of pevonedistat or a pharmaceutically acceptable salt thereof for treating a patient characterized as having tumor cells, e.g., hematological tumor cells, wherein ASXL1 and IDH1 are wild type and wherein at least one marker gene selected from the group consisting of TET2, NRAS, KRAS, DNMT3A, TP53 and RUNX1 has a genetic alteration. In some embodiments a favorable outcome in the methods include the use of pevonedistat or a pharmaceutically acceptable salt thereof for treating a patient characterized as having tumor cells, e.g., hematological tumor cells, wherein ASXL1 and IDH1 are wild type and wherein at least one marker gene selected from the group consisting of TET2, NRAS, KRAS, DNMT3A and RUNX1 has a genetic alteration. In some embodiments, a favorable outcome in the methods further includes the use of a hypomethylating agent, e.g. azacitidine.

Additional methods include a method to pay for the treatment of cancer, e.g. a hematological cancer (e.g., myeloma, leukemias, lymphoma) comprising reviewing, collating or transmitting the analysis result, such as the detection or measurement of a characteristic, e.g., size, sequence, composition or amount, of a patient's marker or marker genes for indication of outcome to a cancer therapy, e.g., an NAE inhibitor, such as a pevonedistat or a pharmaceutically acceptable salt thereof therapy regimen, and paying for treatment with the NAE inhibitor.

The entire contents of all publications, patent applications, patents and other references mentioned herein are incorporated by reference.

Other features and advantages of the invention will be apparent from the following detailed description, drawings and from the claims.

### DRAWINGS

FIG. 1. Heatmap showing mutational status of 11 frequently mutated genes and response data for response-evaluable patients in the MTD cohort. Genetic mutation data for 28 of 52 response-evaluable patients identified using a targeted NGS panel are shown. Each column represents a single patient, and each row represents a single gene. Presence of a mutation in any gene is denoted in red. *Mutation frequency=(# of patients with mutation/# of NGS-evaluable patients)*100. \hat{ }Responders=CR+CRi+PR

FIG. 2. Heatmap showing mutational status of all 38 mutated genes and response data for response-evaluable patients in the MTD cohort. Genetic mutation data identified by targeted NGS for all 38 genes that were found to be mutated in the 28 of 52 response-evaluable patients are shown. Each column represents a single patient, and each row represents a single gene. Presence of a mutation in any gene is denoted as a red box in red. *Mutation frequency=(# of patients with mutation/# of NGS-evaluable patients)*100. \hat{ }Responders=CR+CRi+PR

### DETAILED DESCRIPTION

One of the continued problems with therapy in cancer patients is individual differences in response to therapies. While advances in development of successful cancer therapies progress, only a subset of patients respond to any particular therapy. With the narrow therapeutic index and the toxic potential of many available cancer therapies, such differential responses potentially contribute to patients undergoing unnecessary, ineffective and even potentially harmful therapy regimens. If a designed therapy could be targeted to treat individual patients, such situations could be reduced or even eliminated. Furthermore, targeted designed therapy may provide more focused, successful patient therapy overall. Accordingly, there is a need to identify, select and treat particular cancer patients, e.g., hematological cancer patients, who are expected to have a favorable outcome when administered particular cancer therapies as well as particular cancer patients who may have a favorable outcome using more aggressive and/or alternative cancer therapies, e.g., alternative to previous cancer therapies administered to the patient. It would also be beneficial to provide for the diagnosis, staging, prognosis, and monitoring of cancer patients, including, e.g., hematological cancer patients (e.g., patients suffering from leukemia, lymphoma, myeloma) who would benefit from particular cancer treatment therapies as well as those who would benefit from a more aggressive and/or alternative cancer treatment therapy, e.g., alternative to a cancer therapy or therapies the patient has received, thus resulting in appropriate preventative measures.

The present invention is based, in part, on the mutational status of a marker gene in samples of cancer patients, e.g. hematological cancer patients, who had a favorable outcome of treatment with a regimen comprising an NAE inhibitor, such as pevonedistat or a pharmaceutically acceptable salt thereof. In some embodiments, the regimen comprising an NAE inhibitor further comprises a hypomethylating agent, e.g., pevonedistat or a pharmaceutically acceptable salt in combination with azacitidine. In some embodiments, the marker gene, e.g., the hematological cancer marker gene, is an oncogene homolog, a transcription factor, involved in myelopoiesis, hematopoiesis, epigenetic regulation, gene silencing, chromatin or spliceosome structure or a signaling pathway, e.g., a gene whose encoded protein interacts with a nucleic acid or pathway modulator for tumorigenesis. A protein encoded by a marker gene can have a wild type function in differentiation or cell maturation. Examples of cancer marker genes include TET2, RUNX1, NRAS, KRAS, and DNMT3A. Other examples of cancer marker genes, e.g., for analysis in combination with analysis of one or more of TET2, RUNX1, NRAS, KRAS, and/or DNMT3A marker gene, include TP53, IDH2, EZH2, IDH1, NPM1, PHF6, and ASXL1. Such marker genes can be found with genetic alterations, e.g., mutations or truncations, in hematological cancers, such as AML or MDS. In some embodiments, at least one marker gene, e.g., a hematological cancer marker gene, selected from the group consisting of TET2, RUNX1, NRAS, KRAS, DNMT3A, TP53, IDH2, EZH2, NPM1 and PHF6 has a genetic alteration. Marker genes can be wild type or exhibit genotypic or phenotypic alteration, e.g., a copy number change, a polymorphism or mutation, e.g., somatic mutation, whose presence can affect expression or activity of the encoded gene product. In some embodiments, there can be more than one alteration in a marker gene or more than one marker gene with an alteration in a patient's biological sample, such as a tumor cell, tumor or product, e.g., secretion, from a tumor cell. In additional embodiments, there can be marker gene alterations in cells which have alterations in additional genes, including alterations that can lead to tumorigenesis, but the additional altered genes may not be marker genes as considered herein. In some embodiments, the alteration is an inactivating mutation. In some embodiments, the alteration is an activating mutation. In other embodiments, the alteration affects the expression of the marker gene. In other embodiments, a genotypic alteration can result in an altered interaction of the encoded gene product with a cellular binding partner.

In some embodiments, a combination of marker genes indicates a favorable outcome to treatment with a regimen comprising pevonedistat or a pharmaceutically acceptable salt thereof. In one marker gene combination embodiment, ASXL1 and IDH1 are wild type and either TET2, NRAS, KRAS, DNMT3A, TP53 or RUNX1 is altered in a sample from a patient, e.g., a human patient. For example, a patient with an alteration in TET2, NRAS, KRAS, DNMT3A, TP53 or RUNX1 and no alteration in ASXL1 or IDH1 will respond to a therapeutic regimen comprising pevonedistat or a pharmaceutically acceptable salt, such as pevonedistat or a pharmaceutically acceptable salt plus a hypomethylating agent e.g. azacitidine. In another marker gene combination embodiment, ASXL1 and IDH1 are wild type and one or more of TET2, NRAS, KRAS, DNMT3A, TP53 or RUNX1 is altered in a sample from a patient.

In other embodiments, a favorable outcome to treatment with a regimen comprising pevonedistat or a pharmaceutically acceptable salt thereof is indicated from an altered marker gene combination such as, alterations in KRAS and DNMT3A, alterations in RUNX1 and IDH2, mutations in KRAS and EZH2, mutations in NRAS and EZH2, alterations in TP53 and PHF6, alterations in DNMT3A and NPM1, or alterations in KRAS and NPM1. In some embodiments, a favorable outcome to treatment with a regimen comprising pevonedistat or a pharmaceutically acceptable salt thereof is indicated from any of the above combinations further in combination with wild type ASXL1 and IDH1 marker genes.

In some embodiments, a cancer marker gene is a gene listed in Table 2. In some embodiments, a hematological cancer marker gene is selected from the group consisting of TET2, RUNX1, NRAS, KRAS, DNMT3A, TP53, IDH2, EZH2, IDH1, NPM1, PHF6, and ASXL1. In some embodiments, a solid tumor marker gene is selected from the group consisting of NRAS, KRAS and TP53.

The mutational status result, e.g., identification, detection and/or measurement of the wild type gene or a mutation in a marker gene can be used to determine whether a favorable outcome can be expected by treatment of a tumor, e.g., with a regimen comprising an NAE inhibitor, such as pevonedistat or a pharmaceutically acceptable salt thereof, therapy or whether an alternative therapy to and/or a more aggressive therapy with, e.g., an NAE inhibitor, such as pevonedistat or a pharmaceutically acceptable salt thereof may enhance expected survival time. For example, the compositions and methods provided herein can be used to determine whether a patient is expected to have a favorable outcome to treatment with an NAE inhibitor, such as a pevonedistat or a pharmaceutically acceptable salt thereof therapeutic agent or to an NAE inhibitor, such as a pevonedistat or a pharmaceutically acceptable salt thereof dosing or administration regimen. In general, mutation in differentiation or cell maturation, e.g., gene regulation (oncogene, transcription factor, epigenetic gene silencing) marker genes described herein is associated with sensitivity to or favorable outcome of treatment with a regimen comprising an NAE inhibitor. Examples of marker genes which can function as a cancer marker gene, e.g. a hematological cancer gene, function as an oncogene, a transcription factor, a gene silencer in pathways related to differentiation, cell maturation, such as myelopoiesis or hematopoiesis and whose mutational status is associated with sensitivity to NAE inhibition include TET2, RUNX1, NRAS, KRAS, and/or DNMT3A marker gene, optionally also TP53, IDH2, EZH2, IDH1, NPM1, PHF6, and/or ASXL, such as ASXL1.

Based on these results, the present invention provides, without limitation: 1) methods and compositions for determining whether a treatment regimen comprising an NAE inhibitor, such as pevonedistat or a pharmaceutically acceptable salt thereof will or will not be effective to achieve a favorable outcome and/or manage the cancer; 2) methods and compositions for monitoring the effectiveness of an NAE inhibitor, such as a pevonedistat or a pharmaceutically acceptable salt thereof therapy (alone or in a combination of agents) and dosing and administrations used for the treatment of cancer e.g., a hematological cancer; 3) methods and compositions for treatment of cancer e.g., a hematological cancer comprising administering an NAE inhibitor, such as pevonedistat or a pharmaceutically acceptable salt thereof; 4) methods and compositions for identifying specific combinations of therapeutic agents as well as dosing and administration regimens that are effective for the treatment of cancer, e.g., a hematological cancer in specific patients; and 5) methods and compositions for cancer, e.g., hematological management strategies.

As used herein, the term “NAE inhibitor” refers to an inhibitor of the NEDD8-activating enzyme (NAE) heterodimer. Examples of NAE inhibitors include pevonedistat (formerly known as MLN4924 and TAK-924) or a pharmaceutically acceptable salt thereof. Langston S. et al. U.S. Pat. No. 8,207,177, whose PCT application was published as WO07/092213, WO06084281 and WO2008/019124 (the entire contents of each of the foregoing published patent applications are hereby incorporated by reference), disclose compounds which are effective inhibitors of NAE. In some embodiments, NAE inhibitors do not inhibit, or are very poor at inhibiting, other (non-NAE) E1 enzymes. The compounds are useful for inhibiting NAE activity in vitro and in vivo and are useful for the treatment of disorders of cell proliferation, e.g., cancer, and other disorders associated with NAE activity, such as pathogenic infections and neurodegenerative disorders. One class of compounds described in Langston et al. are 4-substituted ((S, 2S, 4R)-2-hydroxy-4-{7H-pyrrolo[2,3-d]pyrimidin-7-yl}cyclopentyl)methyl sulfamates.

Pevonedistat (((1S,2S,4R)-4-{4-[(1S)-2,3-dihydro-1H-inden-1-ylamino]-7H-pyrrolo[2,3-d]pyrimidin-7-yl}-2-hydroxycyclopentyl)methyl sulfamate) is an NAE-specific E1 inhibitor which disrupts cullin-RING ligase-mediated protein turnover leading to apoptotic death in human tumor cells by perturbation of cellular protein homeostasis (Soucy et al. (2009) Nature 458:732-736). The evaluation of pevonedistat (MLN4924) in cellular and tumor xenograft studies has revealed two distinct mechanisms of action. The first is the induction of DNA re-replication, DNA damage and cell death through MLN4924-mediated dysregulation of the CRL1^{SKP2} and CRL4^{DDB1} substrate Cdt-1 (Milhollen et al. (2011) Cancer Res. 71:3042-3051). It has been shown that p53 status does not impact the induction of DNA re-replication but may make cells more prone to undergo apoptosis or senescence depending on the appropriate genetic background (Milhollen et al. (2011) supra, Lin et al. (2010) Nature 464:374-379 and Lin et al. (2010) Cancer Res. 70:10310-20). The second mechanism is the inhibition of NF-κB pathway activity in NF-κB dependent Diffuse Large B-Cell Lymphomas primarily through dysregulation of CRL1^{βTRCP} mediated turnover of phosphorylated IκBα (Milhollen et al. (2010) Blood 116:1515-1523). In addition, pre-clinical models of Acute Myelogenous Leukemia (AML) are sensitive to pevonedistat inhibition in both cell lines and primary patient blasts through mechanisms related to Cdt-1 dysregulation, NF-κB inhibition and induction of reactive oxygen species (Swords et al. (2010) Blood 115:3796-3800).

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. In one embodiment, the pharmaceutically acceptable salt is a hydrochloride salt form.

As used herein, the term “hypomethylating agent” refers to compounds which inhibit DNA methyltransferase. Examples of hypomethylating agents include azacitidine (also known as azacytidine) and decitabine.

As used herein, a “marker gene” refers to a gene which can have an alteration such that its marker nucleic acid (e.g., DNA or RNA) and/or its marker protein has a characteristic, e.g., size, sequence, composition or amount(s) which, alone or in combination with one or more marker genes, indicate outcome or prognosis upon treatment. Marker genes described herein as linked to outcome after NAE inhibitor, such as pevonedistat or a pharmaceutically acceptable salt thereof treatment are examples of genes within chromosome locus markers and are provided in Table 1 and further described in subsequent paragraphs. Sequences of representative wild type mRNA and proteins corresponding to marker genes also are listed in Table 1. A marker gene listed in Table 1 can have isoforms which are either ubiquitous or have restricted expression. The DNA SEQ ID NOs in Table 1 refer to the mRNA encoding the major or longest wild type isoform and the protein SEQ ID NOs represent at least a precursor of such isoform and not necessarily the mature protein. These sequences are not intended to limit the marker gene identity to that isoform or precursor. The additional wild type isoforms and mature proteins are readily retrievable and understandable to one of skill in the art by reviewing the information provided under the Entrez Gene (database maintained by the National Center for Biotechnology Information, Bethesda, Md.) identified by the Entrez Gene ID number listed in Table 1.

TABLE 1
Marker Gene Description for NAE Inhibitor Treatment

| | | | | | Codons | Example |
|---|---|---|---|---|---|---|
| | | | | | with | of wild |
| | | | | Codons with | Cancer- | type |
| | Entrez | | Chromo- | Cancer-driving | driving | marker |
| Marker | Gene | Ensemble Annotation | some | Single amino acid | Truncating | SEQ ID |
| Gene ID | ID | Transcript | location | changes | Mutations | NOs: |
| TET2 | 54790 | ENST00000540549.1 | 4q24 | 1104-1481; 1843- | All codons | 1, 2 |
| | | | | 2002 | | |
| RUNX1 | 861 | ENST00000344691.4 | 21q22 | 135; 139 | All codons | 3, 4 |
| NRAS | 4893 | ENST00000369535.4 | 1p13 | 12; 13; 14; 18; 24; 50; | none | 5, 6 |
| | | | | 60; 61 | | |
| KRAS | 3845 | ENST00000256078.4 | 12p12 | 12; 13; 14; 17; 19; 22; | none | 7, 8 |
| | | | | 34; 58; 61; 74; 116; | | |
| | | | | 146; 152; 156; 164 | | |
| DNMT3A | 1788 | ENST00000264709.3 | 2p23 | 290-374; 626-910 | All codons | 9, 10 |
| TP53 | 7157 | ENST00000269305.4 | 17p13 | All Codons with | All codons | 11, 12 |
| | | | | ExAc freq < 0.05 | | |
| IDH2 | 3418 | ENST00000330062.3 | 15q26 | 134-146; 164-180 | none | 13, 14 |
| EZH2 | 2146 | ENST00000460911.1 | 7q36 | 1-340; 428- | All codons | 15, 16 |
| | | | | 476; 502-611; 617- | | |
| | | | | 738 | | |
| IDH1 | 3417 | ENST00000415913.1 | 2q34 | 126-138 | none | 17, 18 |
| NPM1 | 4869 | ENST00000296930.5 | 5q35 | None | All codons | 19, 20 |
| PHF6 | 84295 | ENST00000332070.3 | Xq25 | 197-353 | All codons | 21, 22 |
| ASXL1 | 171023 | ENST00000375687.4 | 20q11 | None | 327-1540 | 23, 24 |

In some embodiments, a gene is defined as “genetically altered” or “mutated” “mutant” or as having a“genetic alteration” or a“mutation” if a change from wildtype, e.g., a cancer-driving alteration, e.g., not normal allelic variation, is detected by sequencing a nucleic acid marker corresponding to a marker gene. In one embodiment, the sequencing method is Next Generation Sequencing (NGS). A“truncating mutation” as used herein refers to a sequence change selected from the group consisting of a frameshift insertion, a frameshift deletion, a nonsense mutation, and a splice site mutation in a codon, e.g., a codon described in Table 1, such that the marker nucleic acid encodes, e.g., expression results in, a shortened, typically nonfunctional, altered version of a protein corresponding to the marker gene. A “single amino acid change” as used herein in the context of cancer results from a substituted nucleotide in a codon, e.g., a codon described in Table 1, that encodes, e.g., expression results in a non-reference, cancer driving amino acid in an altered version of a protein corresponding to the marker gene. The reference for the identification of codon number is the Ensemble Annotation Transcript listed in Table 1 and also can be correlated to the protein SEQ ID NOs, e.g., SEQ ID NOs:2, 4, 6, 8, 10, 12, 14, 16, 18, 20, 22 or 24 described in Table 1. In some embodiments, the open reading frame portions of the example nucleic acid marker sequences provided in Table 1 comprise the codons that exhibit the genetic alterations listed in the table. The open reading frames for the sequences are described in the following paragraphs for each marker gene. Ensemble Annotation Transcripts are from the GRCH37 release 94 available at the website maintained by the European Molecular Biology Laboratory-European Bioinformatics Institute (EMBL-EBI).

Genes such as TET2 (reviewed by S. Chiba (2017) Int. J. Hematol. 105:17-22), RUNX1 (reviewed by van Metzeler and Bloomfield (2017) In: Groner et al. (eds), RUNX Proteins in Development and Cancer. Advances in Experimntal Medicine and Biology, 962 962:175-199), NRAS (reviewed with KRAS by Ward et al., Blood 120:3397-3406), KRAS, DNMT3A (reviewed by Yang et al. (2015) Nat. Rev. Cancer 15:152-165), TP53 (studied by Hamadou et al. (2017) Familial Cancer 16:153-157), IDH2 (reviewed with IDH1 by Clark et al. (2016) Clin. Cancer Res. 22:1837-1842), EZH2 (reviewed by Sashida and Iwana (2017) Int. J. Hematol. 105:23-30), IDH1, NPM1 (reviewed by Naoe et al. (2006) Cancer Sci. 97:963-969), PHF6 (studied by Yoo et al. (2012) Acta Oncologica 51:107-111), and ASXL1 (reviewed by Micol and Abdel-Wahab (2016) Cold Spring Harb. Perspect. Med. 6:pii:a026526) are altered in many cancer types.

Frequent gene alterations in CMML involve TET2 (˜60%), ASXL1 (˜40%), SRSF2 (˜50%), RUNX1 (˜15%), SETBP1 (˜10%), RAS (˜30%), and CBL˜15%. (Patnaik and Tefferi (2016) Blood Cancer J. 6:e393. These genes are also frequently altered in AML, which also has alterations in TP53, IDH2 (R172) (Papaemmanuil et al. (2016) N. Engl. J. Med. 374:2209-2221). MDS also shows frequent alterations in TET2, ASXL1, SRSF2, SF3B1, and RUNX1 (Ganguly and Kadam (2016) Mutat. Res. Rev. Mutat. Res. 769:47-62). Further identification of MDS driver alterations, their association with risk and criteria for their identification are described in Lindsley et al. (2017) N. Engl. J. Med. 376:536-547. However, there are other alterations which are more prevalent in AML or MDS than CMML, indicating these diseases have overlapping but not identical genomic landscapes. In a study described herein, patients harboring alterations in TET2, RUNX1, NRAS and KRAS achieved responses (CR+CRi+PR) in 4/4, 3/3, 4/4 and 3/3 cases, respectively. Furthermore, responses were achieved in 4/5 patients harboring alterations in the DNMT3A gene that although low in frequency in CMML (˜5%) is associated with a significant inferior prognosis (Patnaik et al. (2017) Am. J. Hematol. 92:56-61). In some embodiments, genetic alterations typically found in these cancers are listed in Table 1 for the marker genes.

As used herein, “TET2” or “tet methylcytosine dioxygenase 2” refers to Gene ID 54790, the gene corresponding to at least two expressed mRNA variants, one of which is the mRNA described in GenBank Accession No. NM_001127208, SEQ ID NO:1 (open reading frame is nucleotides 488 to 6496 of SEQ ID NO:1), encoding GenPept Accession No. NP_001120680, SEQ ID NO:2). Other names for TET2 include MDS and KIAA1546. TET2 protein functions as a methylcytosine dioxygenase and its gene can be found on chromosome 4 (4q24). TET2 demethylates DNA and is an epigenetic regulator.

As used herein, “RUNX1” or “runt related transcription factor 1” refers to Gene ID 861, the gene corresponding to at least three expressed mRNA variants, one of which is the mRNA described in GenBank Accession No. NM_001001890, SEQ ID NO:3 (open reading frame is nucleotides 1579 to 2860 of SEQ ID NO:3), encoding GenPept Accession No. NP_001001890, SEQ ID NO:4. Other names for RUNX1 include AML1 and core binding factor (CBF) 2 alpha. RUNX1 protein is a transcription factor involved in hematopoiesis and its gene can be found on chromosome 21 (21q22).

As used herein, “NRAS” or “neuroblastoma RAS viral (v-ras) protp-oncogene GTPase” refers to Gene ID 4893, the gene corresponding to the mRNA described in GenBank Accession No. NM_002524, SEQ ID NO:5 (open reading frame is nucleotides 255 to 824 of SEQ ID NO:5), encoding GenPept Accession No. NP_002515, SEQ ID NO:6). Other names for NRAS include Autoimmune Lymphoproliferative Syndrome type IV (ALPS4), NRAS1, and Noonan Syndrome 6 (NS6). NRAS protein functions as an oncogene with GTPase activity and its gene can be found on chromosome 1p (lpl3). NRAS interacts with the cell membrane and various effector proteins, such as Raf and RhoA, which carry out its signaling function through the cytoskeleton and effects on cell adhesion (Fotiadou et al. (2007) Mol. Cel. Biol. 27:6742-6755).

As used herein, “KRAS” or “v-Ki-ras2 Kirsten rat sarcoma viral oncogene homolog” refers to Gene ID 3845, the gene corresponding to at least two expressed mRNA variants, one of which is the mRNA described in GenBank Accession No. NM_004985, SEQ ID NO:7 (open reading frame is nucleotides 193 to 759 of SEQ ID NO:7), encoding GenPept Accession No. NP_004976, SEQ ID NO:8, the predominant transcript variant of KRAS gene on chromosome 12 (12p12). Other names for KRAS include KRAS2, and Noonan Syndrome 3 (NS3). KRAS functions as an oncogene with GTPase activity and can be found on chromosome 12. KRAS interacts with the cell membrane and various effector proteins, such as Akt and Cdc42, which carry out its signaling function through the cytoskeleton and effects on cell motility (Fotiadou et al. supra).

As used herein, “DNMT3A” or “DNA methyltransferase 3 alpha” refers to Gene ID 1788, the gene corresponding to at least six expressed mRNA variants, one of which is the mRNA described in GenBank Accession No. NM_175629, SEQ ID NO:9 (open reading frame is nucleotides 339 to 3077 of SEQ ID NO:9), encoding GenPept Accession No. NP 783328, SEQ ID NO:10. Other names for DNMT3A include TBRS. DNMT3A can be found on chromosome 2 (2p23), methylates DNA and participates in gene silencing and can function during differentiation.

As used herein, “TP53” or “tumor protein p53” refers to Gene ID 7157, the gene corresponding to at least fifteen expressed mRNA variants, one of which is the mRNA described in GenBank Accession No. NM_000546, SEQ ID NO:11 (open reading frame is nucleotides 203 to 1384 of SEQ ID NO:11, or a variant wherein the nucleotide at position 417 is a guanine instead of a cytosine), encoding GenPept Accession No. NP_000537, SEQ ID NO:12 or a variant wherein the amino acid residue at position 72 is an arginine, R instead of a proline, P). Other names for TP53 include BCC7, LFS1 and p53. TP53 can be found on chromosome 17 (17p13), binds DNA and activates transcription factors and can function as a tumor suppressor. Alterations in TP53 have been found to be cancer-driving unless it is seen at a prevalence of ≥0.05 in healthy genomes.

As used herein, “IDH2” or “isocitrate dehydrogenase (NADP(+)) 2” refers to Gene ID 3418, the gene corresponding to at least three expressed mRNA variants, one of which is the mRNA described in GenBank Accession No. NM_002168, SEQ ID NO:13 (open reading frame is nucleotides 165 to 1523 of SEQ ID NO:13), encoding GenPept Accession No. NP_002159, SEQ ID NO:14. Other names for IDH2 include isocitrate dehydrogenase-mitochondrial (ICD-M). IDH2 catalyzes the oxidative decarboxylation of isocitrate to 2-oxoglutarate and can play a role in metabolism. IDH2 can be found on chromosome 15 (15q26) and its protein can permit a tumor to utilize alternative energy pathways.

As used herein, “EZH2” or “enhancer of zeste 2 polycomb repressive complex 2 subunit” refers to Gene ID 2146, the gene corresponding to at least five expressed mRNA variants, one of which is the mRNA described in GenBank Accession No. NM_001203247, SEQ ID NO:15 (open reading frame is nucleotides 194 to 2434 of SEQ ID NO:15), encoding GenPept Accession No. NP_001190176, SEQ ID NO:16. Other names for EZH2 include ENX-1, KMT6, and WVS. EZH2 can be found on chromosome 7 (7q36). EZH2, in combination with other proteins, such as VAV1 oncoprotein, and regulates gene expression through histone methylation and transcriptional silencing.

As used herein, “IDH1” or “isocitrate dehydrogenase (NADP(+)) 1, cytosolic” refers to Gene ID 3417, the gene corresponding to at least three expressed mRNA variants, one of which is the mRNA described in GenBank Accession No. NM_005896, SEQ ID NO:17 (open reading frame is nucleotides 296 to 1540 of SEQ ID NO:17), encoding GenPept Accession No. NP_005887, SEQ ID NO:18. Other names for IDH1 include epididymis luminal protein-216 and oxalosuccinate decarboxylase. IDH1 catalyzes the oxidative decarboxylation of isocitrate to 2-oxoglutarate in the cytoplasm and peroxisome and can play a role in NADPH production. IDH1 can be found on chromosome 2 (2q34) and its protein can permit a tumor to utilize alternative energy pathways.

As used herein, “NPM1” or “nucleophosmin 1” refers to Gene ID 4869, the gene corresponding to at least seven expressed mRNA variants, one of which is the mRNA described in GenBank Accession No. NM_002520, SEQ ID NO:19 (open reading frame is nucleotides 246 to 1130 of SEQ ID NO:19), encoding GenPept Accession No. NP_002511, SEQ ID NO:20. Other names for NPM1 include testicular tissue protein Li 128, nucleolar phosphoprotein B23 and numatrin. NPM1 is a phosphoprotein, is a chaperone of ribosomal proteins and histones from the nucleus to the cytoplasm and can play a role in cell proliferation. NPM1 can be found on chromosome 5 (5q35) and its protein can contribute to tumor growth.

As used herein, “PHF6” or “plant homeodomain-like finger protein 6” refers to Gene ID 84295, the gene corresponding to at least three expressed mRNA variants, one of which is the mRNA described in GenBank Accession No. NM_032458, SEQ ID NO:21 (open reading frame is nucleotides 203 to 1300 of SEQ ID NO:21), encoding GenPept Accession No. NP_115834, SEQ ID NO:22. Other names for PHF6 include PHD-like zinc finger protein and centromere protein 31. PHF6 has zinc finger domains, can localize to the nucleolus and can play a role in transcriptional regulation. PHF6 can be found on chromosome X (Xq26) and its protein can function as a tumor suppressor.

As used herein, “ASXL1” or “additional sex combs like transcriptional regulator 1” refers to Gene ID 171023, the gene corresponding to at least three expressed mRNA variants, one of which is the mRNA described in GenBank Accession No. NM_015338, SEQ ID NO:23 (open reading frame is nucleotides 433 to 5058 of SEQ ID NO:23), encoding GenPept Accession No. NP_056153, SEQ ID NO:24. Other names for ASXL1 include BOPS and MDS. ASXL1 has binds chromatin and can play a role in gene repression. ASXL1 can be found on chromosome 20 (20q11) and its protein can function as a tumor suppressor involved in hematopoietic homeostasis.

There has been interest in public cataloging alterations associated with cancers. Examples of public databases which include information about alterations associated with cancers are the Database of Genotypes and Phenotypes (dbGaP) maintained by the National Center for Biotechnology Information (Bethesda, Md.) and Catalogue of Somatic Mutations in Cancer (COSMIC) database maintained by the Wellcome Trust Sanger Institute (Cambridge, UK). The website maintained by the Exome Aggregation Consortium (ExAC) accessible at the website maintained the Broad Institute, Cambridge, Mass., harmonizes exome sequencing data and provides alternative amino acid frequencies in healthy individuals whose exomes were sequenced as part of various disease-specific and population genetic studies.

The markers and marker genes described herein were identified based on genetic profiles of samples from patients in clinical trials, such as a trial identified on the clinical trials website maintained by the U.S. National Library of Medicine, as NCT01814826. The markers and marker genes were validated using samples from the clinical trial identified as NCT02610777. The patients in these trials were suffering from acute myelogenous leukemia (AML), myelodysplastic syndrome (MDS), or chronic myelomonocytic leukemia (CMML). About 30% of patients suffering from MDS or CMML progress to AML, for which there is no cure. More information about the clinical trial patients, dosing regimens and results can be found in the Examples herein and Swords et al. (2018) Blood 131:1415-1424.

Unless otherwise defined, all technical and scientific terms used herein have the meanings which are commonly understood by one of ordinary skill in the art to which this invention belongs. Generally, nomenclature utilized in connection with, and techniques of cell and tissue culture, molecular biology and protein and oligo- or polynucleotide chemistry and hybridization described herein are those known in the art. GenBank or GenPept accession numbers and useful nucleic acid and peptide sequences can be found at the website maintained by the National Center for Biotechnology Information, Bethesda, Md. The content of all database accession records (e.g., Entrez Gene, GenBank, RefSeq, Ensembl, COSMIC) cited throughout this application (including the Tables) are hereby incorporated by reference. Standard techniques are used for recombinant DNA, oligonucleotide synthesis, protein purification, tissue culture and transformation and transfection (e.g., electroporation, lipofection, etc.). Enzymatic reactions are performed according to manufacturer's specifications or as commonly accomplished in the art or as described herein. The foregoing techniques and procedures generally are performed according to methods known in the art, e.g., as described in various general and more specific references that are cited and discussed throughout the present specification. See e.g., Sambrook et al. (2000)Molecular Cloning: A Laboratory Manual(3^{rd} ed., Cold Spring Harbor Laboratory Press, Cold Spring Harbor, N.Y.) or Harlow, E. and Lane, D. (1988) Antibodies: A Laboratory Manual (Cold Spring Harbor Laboratory Press, Cold Spring Harbor, N.Y.). The nomenclatures utilized in connection with, and the laboratory procedures and techniques of, analytical chemistry, synthetic organic chemistry, and medicinal and pharmaceutical chemistry described herein are known in the art. Standard techniques are used for chemical syntheses, chemical analyses, pharmaceutical preparation, formulation and delivery, and treatment of patients. Furthermore, unless otherwise required by context, singular terms shall include pluralities and plural terms shall include the singular. In the case of conflict, the present specification, including definitions, will control.

The articles “a,” “an” and “at least one” are used herein to refer to one or to more than one of the grammatical object of the article. By way of example, “an element” means one or more than one element, at least one element. In the case of conflict, the present specification, including definitions, will control.

As used herein, the term “about” denotes that the thereafter following value is no exact value but is the center point of a range that is +/−5% of the value of the value. If the value is a relative value given in percentages the term “about” also denotes that the thereafter following value is no exact value but is the center point of a range that is +/−5% of the value, whereby the upper limit of the range cannot exceed a value of 100%.

As used herein, a “favorable” outcome or prognosis refers to long term survival, long progression-free survival (PFS), long time-to-progression (TTP), a negative minimal residual disease (MRD), e.g., at a 10⁵ threshold, and/or good response. Conversely, an “unfavorable” prognosis refers to short term survival, short time-to-progression (TTP), short progression-free survival, a positive minimal residual disease and/or poor response.

A “marker” as used herein, includes a material corresponding to a marker gene whose mutational status has been identified in a biological sample, e.g., tumor cells or contents or products thereof of a patient and furthermore that status is characteristic of a patient whose outcome is favorable or unfavorable with treatment e.g., treatment comprising an NAE inhibitor, such as pevonedistat or a pharmaceutically acceptable salt thereof. Examples of a marker include a material, e.g., marker nucleic acid or marker protein, e.g., a chromosome locus, DNA for a gene, RNA for a gene or protein for or corresponding to a gene. Outcome can be determined using each single marker individually as a marker; or alternatively can include one or more, or all of the characteristics collectively when reference is made to “markers” or “marker sets.” Marker sets can be combinations of chromosome locus, DNA, RNA or protein from more than one marker gene, combinations of chromosome locus, DNA, RNA or protein from a single gene, or any combination of the foregoing. A marker DNA, marker RNA or marker protein can correspond to base pairs on a chromosome locus marker. For example, a marker DNA can include genomic DNA from a chromosome locus marker, marker RNA can include a polynucleotide transcribed from a locus marker, and a marker protein can include a polypeptide resulting from expression at a chromosome locus marker in a biological sample, e.g., =tumor cells or contents or products thereof.

A “marker nucleic acid” is a nucleic acid (e.g., genomic DNA, RNA, cDNA) encoded by or corresponding to a marker gene of the invention. Such marker nucleic acids include DNA, e.g., sense and anti-sense strands of genomic DNA (e.g., including any introns occurring therein), comprising the entire or a partial sequence, e.g., one or more of the exons of the genomic DNA, up to and including the open reading frame of any of the marker genes or the complement of such a sequence. The marker nucleic acids also include RNA comprising the entire or a partial sequence of any marker or the complement of such a sequence, wherein all thymidine residues are replaced with uridine residues, mRNA generated by transcription of genomic DNA (i.e. prior to splicing), and mRNA generated by splicing of RNA transcribed from genomic DNA. A “marker nucleic acid” may also include a cDNA made by reverse transcription of an RNA generated by transcription of genomic DNA (including spliced RNA).

A marker nucleic acid also includes sequences which differ from the wild type nucleotide sequence, e.g., as listed in Table 1, due to degeneracy of the genetic code, and encode wild type protein or protein whose sequence alteration does not affect the healthy state of the subject, e.g., is not a cancer-associated change. It will be appreciated by those skilled in the art that DNA sequence polymorphisms that lead to changes in the amino acid sequence can exist within a population (e.g., the human population). Such genetic polymorphisms can exist among individuals within a population due to natural allelic variation. As used herein, the phrase “allelic variant” refers to a nucleotide sequence which occurs at a given locus or to a polypeptide encoded by the nucleotide sequence. Such naturally occurring allelic variations can typically result in 1-5% variance in the nucleotide sequence of a given gene. Alternative alleles can be identified by sequencing the gene of interest in a number of different individuals, e.g., in cells, e.g., germline cells, of individuals without cancer. Such changes are compiled in the ExAc database and can be readily identified by using hybridization probes to identify the same genetic locus in a variety of individuals. Detection of any and all such nucleotide variations and resulting amino acid polymorphisms or variations that are the result of naturally occurring allelic variation and that do not alter the functional activity of a wild type marker gene is intended to be within the scope of the wild type version of a marker described herein.

A “marker protein” is a protein encoded by a marker nucleic acid or corresponding to a marker, e.g., a mutant nucleic acid, of the invention. For example, a marker protein can be generated by translation of mRNA, e.g., mature or spliced RNA, and includes proteins both before and after cleavage of normally cleaved regions such as transmembrane signal sequences and pro-sequences. The terms “protein” and “polypeptide” are used interchangeably. A protein marker specifically can be referred to by its name or amino acid sequence. It is understood by those skilled in the art, that cancer-associated genetic alterations such as mutations, deletions and/or translocations of marker nucleic acids, e.g., as described for marker genes (e.g., listed in Table 1) can affect protein structure, appearance, cellular location and/or behavior and result in mutant proteins.

As used herein, a “characteristic” of a marker includes a size, sequence, composition or amount whose value or difference is correlated with prognosis or outcome. The characteristic, e.g., size, sequence, composition or amount of a marker can be obtained by analyzing either nucleic acid, e.g., DNA or RNA, or protein corresponding to the marker gene. In some embodiments, a characteristic size of a marker is length or molecular weight. In some embodiments, a characteristic sequence of a marker is a nucleic acid sequence or protein sequence. In some embodiments, a characteristic composition of a marker is nucleotide base or amino acid composition or peptide digest or gene fragment pattern. In some embodiments, a characteristic amount of a marker is copy number and/or expression level. In some embodiments, a characteristic of a marker, e.g., in a sample from a patient, can indicate outcome of treatment if it is different than the characteristic of the wild type or allelic variant of the marker gene. In some embodiments, a characteristic of a marker can indicate outcome if it is wild type. In an embodiment where the amount of a marker is being measured, an amount can indicate outcome if it is greater than or less than a reference amount by a degree greater than the standard error of the assay employed to assess expression. The relative expression level of a marker can be determined upon statistical correlation of the measured expression level and the outcome, e.g., response, time-to-progression, progression-free survival, minimal residual disease or overall survival. The result of the statistical analysis can establish a threshold for selecting markers or marker sets to use in the methods described herein. Alternatively, a marker, e.g., a chromosome locus marker, or a marker gene that has differential characteristic, e.g., size, sequence, composition or amount will have typical ranges that are predictive of outcome, depending on whether the characteristic, e.g., size, sequence, composition or amount falls within the range determined for the outcome. Still further, a set of markers may indicate outcome if the combination of their characteristics, e.g., sizes, sequences, compositions or amounts either meets or is above or below a pre-determined score as determined by methods provided herein. Genetic alterations including, but not limited to, gene translocation, transcript splice variation, deletion and truncation are examples of alterations which can change marker size, sequence or composition, in addition to point mutations which can change marker sequence or composition. Measurement of only one characteristic of a marker gene, e.g., of a marker nucleic acid (i.e., DNA, RNA) or protein can provide a prognosis, i.e., indicate outcome. Measurement of more than one characteristic of a marker gene can provide a prognosis, i.e., indicate outcome when the amounts of the two characteristics are consistent with each other, e.g., the biologies of the results are not contradictory. Examples of consistent results from measurement of multiple characteristics of a marker gene can be identification of a nonsense alteration in a DNA or RNA and a low amount or low molecular weight of encoded protein, or an alteration in a region which encodes a binding pocket or active site of a protein and low activity of the encoded protein. A different example can occur when a protein is in a pathway with a feedback loop controlling its synthesis based on its activity level. In this example, a low amount or activity of protein can be associated with a high amount of its altered mRNA as a tissue, due to the marker gene alteration, thus is starved for the protein activity and repeatedly signals the production of the protein.

As used herein, “gene deletion” refers to an amount of DNA copy number less than 2 and “amplification” refers to an amount of DNA copy number greater than 2. A “diploid” amount refers to a copy number equal to 2. The term “diploid or amplification” can be interpreted as “not deletion” of a gene copy. Conversely, the term “diploid or deletion” can be interpreted as “not amplification” of copy number. For the sake of clarity, sequence deletion can occur within a gene as a result of marker gene alteration and can result in absence of transcribed protein or a shortened mRNA or protein. Such a deletion may not affect copy number.

The terms “long term survival,” “long overall survival,” “short term survival” and “short overall survival” refer to the length of time after receiving a first dose of treatment that a cancer patient is predicted to live. A “long term survivor” refers to a patient expected have a slower rate of progression or later death from the tumor than those patients identified as short term survivors. “Enhanced survival” or “a slower rate of death” are estimated life span determinations based upon characteristic, e.g., size, sequence, composition or amount of one or more of marker genes described herein, e.g., as compared to a reference standard such that 70%, 80%, 90% or more of the population will be alive a sufficient time period after receiving a first dose of treatment. A “faster rate of death” or “shorter survival time” refer to estimated life span determinations based upon characteristic, e.g., size, sequence, composition or amount of one or more of marker genes described herein, e.g., as compared to a reference standard such that 50%, 40%, 30%, 20%, 10% or fewer of the population will not live a sufficient time period after receiving a first dose of treatment. In some embodiments, the sufficient time period is at least 12, 18, 24 or 30 months or 3 years, 4 years or 5 years as measured from the first day of receiving a cancer therapy.

In some embodiments, a cancer is “responsive” to a therapeutic agent or there is a “good response” to a therapeutic regimen if its rate of growth is inhibited as a result of contact with a therapeutic agent, compared to its growth in the absence of contact with the therapeutic agent or one or more symptoms of the cancer are ameliorated. Growth of a cancer can be measured in a variety of ways, for example, the size of a tumor or the expression of tumor markers appropriate for that tumor type may be measured. International Working Groups convene periodically to set, update and publish disease and response criteria for various types of cancers. Such published reports can be followed to support the identification of markers of the subject tumors and their response to NAE inhibitors. Examples are criteria for Acute Myelogenous Leukemia (AML, Cheson et al. (2003) J. Clin. Oncol. 21:4642-4649), chronic myelomonocytic leukemia (CML, Swerdlow et al. eds. (2008) in WHO Classification of Tumours of Haemotopoietic and Lymphoid Tissues (4^{th} edition, IARC Press), lymphomas, e.g., non-Hodgkin's and Hodgkin's lymphoma (Cheson et al. (2007) J. Clin. Oncol. 25:579-596). Examples used to support the identification of myeloma and its response to a therapeutic regimen include the Southwestern Oncology Group (SWOG) criteria as described in Blade et al. (1998) Br J Haematol. 102:1115-23 can be used. These criteria define the type of response measured in myeloma and also the characterization of time to disease progression which is another important measure of a tumor's sensitivity to a therapeutic agent. Criteria take into account analysis methods such as Positron Emission Tomography (PET), e.g., for identifying sites with measurable altered metabolic activity (e.g., at tumor sites) or to trace specific markers into tumors in vivo, immunohistochemistry, e.g., to identify tumor cells by detecting binding of antibodies to specific tumor markers, and flow cytometry, e.g., to characterize cell types by differential markers and fluorescent stains, in addition to traditional methods such as histology to identify cell composition (e.g., blast counts in a blood smear or a bone marrow biopsy, presence and number of mitotic figures) or tissue structure (e.g., disordered tissue architecture or cell infiltration of basement membrane). In some embodiments, the quality of being responsive to a therapy comprising an NAE inhibitor, such as pevonedistat or a pharmaceutically acceptable salt thereof can be a variable one, with different cancers exhibiting different levels of “responsiveness” to a given therapeutic agent, under different conditions. Still further, measures of responsiveness can be assessed using additional criteria beyond growth size of a tumor, including, but not limited to, patient quality of life, degree of metastases. In addition, clinical prognostic markers and variables can be assessed (e.g., M protein in myeloma, PSA levels in prostate cancer) in applicable situations.

In some embodiments, a cancer is “non-responsive” or has a “poor response” to a therapeutic agent or therapeutic regimen if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or the expression of tumor markers appropriate for that tumor type may be measured. For example, the response definitions used to support the identification of markers associated with non-response of tumors to therapeutic agents, guidelines such as those described above can be used. In some embodiments, the quality of being non-responsive to a therapeutic agent can be a highly variable one, with different cancers exhibiting different levels of “non-responsiveness” to a given therapeutic agent, under different conditions. Still further, measures of non-responsiveness can be assessed using additional criteria beyond growth size of a tumor, including, but not limited to, patient quality of life, degree of metastases. In addition, clinical prognostic markers and variables can be assessed (e.g., M protein in myeloma, PSA levels in prostate cancer) in applicable situations.

As used herein, “long time-to-progression, “long TTP” and “short time-to-progression,” “short TTP” refer to the amount of time until when the stable disease brought by treatment converts into an active disease. On occasion, a treatment results in stable disease which is neither a good nor a poor response, e.g., MR, the disease merely does not get worse, e.g., become a progressive disease, for a period of time. This period of time can be at least 4-8 weeks, at least 3-6 months or more than 6 months.

As used herein, “progression free survival” or “PFS” refers to the time elapsed between treatment initiation and tumor progression or death from any cause.

As used herein, “minimal residual disease” or “MRD” refers to the result of an assay to detect residual malignant cancer or tumor cells in a patient, e.g., after at least some treatment with a therapeutic regimen. MRD negative refers to a result when no residual tumor cells can be found in a sample from the patient. MRD positive refers to a result when a small number of tumor cells can be found in a sample from the patient. MRD can be qualified by an assay threshold, e.g., a 10⁻⁴, a 10⁻⁵ or a 10⁻⁶ threshold, i.e., related to a limit of detection of the assay, such as flow cytometry for detecting tumor cells.

“Treatment” as used herein in the context of cancer shall mean the use of a therapy to prevent or inhibit further tumor growth, to cause shrinkage of a tumor, alleviate tumor burden, and/or to provide longer survival times. Treatment is also intended to include prevention of metastasis of tumor. A tumor is “inhibited” or “treated” (e.g., as determined by responsiveness, time to progression, progression-free survival, minimal residual disease or indicators known in the art and described herein) if at least one symptom of the cancer or tumor is alleviated, terminated, slowed, minimized, or prevented. Any amelioration of any symptom, physical or otherwise, of a tumor pursuant to treatment using a therapeutic regimen (e.g., comprising an NAE inhibitor, such as comprising pevonedistat or a pharmaceutically acceptable salt thereof) as further described herein, is within the scope of the invention.

As used herein, the term “agent” is defined broadly as anything that cancer cells, including tumor cells, may be exposed to in a therapeutic regimen. In the context of the present invention, such agents include, but are not limited to, an NAE inhibitor, such as pevonedistat or a pharmaceutically acceptable salt thereof, as well as chemotherapeutic agents used in combination with the NAE inhibitor, as known in the art and described in further detail herein.

The term “probe” refers to any molecule, e.g., an isolated molecule, which is capable of selectively binding to a specifically intended target molecule, for example a marker of the invention. Probes can be either synthesized by one skilled in the art or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

A “normal” or “reference” characteristic, e.g., size, sequence, composition or amount of a marker may refer to the characteristic, e.g., size, sequence, composition or amount in a “reference sample.” A reference sample can be a matched normal or control, e.g., germline, sample from the same patient from whom the cancer sample is derived. A reference sample can be a sample from a healthy subject not having the marker-associated disease or having a reference characteristic e.g., the average characteristic, e.g., size, sequence, composition or amount of the wild type marker in several healthy subjects. A reference sample characteristic, e.g., size, sequence, composition or amount may be comprised of a characteristic, e.g., size, sequence, composition or amount of one or more markers from a reference database. Alternatively, a “normal” characteristic, e.g., size, sequence, composition or amount of a marker is the characteristic, e.g., size, sequence, composition or amount of the marker, e.g., marker gene in non-tumor cells in a similar environment or response situation from the same patient from whom the tumor is derived. The normal amount of DNA copy number is 2 or diploid, with the exception of X-linked genes in males, where the normal DNA copy number is 1.

“Over-expression” or “upregulation” and “under-expression” or “downregulation” of a marker gene, refer to expression of the marker gene of a patient at a greater or lesser level (e.g. more than three-halves-fold, at least two-fold, at least three-fold, greater or lesser level etc.), respectively, than normal level of expression of the marker gene, e.g., as measured by mRNA or protein, in a test sample that is greater than the standard error of the assay employed to assess expression. A “significant” expression level may refer to a level which either meets or is above or below a pre-determined score for a marker gene set as determined by methods provided herein.

“Complementary” in the context of nucleic acids, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In an embodiment, the first region comprises a first portion and the second region comprises a second portion, whereby, when the first and second portions are arranged in an antiparallel fashion, at least about 50%, at least about 75%, at least about 90%, or at least about 95% or all of the nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue (i.e., by percent identity). By way of example, a region having the nucleotide sequence 5′-ATTGCC-3′ and a region having the nucleotide sequence 5′-TATGGC-3′ share homology with 50% identity. In one embodiment, the first region comprises a first portion and the second region comprises a second portion, whereby, at least about 50%, at least about 75%, at least about 90%, or at least about 95% of the nucleotide residue positions of each of the portions are occupied by the same nucleotide residue. In an embodiment of 100% identity, all nucleotide residue positions of each of the portions are occupied by the same nucleotide residue.

Unless otherwise specified herein, the terms “antibody” and “antibodies” broadly encompass naturally-occurring forms of antibodies, e.g., polyclonal antibodies (e.g., IgG, IgA, IgM, IgE) and monoclonal and recombinant antibodies such as IgG, single-chain antibodies, two-chain and multi-chain proteins, chimeric, CDR-grafted, human and humanized antibodies and multi-specific antibodies, as well as fragments and derivatives of all of the foregoing, which fragments (e.g., dAbs, scFv, Fab, F(ab)′₂, Fab′) and derivatives having at least an antigenic binding site. Antibody derivatives may comprise a protein or chemical moiety conjugated to an antibody. The term “antibody” also includes synthetic and genetically engineered variants.

A “kit” is any article of manufacture (e.g., a package or container) comprising at least one reagent, e.g. a probe, for specifically detecting a marker or marker set of the invention. The article of manufacture may be promoted, distributed, sold or offered for sale as a unit for performing, e.g., in vitro, the methods of the present invention, e.g., on a sample having been obtained from a patient, e.g., a human patient. The reagents included in such a kit can comprise at least one probe, such as a nucleic acid probe and, optionally, one or more primers and/or at least one antibody probe, for use in detecting marker characteristics, e.g., size, sequence composition or amount, e.g., expression. In addition, a kit of the present invention can contain instructions which describe a suitable detection assay. Such a kit can be conveniently used, e.g., in a clinical or a contract testing setting, to generate results, e.g., on characteristic, e.g., size, sequence, composition or amount of one or more marker, to be recorded, stored, transmitted or received to allow for diagnosis, evaluation or treatment of patients exhibiting symptoms of cancer, in particular patients exhibiting the possible presence of a cancer capable of treatment with a regimen comprising NAE inhibition therapy, including, e.g., hematological cancers e.g., myelomas (e.g., multiple myeloma), lymphomas (e.g., non-Hodgkin's lymphoma), leukemias (e.g., acute myelogenous leukemia, chronic myelomonocytic leukemia, myelodysplastic syndrome), or solid tumors (e.g., tumors of skin, lung, breast, ovary).

The present methods and compositions are designed for use in diagnostics and therapeutics for a patient suffering from cancer. A cancer or tumor is treated or diagnosed according to the present methods. “Cancer” or “tumor” is intended to include any neoplastic growth in a patient, including an initial tumor and any metastases. The cancer can be of the hematological or solid tumor type. Hematological tumors include tumors of hematological origin, including, e.g., myelomas (e.g., monoclonal gammopathy of undetermined significance (MGUS), plasmacytoma, smoldering myeloma, multiple myeloma), leukemias (e.g., Waldenstrom's syndrome, chronic lymphocytic leukemia, acute myelogenous leukemia, chronic myelogenous leukemia, chronic myelomonocytic leukemia, other leukemias), lymphomas (e.g., B-cell lymphomas, e.g., diffuse large B-cell lymphoma, non-Hodgkin's lymphoma) and myelodysplastic syndrome. In some embodiments, the MDS is high risk MDS, typically characterized by more than 5% of the bone marrow comprised of immature blast cells. In some embodiments, the AML is low-blast acute AML. Solid tumors can originate in organs or can metastasize from other tumors, and include cancers such as, but not limited to, in skin, lung, brain, breast, prostate, ovary, colon, kidney, pancreas, liver, esophagus, stomach, intestine, bladder, uterus, cervix, head and neck, central nervous system, bone, testis, and adrenal gland. The cancer can comprise a cell in which a marker gene has an alteration. As used herein, cancer cells, including tumor cells, refer to cells that divide at an abnormal (increased) rate or whose control of growth or survival is different than for cells in the same tissue where the cancer cell arises or lives. Cancer cells include, but are not limited to, cells in carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; cells in hematologic cancers, such as myelomas, leukemias (e.g., acute myelogenous leukemia, chronic lymphocytic leukemia, chronic myelomonocytic leukemia, granulocytic leukemia, monocytic leukemia, lymphocytic leukemia), and cells in lymphomas (e.g., follicular lymphoma, mantle cell lymphoma, diffuse large B-cell lymphoma, malignant lymphoma, plasmocytoma, reticulum cell sarcoma, or Hodgkins disease); and cells in tumors of the nervous system including glioma, meningoma, medulloblastoma, schwannoma or epidymoma.

As used herein, the term “noninvasive” refers to a procedure which inflicts minimal harm to a subject. In the case of clinical applications, a noninvasive sampling procedure can be performed quickly, e.g., in a walk-in setting, typically without anaesthesia and/or without surgical implements or suturing. Examples of noninvasive samples include, but are not limited to, blood, serum, saliva, urine, buccal swabs, throat cultures, stool samples and cervical smears. Noninvasive diagnostic analyses include x-rays, magnetic resonance imaging, positron emission tomography.

Described herein is the assessment of outcome for treatment of a tumor through measurement of one or more characteristics of a marker gene. Also described are assessing the outcome by noninvasive, convenient or low-cost means, for example, from blood samples. Typical methods to determine extent of cancer or outcome of a cancer, e.g., a hematological cancer, e.g., lymphoma, leukemia, e.g., AML, CMML, MDS, myeloma (e.g., multiple myeloma) can employ bone marrow biopsy to collect tissue for genotype or phenotype, e.g., histological analysis. The invention provides methods for determining, assessing, advising or providing an appropriate therapy regimen for treating a tumor or managing cancer in a patient. Monitoring a treatment using the kits and methods disclosed herein can identify the potential for unfavorable outcome and allow their prevention, and thus a savings in morbidity, mortality and treatment costs through adjustment in the therapeutic regimen, cessation of therapy or use of alternative therapy.

The term “biological sample” is intended to include a material, e.g., tissue, cells, biological fluids and isolates thereof, obtained, e.g., isolated or collected, from a subject, e.g., a human, such as a patient or a normal subject. A tumor sample from a cancer patient can comprise tumor cells or contents or products thereof. In hematological cancers of the bone marrow, e.g., leukemias or myeloma, a sample for primary analysis of the tumor can be a bone marrow sample. However, some tumor cells, (e.g., clonotypic tumor cells, circulating endothelial cells), are a percentage of the cell population in whole blood. Bone marrow cells also can be mobilized into the blood during treatment of the patient with granulocyte-colony stimulating factor (G-CSF) in preparation for a bone marrow transplant, a standard treatment for hematological cancers, e.g., leukemias, lymphomas and myelomas. Examples of circulating tumor cells in multiple myeloma have been studied e.g., by Pilarski et al. (2000) Blood 95:1056-65 and Rigolin et al. (2006) Blood 107:2531-5. Thus, noninvasive samples, e.g., for in vitro measurement of markers to determine outcome of treatment, can include peripheral blood samples. Accordingly, cells within peripheral blood can be tested for marker characteristics. For patients with cancer, e.g., hematological cancer, a control, reference sample for normal characteristic, e.g., size, sequence, composition or amount can be obtained from skin or a buccal swab of the patient. For solid tumors, a typical tumor sample is a biopsy of the tumor and thus comprises solid tumor cells. Alternatively, a sample of tumor cells shed or scraped from the tumor site can be collected noninvasively, such as, but not limited to, in blood, sputum, a nipple aspirate, urine, stool, cervical smear. For solid tumors, a control reference sample for normal characteristic, e.g., size, sequence, composition or amount can be obtained from blood of the patient.

Blood collection containers can comprise an anti-coagulant, e.g., heparin or ethylene-diaminetetraacetic acid (EDTA), sodium citrate or citrate solutions with additives to preserve blood integrity, such as dextrose or albumin or buffers, e.g., phosphate. If the amount of marker is being measured by measuring the level of its DNA in the sample, a DNA stabilizer, e.g., an agent that inhibits DNAse, can be added to the sample. If the amount of marker is being measured by measuring the level of its RNA in the sample, an RNA stabilizer, e.g., an agent that inhibits RNAse, can be added to the sample. If the amount of marker is being measured by measuring the level of its protein in the sample, a protein stabilizer, e.g., an agent that inhibits proteases, can be added to the sample. An example of a blood collection container is PAXGENE® tubes (PREANALYTIX, Valencia, Calif.), useful for RNA stabilization upon blood collection. Peripheral blood samples can be modified, e.g., fractionated, sorted or concentrated (e.g., to result in samples enriched with tumor or depleted of tumor (e.g., for a reference sample)). Examples of modified samples include clonotypic myeloma cells, which can be collected by e.g., negative selection, e.g., separation of white blood cells from red blood cells (e.g., differential centrifugation through a dense sugar or polymer solution (e.g., FICOLL® solution (Amersham Biosciences division of GE healthcare, Piscataway, N.J.) or HISTOPAQUE®-1077 solution, Sigma-Aldrich Biotechnology LP and Sigma-Aldrich Co., St. Louis, Mo.)) and/or positive selection by binding B cells to a selection agent (e.g., a reagent which binds to a tumor cell or myeloid progenitor marker, such as CD34, CD38, CD138, or CD133, for direct isolation (e.g., the application of a magnetic field to solutions of cells comprising magnetic beads (e.g., from Miltenyi Biotec, Auburn, Calif.) which bind to the B cell markers) or fluorescent-activated cell sorting).

Alternatively, a tumor cell line, e.g., OCI-Ly3, OCI-Ly10 cell (Alizadeh et al. (2000) Nature 403:503-511), a RPMI 6666 cell, a SUP-B15 cell, a KG-1 cell, a CCRF-SB cell, an 8ES cell, a Kasumi-1 cell, a Kasumi-3 cell, a BDCM cell, an HL-60 cell, a Mo-B cell, a JM1 cell, a GA-10 cell or a B-cell lymphoma (e.g., BC-3) or a cell line or a collection of tumor cell lines (see e.g., McDermott et al. (2007) PNAS 104:19936-19941 or ONCOPANEL™ anti-cancer tumor cell profiling screen (Ricerca Biosciences, Bothell, Wash.)) can be assayed. A skilled artisan readily can select and obtain the appropriate cells (e.g., from American Type Culture Collection (ATCC®), Manassas, Va.) that are used in the present method. If the compositions or methods are being used to predict outcome of treatment in a patient or monitor the effectiveness of a therapeutic protocol, then a tissue or blood sample having been obtained from the patient being treated is a useful source of cells or marker gene or gene products for an assay.

The biological sample, e.g., tumor, e.g., biopsy or bone marrow, blood or modified blood, (e.g., comprising tumor cells) and/or the reference, e.g., matched control (e.g., germline), sample can be subjected to a variety of well-known post-collection preparative and storage techniques (e.g., nucleic acid and/or protein extraction, fixation, storage, freezing, ultrafiltration, concentration, evaporation, centrifugation) prior to assessing the amount of the marker in the sample.

### Assay Methods

In an embodiment, mutational status of a marker gene, e.g., whether wild type or comprising a gene alteration in a marker can be identified by sequencing a nucleic acid, e.g., a DNA, RNA, cDNA or a protein correlated with the marker gene. There are several sequencing methods known in the art to sequence nucleic acids. A nucleic acid primer can be designed to bind to a region comprising a potential alteration site or can be designed to complement the altered sequence rather than the wild type sequence. Primer pairs can be designed to bracket a region comprising a potential alteration in a marker gene. A primer or primer pair can be used for sequencing one or both strands of DNA corresponding to the marker gene. A primer can be used in conjunction with a probe, e.g., a nucleic acid probe, e.g., a hybridization probe, to amplify a region of interest prior to sequencing to boost sequence amounts for detection of an alteration in a marker gene. Examples of regions which can be sequenced include an entire gene, transcripts of the gene and a fragment of the gene or the transcript, e.g., one or more of exons or untranslated regions. Examples of alterations to target for primer selection and sequence or composition analysis can be found in public databases which collect alteration information, such as COSMIC and dbGaP. Some altered portions of marker genes such as TET2, RUNX1, NRAS, KRAS, DNMT3A, TP53, IDH2, EZH2, IDH1, NPM1, PHF6, and/or ASXL1 are listed in Table 1 as examples of alterations that can be associated with sensitivity to NAE inhibition, e.g., pevonedistat or a pharmaceutically acceptable salt thereof.

Sequencing methods are known to one skilled in the art. Examples of methods include the Sanger method, the SEQUENOM™ method and Next Generation Sequencing (NGS) methods. The Sanger method, comprising the use of electrophoresis, e.g., capillary electrophoresis to separate primer-elongated labeled DNA fragments, can be automated for high-throughput applications. The primer extension sequencing can be performed after PCR amplification of regions of interest. Software can assist with sequence base calling and with alteration identification. SEQUENOM™ MASSARRAY® sequencing analysis (San Diego, Calif.) is a mass-spectrometry method which compares actual mass to expected mass of particular fragments of interest to identify alterations. NGS technology (also called “massively parallel sequencing” and “second generation sequencing”) in general provides for much higher throughput than previous methods and uses a variety of approaches (reviewed in Zhang et al. (2011) J. Genet. Genomics 38:95-109 and Shendure and Hanlee (2008) Nature Biotech. 26:1135-1145). NGS methods can identify low frequency alterations in a marker in a sample. Some NGS methods (see, e.g., GS-FLX Genome Sequencer (Roche Applied Science, Branford, Conn.), Genome analyzer (Illumina, Inc. San Diego, Calif.), SOLID™ analyzer (Applied Biosystems, Carlsbad, Calif.), Polonator G.007 (Dover Systems, Salem, N.H.), HELISCOPE™ (Helicos Biosciences Corp., Cambridge, Mass.)) use cyclic array sequencing, with or without clonal amplification of PCR products spatially separated in a flow cell and various schemes to detect the labeled modified nucleotide that is incorporated by the sequencing enzyme (e.g., polymerase or ligase). In one NGS method, primer pairs can be used in PCR reactions to amplify regions of interest. Amplified regions can be ligated into a concatenated product. Clonal libraries are generated in the flow cell from the PCR or ligated products and further amplified (“bridge” or “cluster” PCR) for single-end sequencing as the polymerase adds a labeled, reversibly terminated base that is imaged in one of four channels, depending on the identity of the labeled base and then removed for the next cycle. Software can aid in the comparison to genomic sequences to identify alterations.

Composition of proteins and nucleic acids can be determined by many ways known in the art, such as by treating them in ways that cleave, degrade or digest them and then analyzing the components. Mass spectrometry, electrophoresis and chromatography can separate and define components for comparison. Alterations which cause deletions or insertions can be identified by size or charge differences in these methods. Protein digestion or restriction enzyme nucleic acid digestion can reveal different fragment patterns after some alterations. Antibodies that recognize particular mutant amino acids in their structural contexts can identify and detect these alterations in samples (see below).

In an embodiment, DNA, e.g., genomic DNA corresponding to the wild type or altered marker gene, can be analyzed both by in situ and by in vitro formats in a biological sample using methods known in the art. DNA can be directly isolated from the sample or isolated after isolating another cellular component, e.g., RNA or protein. Kits are available for DNA isolation, e.g., QIAAMP® DNA Micro Kit (Qiagen, Valencia, Calif.). DNA also can be amplified using such kits.

In another embodiment, mRNA corresponding to the marker gene can be analyzed both by in situ and by in vitro formats in a biological sample using methods known in the art. An example of a method for measuring expression level is included in the Examples. For example, a nucleic acid probe can be used to hybridize to a marker and the amount of probe hybridized can be measured. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). RNA can be isolated using standard procedures (see e.g., Chomczynski and Sacchi (1987) Anal. Biochem. 162:156-159), solutions (e.g., trizol, TRI REAGENT® (Molecular Research Center, Inc., Cincinnati, Ohio; see U.S. Pat. No. 5,346,994) or kits (e.g., a QIAGEN® Group RNEASY® isolation kit (Valencia, Calif.) or LEUKOLOCK™ Total RNA Isolation System, Ambion division of Applied Biosystems, Austin, Tex.).

Additional steps may be employed to remove DNA from RNA samples. Cell lysis can be accomplished with a nonionic detergent, followed by microcentrifugation to remove the nuclei and hence the bulk of the cellular DNA. DNA subsequently can be isolated from the nuclei for DNA analysis. In one embodiment, RNA is extracted from cells of the various types of interest using guanidinium thiocyanate lysis followed by CsCl centrifugation to separate the RNA from DNA (Chirgwin et al. (1979) Biochemistry 18:5294-99). Alternatively, separation of RNA from DNA can be accomplished by organic extraction, for example, with hot phenol or phenol/chloroform/isoamyl alcohol. If desired, RNAse inhibitors may be added to the lysis buffer. Likewise, for certain cell types, it may be desirable to add a protein denaturation/digestion step to the protocol. For many applications, it is desirable to enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX.R™ medium (see Ausubel et al. (1994) Current Protocols In Molecular Biology, vol. 2, Current Protocols Publishing, New York). Once bound, poly(A)+mRNA is eluted from the affinity column using 2 mM EDTA/0.1% SDS. (see Sambrook et al. (1989) Molecular Cloning—A Laboratory Manual(2nd ed.), Cold Spring Harbor Laboratory, Cold Spring Harbor, N.Y.).

The characteristic of a marker of the invention in a biological sample, e.g., after obtaining a biological sample (e.g., a bone marrow sample, a tumor biopsy, a sample comprising tumor cells, tumor cell products or tumor cell components or a reference sample) from a subject, may be detected or measured by any of a wide variety of well-known methods with a nucleic acid (e.g., RNA, mRNA, genomic DNA, or cDNA) and/or translated protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. These methods include gene array/chip technology, RT-PCR, TAQMAN® gene expression assays (Applied Biosystems, Foster City, Calif.), e.g., under GLP approved laboratory conditions, in situ hybridization, immunohistochemistry, immunoblotting, FISH (fluorescence in situ hybridization), FACS analyses, northern blot, southern blot, INFINIUM® DNA analysis Bead Chips (Illumina, Inc., San Diego, Calif.), quantitative PCR, bacterial artificial chromosome arrays, single nucleotide polymorphism (SNP) arrays (Affymetrix, Santa Clara, Calif.) or cytogenetic analyses. The detection methods of the invention can thus be used to detect mutational status in RNA, mRNA, protein, cDNA, or genomic DNA, for example, in a biological sample in vitro as well as in vivo. Furthermore, in vivo techniques for detection of a polypeptide or nucleic acid corresponding to a marker of the invention include introducing into a subject a labeled probe to detect the biomarker, e.g., a nucleic acid complementary to the transcript of a biomarker or a labeled antibody, Fc receptor or antigen directed against the polypeptide, e.g., wild type or mutant marker. For example, the antibody can be labeled with a radioactive isotope whose presence and location in a subject can be detected by standard imaging techniques. These assays can be conducted in a variety of ways. A skilled artisan can select from these or other appropriate and available methods based on the nature of the marker(s), tissue sample and alteration in question. Different methods or combinations of methods could be appropriate in different cases or, for instance in different types of tumors or patient populations.

In some embodiments, detection assays involve preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways. For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay. One example of such an embodiment includes use of an array or chip which contains a marker or marker set anchored for expression analysis of the sample.

There are many established methods for anchoring assay components to a solid phase, e.g., glass, polystyrene, nylon, polypropylene, nylon, polyethylene, dextran, amylases, natural and modified celluloses, polyacrylamides, gabbros, and magnetite. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

In order to conduct assays with the above-mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In an embodiment, the probe, when it is the unanchored assay component, can be labeled for the purpose of detection and readout of the assay, either directly or indirectly, with detectable labels discussed herein and which are well-known to one skilled in the art. The term “labeled”, with regard to the probe (e.g., nucleic acid or antibody), is intended to encompass direct labeling of the probe by coupling (i.e., physically linking) a detectable substance to the probe, as well as indirect labeling of the probe by reactivity with another reagent that is directly labeled. The label can be a radioisotope, a fluorescent compound, an enzyme, an enzyme co-factor, a hapten, a sequence tag, a protein or an antibody. An example of indirect labeling includes detection of a primary antibody using a fluorescently labeled secondary antibody. It is also possible to directly detect marker/probe complex formation without further manipulation or labeling of either component (marker or probe), for example by utilizing the technique of fluorescence energy transfer (FET, see, for example, Lakowicz et al., U.S. Pat. No. 5,631,169; Stavrianopoulos, et al., U.S. Pat. No. 4,868,103).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). As used herein, “BIA” or “surface plasmon resonance” is a technology for studying biospecific interactions in real time, without labeling any of the interactants (e.g., BIACORE™). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous detection assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. Appropriate conditions to the particular assay and components thereof will be well known to one skilled in the art.

Nucleic acid probes of the invention may be prepared by chemical synthesis using any suitable methodology known in the art, may be produced by recombinant technology, or may be derived from a biological sample, for example, by restriction digestion. The nucleic acids can be modified at the base moiety, at the sugar moiety, or at the phosphate backbone. An example of a nucleic acid label is incorporated using SUPER™ Modified Base Technology (Nanogen, Bothell, Wash., see U.S. Pat. No. 7,045,610). The level of expression can be measured as general nucleic acid levels, e.g., after measuring the amplified DNA levels (e.g. using a DNA intercalating dye, e.g., the SYBR green dye (Qiagen Inc., Valencia, Calif.) or as specific nucleic acids, e.g., using a probe-based design, with the probes labeled. TAQMAN® assay formats can use the probe-based design to increase specificity and signal-to-noise ratio.

Hybridization of an RNA or a cDNA with the nucleic acid probe can indicate that the marker in question is being expressed. As used herein, the term “hybridizes” is intended to describe conditions for hybridization and washing under which nucleotide sequences that are significantly identical or homologous to each other remain hybridized to each other. In some embodiments, the conditions are such that sequences at least about 70%, at least about 80%, at least about 85%, 90% or 95% identical to each other remain hybridized to each other for subsequent amplification and/or detection. Stringent conditions vary according to the length of the involved nucleotide sequence but are known to those skilled in the art and can be found or determined based on teachings in Current Protocols in Molecular Biology, Ausubel et al., eds., John Wiley & Sons, Inc. (1995), sections 2, 4 and 6. Additional stringent conditions and formulas for determining such conditions can be found in Molecular Cloning: A Laboratory Manual, Sambrook et al., Cold Spring Harbor Press, Cold Spring Harbor, N.Y. (1989), chapters 7, 9 and 11. A non-limiting example of stringent hybridization conditions for hybrids that are at least 10 basepairs in length includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A non-limiting example of highly stringent hybridization conditions for such hybrids includes hybridization in 1×SSC, at about 65-70° C. (or hybridization in 1×SSC plus 50% formamide at about 42-50° C.) followed by one or more washes in 0.3×SSC, at about 65-70° C. A non-limiting example of reduced stringency hybridization conditions for such hybrids includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6 (log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, polyvinylpyrrolidone (PVP) and the like. Nucleic acid probes of the invention can refer to nucleic acids which hybridize to the region of interest and which are not further extended. For example, it specifically hybridizes to a mutant region of a biomarker, and which by hybridization or absence of hybridization to the DNA of a patient or the type of hybrid formed can be indicative of the presence or identity of the alteration of the biomarker or the amount of marker activity.

In one format, the RNA is immobilized on a solid surface and contacted with a probe, for example by running the isolated RNA on an agarose gel and transferring the RNA from the gel to a membrane, such as nitrocellulose. In an alternative format, the nucleic acid probe(s) are immobilized on a solid surface and the RNA is contacted with the probe(s), for example, in an AFFYMETRIX® gene chip array or a SNP chip (Santa Clara, Calif.) or customized array using a marker set comprising at least one marker indicative of treatment outcome. A skilled artisan can readily adapt known RNA and DNA detection methods for use in detecting the amount of the markers of the present invention. For example, the high density microarray or branched DNA assay can benefit from a higher concentration of tumor cell in the sample, such as a sample which had been modified to isolate tumor cells as described in earlier sections. In a related embodiment, a mixture of transcribed polynucleotides obtained from the sample is contacted with a substrate having fixed thereto a polynucleotide complementary to or homologous with at least a portion (e.g., at least 7, 10, 15, 20, 25, 30, 40, 50, 100, 500, or more nucleotide residues) of a marker nucleic acid. If polynucleotides complementary to or homologous with the marker are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). In an embodiment when a method of assessing marker expression is used which involves hybridization of one nucleic acid with another, the hybridization can be performed under stringent hybridization conditions.

For in situ methods, RNA does not need to be isolated from the cells prior to detection. In such methods, a cell or tissue sample is prepared/processed using known histological methods. The sample is then immobilized on a support, typically a glass slide, and then contacted with a probe that can hybridize to RNA that encodes the marker.

In vitro techniques for detection of a polypeptide corresponding to a marker of the invention include enzyme linked immunosorbent assays (ELISAs), Western blots, protein array, immunoprecipitations and immunofluorescence. In such examples, expression of a marker is assessed using an antibody (e.g., a radio-labeled, chromophore-labeled, fluorophore-labeled, or enzyme-labeled antibody), an antibody derivative (e.g., an antibody conjugated with a substrate or with the protein or ligand of a protein-ligand pair (e.g., biotin-streptavidin)), or an antibody fragment (e.g., a single-chain antibody, an isolated antibody hypervariable domain) which binds specifically with a marker protein or fragment thereof, e.g., a protein or fragment comprising a region which can be altered or a portion comprising an altered sequence, or an altered residue in its structural context, including a marker protein which has undergone all or a portion of its normal post-translational modification. An antibody can detect a marker gene protein described herein, e.g., a protein corresponding to TET2, RUNX1, NRAS, KRAS, DNMT3A, TP53, IDH2, EZH2, IDH1, NPM1, PHF6, or ASXL1, e.g., a protein with an amino acid sequence selected from the group consisting of SEQ ID NO:2, 4, 6, 8, 10, 12, 14, 16, 18, 20, 22 and 24. Alternatively, an antibody can detect an altered protein with a genetically altered TET2, RUNX1, NRAS, KRAS, DNMT3A, TP53, IDH2, EZH2, IDH1, NPM1, PHF6 or ASXL1 protein, e.g., a protein with an amino acid sequence selected from the group consisting of a mutant of SEQ ID NO: 2, 4, 6, 8, 10, 12, 14, 16, 18, 20, 22 and 24. Residues translated from codons listed as altered in Table 1 or in public databases such as COSMIC of dbGaP can be prepared in immunogenic compositions for generation of antibodies that will specifically recognize and bind to the mutant residues. Another method can employ pairs of antibodies, wherein one of the pair would bind a marker protein upstream, i.e., N-terminal to the region of expected alteration, e.g., nonsense or deletion and the other of the pair would bind the protein downstream. Wild type protein would bind both antibodies of the pair, but a protein with a nonsense or deletion alteration would bind only the N-terminal antibody of the pair. An assay such as a sandwich ELISA assay could detect a loss of quantity of the wild type protein in the tumor sample, e.g., in comparison to the reference sample, or a standard ELISA would permit comparison of the levels of binding of the antibodies to infer that an alteration is present in a tumor sample.

In some embodiments, indirect methods for determining the amount or functionality of a protein marker also include measurement of the activity of the protein. For example, a sample, or a protein isolated from the sample or expressed from nucleic acid isolated, cloned or amplified from the sample can be assessed for marker protein activity. The enzymatic activity of TET2 to generate 5-hydroxymethylcytosine on DNA can be measured (Shen and Zhang (2012)Methods Enzymol. 512: 93-105), the enzymatic activity of DNMT3A on DNA can be measured (Suetake et al. (2003) J. Biochem. 133:737-744) or the enzymatic activity of IDH1 or IDH2 can be measured (Murugan et al. (2011) Biochem. Biophys. Res. Comm. 393:555-559), e.g., in a tumor cell sample. In another example, for a RAS oncogene (KRAS or NRAS) an activating alteration can be measured as reduced GTPase activity or altered binding to RasGAP or a cell membrane in a cell-free assay (e.g., Shubbert et al. (2007) Mol. Cell Biol. 27:7765-7770). In another example, the phosphorylation state of NPM1 can be measured. The binding of RUNX1 to DNA at a RUNX1-binding element, e.g., in a gel shift assay or in a reporter assay (Michaud et al. (2002) Blood 99:1364-1372) can be measured. In another example the activity of ASXL1 can be measured in a retinoic acid reporter assay (Cho et al. (2006) J. Biol. Chem. 281:17588-17598). In another example, TP53 activity can be measured by the ability to bind to DNA or to form tetramers.

Another method for determining the level of a polypeptide corresponding to a marker is mass spectrometry. For example, intact proteins or peptides, e.g., tryptic peptides can be analyzed from a biological sample, e.g., a bone marrow sample, a blood sample, a lymph sample or other sample, containing one or more polypeptide markers. The method can further include treating the sample to lower the amounts of abundant proteins, e.g., serum albumin, to increase the sensitivity of the method. For example, liquid chromatography can be used to fractionate the sample so portions of the sample can be analyzed separately by mass spectrometry. The steps can be performed in separate systems or in a combined liquid chromatography/mass spectrometry system (LC/MS, see for example, Liao, et al. (2004) Arthritis Rheum. 50:3792-3803). The mass spectrometry system also can be in tandem (MS/MS) mode. The charge state distribution of the protein or peptide mixture can be acquired over one or multiple scans and analyzed by statistical methods, e.g. using the retention time and mass-to-charge ratio (m/z) in the LC/MS system, to identify proteins expressed at statistically significant levels differentially in samples from patients responsive or non-responsive to NAE inhibition therapy. Examples of mass spectrometers which can be used are an ion trap system (ThermoFinnigan, San Jose, Calif.) or a quadrupole time-of-flight mass spectrometer (Applied Biosystems, Foster City, Calif.). The method can further include the step of peptide mass fingerprinting, e.g. in a matrix-assisted laser desorption ionization with time-of-flight (MALDI-TOF) mass spectrometry method. The method can further include the step of sequencing one or more of the tryptic peptides. Results of this method can be used to identify proteins from primary sequence databases, e.g., maintained by the National Center for Biotechnology Information, Bethesda, Md., or the Swiss Institute for Bioinformatics, Geneva, Switzerland, and based on mass spectrometry tryptic peptide m/z base peaks.

In one embodiment, expression of a marker is assessed by preparing mRNA/cDNA (i.e., a transcribed polynucleotide) from cells in a biological sample, and by hybridizing the mRNA/cDNA with a reference polynucleotide, e.g., an isolated nucleic acid probe, e.g., a hybridization probe, which is a complement of a marker nucleic acid, or a fragment thereof. cDNA can, optionally, be amplified using any of a variety of polymerase chain reaction methods prior to hybridization with the reference polynucleotide. Expression of one or more markers likewise can be detected using quantitative PCR to assess the level of expression of the marker(s). An example of the use of measuring mRNA levels is that an inactivating alteration in a marker gene can result in an altered level of mRNA in a cell. The level can be upregulated due to feedback signaling protein production in view of nonfunctional or absent protein or downregulated due to instability of an altered mRNA sequence. Alternatively, any of the many known methods of detecting alterations (e.g. single nucleotide polymorphisms, deletions, discussed above) of a marker of the invention may be used to detect occurrence of an alteration in a marker gene in a patient.

In some embodiments direct measurement of nucleic acid amount is quantification of transcripts. As used herein, the level or amount of expression refers to the absolute amount of expression of an mRNA encoded by the marker or the absolute amount of expression of the protein encoded by the marker. As an alternative to making determinations based on the absolute expression amount of selected markers, determinations may be based on normalized expression amounts. Expression amount can be normalized by correcting the absolute expression level of a marker upon comparing its expression to the expression of a control marker that is not a marker, e.g., in a housekeeping role that is constitutively expressed. Suitable markers for normalization also include housekeeping genes, such as the actin gene or beta-2 microglobulin. Reference markers for data normalization purposes include markers which are ubiquitously expressed and/or whose expression is not regulated by oncogenes. Constitutively expressed genes are known in the art and can be identified and selected according to the relevant tissue and/or situation of the patient and the analysis methods. Such normalization allows one to compare the expression level in one biological sample, to another biological sample, e.g., between biological samples from different times or different subjects. Further, the expression level can be provided as a relative expression level. The baseline of a genomic DNA sample, e.g., diploid copy number, can be determined by measuring amounts in cells from subjects without a tumor or in non-tumor cells from the patient. To determine a relative amount of a marker or marker set, the amount of the marker or marker set is determined for at least 1, or 2, 3, 4, 5, or more samples, e.g., 7, 10, 15, 20 or 50 or more samples in order to establish a baseline, prior to the determination of the expression level for the sample in question. To establish a baseline measurement, the mean amount or level of each of the markers or marker sets assayed in the larger number of samples is determined and this is used as a baseline expression level for the biomarkers or biomarker sets in question. The amount of the marker or marker set determined for the test sample (e.g., absolute level of expression) is then divided by the baseline value obtained for that marker or marker set. This provides a relative amount and aids in identifying abnormal levels of marker protein activity.

Probes based on the sequence of a nucleic acid molecule of the invention can be used to detect transcripts or genomic sequences corresponding to one or more markers of the invention. The probe can comprise a label group attached thereto, e.g., a radioisotope, a fluorescent compound, an enzyme, or an enzyme co-factor. Probes can be used as part of a diagnostic test kit for identifying cells or tissues which express the protein, such as by measuring levels of a nucleic acid molecule encoding the protein in a sample of cells from a subject, e.g., detecting mRNA levels or determining whether a gene encoding the protein has been altered.

Primers or nucleic acid probes comprise a nucleotide sequence complementary to a specific a marker or an altered region thereof and are of sufficient length to selectively bind or hybridize with a marker gene or nucleic acid associated with a marker gene, e.g., they can bind to the nucleic acid with base sequence specificity and remain bound, after washing. Primers and probes can be used to aid in the isolation and sequencing of marker nucleic acids. In one embodiment, the primer or nucleic acid probe, e.g., a substantially purified oligonucleotide, an isolated nucleic acid, comprises a region having a nucleotide sequence which binds, e.g., hybridizes, e.g., under stringent conditions, to about 5 to 15, 10 to 25, 15 to 50, 20 to 100, 50 to 350 or 500 or more consecutive nucleotides of a marker gene or a region comprising an alteration in a marker gene or transcript therefrom or a complement thereof. In another embodiment, the primer or nucleic acid probe is capable of hybridizing to a marker nucleic acid corresponding to a marker gene described herein, e.g., TET2, RUNX1, NRAS, KRAS, DNMT3A, TP53, IDH2, EZH2, IDH1, NPM1, PHF6, or ASXL1, e.g., a nucleic acid comprising a nucleotide sequence of any sequence set forth in any of SEQ ID NOs:1, 3, 5, 7, 9, 11, 13, 15, 17, 19, 21, 23 or a sequence on a chromosome locus listed in Table 1, or a complement of any of the foregoing. For example, a primer or nucleic acid probe comprising a nucleotide sequence of at least about 10 consecutive nucleotides, at least about 15 consecutive nucleotides, about 10 to 25 consecutive nucleotides, about 20 to 40 consecutive nucleotides, about 30 to 60 consecutive nucleotides, or having from about 15 to about 30 nucleotides set forth in any of SEQ ID NOs: 1, 3, 5, 7, 9, 11, 13, 15, 17, 19, 21, 23, an open reading frame of SEQ ID NO: 1, 3, 5, 7, 9, 11, 13, 15, 17, 19, 21, 23, or a sequence on a chromosome locus listed in Table 1, or a complement of any of the foregoing are provided by the invention. Primers or nucleic acid probes having a sequence of more than about 25, 40 or 50 nucleotides are also within the scope of the invention. In another embodiment, a primer or nucleic acid probe can have a sequence at least 70%, at least 75%, 80% or 85%, or at least, 90% 95% or 97% identical to the nucleotide sequence of any sequence set forth in any of SEQ ID NOs: 1, 3, 5, 7, 9, 11, 13, 15, 17, 19, 21, 23, an open reading frame of SEQ ID NO: 1, 3, 5, 7, 9, 11, 13, 15, 17, 19, 21, 23, or a sequence on a chromosome locus listed in Table 1, or a complement of any of the foregoing. Nucleic acid analogs can be used as binding sites for hybridization. An example of a suitable nucleic acid analogue is peptide nucleic acid (see, e.g., Egholm et al., Nature 363:566 568 (1993); U.S. Pat. No. 5,539,083).

In some embodiments, a nucleic acid probe can be designed to bind to the wild type sequence, so the presence of an alteration in that region can cause a decrease, e.g., measurable decrease, in binding or hybridization by that probe. In another embodiment, a nucleic acid probe can be designed to bind to a mutant sequence, so the presence of an alteration in that region can cause an increase in binding or hybridization by that probe. In other embodiments, a probe and primer set or a primer pair can be designed to bracket a region in a marker that can have an alteration so amplification based on that set or pair can result in nucleic acids which can be sequenced to identify the alteration, as described above.

Primers or nucleic acid probes can be selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001). Useful primers or nucleic acid probes of the invention bind sequences which are unique for each transcript, e.g., target altered regions and can be used in PCR for amplifying, detecting and sequencing only that particular nucleic acid, e.g., transcript or altered transcript. Examples of some portions, e.g., codons, of marker genes, e.g., TET2, RUNX1, NRAS, KRAS, and/or DNMT3A TP53, IDH2, EZH2, IDH1, NPM1, PHF6, and/or ASXL1, which may be altered in cancer, e.g., hematological cancer, are found in Table 1. Other alterations are described in reference articles cited herein and in public databases described herein. One of skill in the art can design primers and nucleic acid probes for the markers disclosed herein or related markers with similar characteristics, e.g., markers on the chromosome loci, or alterations in different regions of the same marker gene described herein, using the skill in the art, e.g., adjusting the potential for primer or nucleic acid probe binding to standard sequences, mutants or allelic variants by manipulating degeneracy or GC content in the primer or nucleic acid probe. Computer programs that are well known in the art are useful in the design of primers with the required specificity and optimal amplification properties, such as Oligo version 5.0 (National Biosciences, Plymouth, Minn.). While perfectly complementary nucleic acid probes and primers can be used for detecting the markers described herein and mutants, polymorphisms or alleles thereof, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the nucleic acid probe or primer as long as the resulting probe or primer is still capable of specifically hybridizing to the target region.

An indication of treatment outcome can be assessed by studying a characteristic of 1 marker, characteristics of markers in a marker set comprising 2 markers, 3 markers or 4 markers, or more, e.g., 5, 6, 7, 8, 9, 10, 15, 20, or 25 markers, or altered portions thereof e.g., marker genes which interact with DNA, signaling pathways or are involved in AML tumorigenesis. Markers can be studied in combination with another measure of treatment outcome, e.g., biochemical markers (e.g., M protein level in myeloma, kidney health marker such as proteinuria, serum levels of C-reactive protein or cytokeratin 19, cytokeratin fragment 21-1 (CYFRA21-1) for NSCLC, urine levels of fibrinogen/fibrinogen degradation products for bladder cancer, urine or blood levels of catecholamines for neuroblastoma, serum levels of carbohydrate antigen 19-9 (CA 19-9) or metabolic profiling for pancreatic cancer or blood levels of soluble mesothelin-related peptides (SMRP) in mesothelioma) or histology assessment (e.g., fewer than 5% blast cells in the bone marrow of a leukemia or myeloma patient, number of mitotic figures per unit area, depth measurement of invasion of melanoma tumors, esophageal tumors or bladder tumors).

Statistical methods can assist in the determination of treatment outcome upon measurement of a characteristic such as an amount of a marker, e.g., measurement of DNA, RNA or protein. The amount of one marker can be measured at multiple timepoints, e.g., before treatment, during treatment, after treatment with an agent, e.g., an NAE inhibitor. To determine the progression of change in expression of a marker from a baseline, e.g., over time, the expression results can be analyzed by a repeated measures linear regression model (Littell, Miliken, Stroup, Wolfinger, Schabenberger (2006) SAS for Mixed Models, 2^{nd} edition. SAS Institute, Inc., Cary, N.C.)):

Yᵢⱼₖ−Yᵢⱼ₀=Yᵢⱼ₀+treatmentᵢ+dayₖ+(treatment*day)ᵢ_{ₖ}+εᵢⱼₖ Equation 1

where Yᵢⱼₖ is the log 2 transformed expression (normalized to the housekeeping genes) on the k^{th} day of the j^{th} animal in the i^{th} treatment, Yᵢⱼ₀ is the defined baseline log₂ transformed expression (normalized to the housekeeping genes) of the j^{th} animal in the i^{th} treatment, dayₖ is treated as a categorical variable, and εᵢⱼₖ is the residual error term. A covariance matrix (e.g., first-order autoregressive, compound symmetry, spatial power law) can be specified to model the repeated measurements on each animal over time. Furthermore, each treatment time point can be compared back to the same time point in the vehicle group to test whether the treatment value was significantly different from vehicle.

A number of other methods can be used to analyze the data. For instance, the relative expression values could be analyzed instead of the cycle number. These values could be examined as either a fold change or as an absolute difference from baseline. Additionally, a repeated-measures analysis of variance (ANOVA) could be used if the variances are equal across all groups and time points. The observed change from baseline at the last (or other) time point could be analyzed using a paired t-test, a Fisher exact test (p-value=ΣP(X=x) from x=1 to the number of situations, e.g., alterations, tested that show sensitivity to NAE inhibition) for testing significance of data of small sample sizes, or a Wilcoxon signed rank test if the data is not normally distributed, to compare whether a cancer patient was significantly different from a normal subject.

A difference in amount from one timepoint to the next or from the tumor sample to the normal sample can indicate prognosis or treatment outcome. A baseline level can be determined by measuring expression at 1, 2, 3, 4, or more times prior to treatment, e.g., at time zero, one day, three days, one week and/or two weeks or more before treatment. Alternatively, a baseline level can be determined from a number of subjects, e.g., normal subjects or patients with the same health status or disorder, who do not undergo or have not yet undergone the treatment, as discussed above. Alternatively, one can use expression values deposited with the Gene Expression Omnibus (GEO) program at the National Center for Biotechnology Information (NCBI, Bethesda, Md.). For example, datasets of myeloma mRNA expression amounts sampled prior to proteasome inhibition therapy include GEO Accession number GSE9782, also analyzed in Mulligan, et al. (2006) Blood 109:3177-88 and GSE6477, also analyzed by Chng et al. (2007) Cancer Res. 67:292-9. To test the effect of the treatment on the tumor, the expression of the marker can be measured at any time or multiple times after some treatment, e.g., after 1 day, 2 days, 3 days, 5 days, 1 week, 2 weeks, 3 weeks, 4 weeks, 1 month, 2 months, 3 months and/or 6 or more months of treatment. For example, the amount of a marker can be measured once after some treatment, or at multiple intervals, e.g., 1-week, 2-week, 4-week or 2-month, 3-month or longer intervals during treatment. In some embodiments, the measurement of a marker during treatment can be compared to the same marker measurement at baseline. In other embodiments, the measurement of a marker during treatment can be compared to the same marker measurement at an earlier timepoint. Conversely, to determine onset of progressive disease after stopping the administration of a therapeutic regimen, the amount of the marker can be measured at any time or multiple times after, e.g., 1 day, 2 days, 3 days, 5 days, 1 week, 2 weeks, 3 weeks, 4 weeks, 1 month, 2 months, 3 months and/or 6 or more months after the last treatment. The measurement of a marker after treatment can be compared to the same marker measurement at the end of treatment. One of skill in the art would determine the timepoint or timepoints to assess the amount of the marker depending on various factors, e.g., the pharmacokinetics of the treatment, the treatment duration, pharmacodynamics of the treatment, age of the patient, the nature of the disorder or mechanism of action of the treatment. A trend in the negative direction or a decrease in the amount relative to baseline or a pre-determined standard of expression of a marker of sensitivity to NAE inhibition therapy can indicate a decrease in response of the tumor to the therapy, e.g., increase in resistance. A trend toward a favorable outcome relative to the baseline or a pre-determined standard of expression of a marker of treatment outcome indicates usefulness of the therapeutic regimen or continued benefit of the therapy.

Any marker, e.g., nucleic acid or protein corresponding to a marker gene or combination of marker of the invention, or alterations thereof as well as any known markers in combination with the markers of the invention, may be used in the compositions, kits, and methods of the present invention. In general, markers are selected for as great as possible ability to judge mutational status of a marker gene to predict outcome of treatment with a therapeutic regimen comprising an NAE inhibitor. For example, the choice of markers are selected for as great as possible difference between the characteristic, e.g., size, sequence, composition or amount of the marker in samples comprising tumor cells and the characteristic, e.g., size, sequence, composition or amount of the same marker in control cells. Although this difference can be as small as the limit of detection of the method for assessing the amount of the marker, in another embodiment, the difference can be at least greater than the standard error of the assessment method. In the case of RNA or protein amount, a difference can be at least 1.5-, 2-, 3-, 4-, 5-, 6-, 7-, 8-, 9-, 10-, 15-, 20-, 25-, 100-, 500-, 1000-fold or greater. “Low” RNA or protein amount can be that expression relative to the overall mean across tumor samples (e.g., from a hematological tumor, e.g., leukemia or myeloma) is low. In the case of amount of DNA, e.g., copy number, the amount is 0, 1, 2, 3, 4, 5, 6, or more copies. A deletion causes the copy number to be 0 or 1; an amplification causes the copy number to be greater than 2. The difference can be qualified by a confidence level, e.g., p<0.05, p<0.02, p<0.01 or lower p-value.

Measurement of more than one marker, e.g., a marker set of 2, 3, 4, 2 to 5, 5, 6, 7, 8, 9, 4 to 10, 10, 12, 15, 8 to 20, 20, or 25 or more markers, can provide a profile, e.g., for amounts of mRNA, an expression profile or a trend indicative of treatment outcome. In some embodiments, the marker set comprises no more than 2, 3, 4, 2 to 5, 5, 6, 7, 8, 9, 4 to 10, 10, 12, 15, 8 to 20, 20, or 25 markers. In some embodiments, the marker set includes a plurality of chromosome loci, a plurality of marker genes, or a plurality of markers of one or more marker genes (e.g., nucleic acid and protein, genomic DNA and mRNA, or various combinations of markers described herein). Analysis of treatment outcome through assessing the amount of markers in a set can be accompanied by a statistical method, e.g., a weighted voting analysis which accounts for variables which can affect the contribution of the amount of a marker in the set to the class or trend of treatment outcome, e.g., the signal-to-noise ratio of the measurement or hybridization efficiency for each marker. A marker set, e.g., a set of 2, 3, 4, 2 to 5, 5, 6, 7, 8, 9, 4 to 10, 10, 12, 15, 8 to 20, 20, or 25 or more markers, can use a primer, probe or primers to analyze at least one marker nucleic acid, e.g., DNA or RNA described herein, e.g., a marker on a chromosome locus listed in Table 1, a nucleic acid of a marker gene such as TET2, RUNX1, NRAS, KRAS, DNMT3A, TP53, IDH2, EZH2, IDH1, NPM1, PHF6, and/or ASXL1, or a complement of any of the foregoing. A marker set, e.g., a set of at least 2, 3, 4, 2 to 5, 5, 6, 7, 8, 9, 4 to 10, 10, 12, 15, 8 to 20, 20, or 25 or more markers, can use a primer, probe or primers to detect at least one or at least 2, 3, 4, 2 to 5, 5, 6, 7, 8, 9, 4 to 10, 10, 12, 15, 8 to 20, 20, or 25 or more alterations on the markers e.g., of TET2, RUNX1, NRAS, KRAS, DNMT3A, TP53, IDH2, EZH2, IDH1, NPM1, PHF6, and/or ASXL1.

In one embodiment, a marker set can be used for assessing mutational status of a marker gene selected from the group consisting of TET2, RUNX1, NRAS, KRAS and DNMT3A and further be used for assessing mutational status of a marker gene selected from the group consisting of TP53, IDH2, EZH2, IDH1, NPM1, PHF6, and ASXL1. In another embodiment, a marker set can comprise markers for assessing characteristics of ASXL1 and IDH1. In an embodiment, a marker set comprising at least one marker for assessing at least one characteristic of ASXL1 and IDH1 further comprises at least one marker for assessing at least one characteristic of a marker selected from the group consisting of TET2, RUNX1, NRAS, KRAS, DNMT3A and TP53. In an embodiment, a marker set comprising at least one marker for assessing at least one characteristic of ASXL1 and IDH1 further comprises at least one marker for assessing at least one characteristic of a marker selected from the group consisting of TET2, RUNX1, NRAS, KRAS, DNMT3A, TP53, IDH2, EZH2, NPM1 and PHF6. In some embodiments, a marker set identifies wild type ASXL1 and IDH1. In some embodiments, a marker set identifies wild type ASXL1 and IDH1 and further identifies mutant TET2, RUNX1, NRAS, KRAS, DNMT3A and/or TP53.

Selected marker sets can be assembled from the markers provided herein or selected from among markers using methods provided herein and analogous methods known in the art. A way to qualify a new marker for use in an assay of the invention is to correlate a characteristic, e.g., size, sequence, composition or amount in a sample comprising tumor cells with differences in characteristic, e.g., size, sequence, composition or amount (e.g., fold-change from baseline) of a marker, e.g., a marker gene. A useful way to judge the relationship is to calculate the coefficient of determination r2, after solving for r, the Pearson product moment correlation coefficient and/or preparing a least squares plot, using standard statistical methods. For example, a correlation can analyze DNA copy number versus the level of expression of marker, e.g., a marker gene. A gene product can be selected as a marker if the result of the correlation (r2, e.g., the linear slope of the data in this analysis), is at least 0.1-0.2, at least 0.3-0.5, or at least 0.6-0.8 or more. Markers can vary with a positive correlation to response, PFS or survival (i.e., change expression levels in the same manner as copy number, e.g., decrease when copy number is decreased). Markers which vary with a negative correlation to copy number (i.e., change expression levels in the opposite manner as copy number levels, e.g., increase when copy number is decreased) provide inconsistent determination of outcome. In another embodiment, marker set can be prepared using a scoring method known in the art (e.g., weighted voting, combination of threshold features (CTF), Cox proportional hazards analysis, principal components scoring, linear predictive score, K-nearest neighbor, etc.), e.g., using expression values deposited with the Gene Expression Omnibus (GEO) program at the National Center for Biotechnology Information (NCBI, Bethesda, Md.).

In embodiments when the compositions, kits, and methods of the invention are used for characterizing treatment outcome in a patient, the marker or set of markers of the invention is selected such that a significant result is obtained in at least about 20%, at least about 40%, 60%, or 80%, or in substantially all patients treated with the test agent. The marker or set of markers of the invention can be selected such that a positive predictive value (PPV) of greater than about 10% is obtained for the general population and additional confidence in a marker can be inferred when the PPV is coupled with an assay specificity greater than 80%.

In an example wherein treatment with a regimen comprising NAE inhibitor, e.g., pevonedistat or a pharmaceutically acceptable salt thereof comprises measuring the amount of mRNA, an expression level in a sample comprising tumor cells from the patient is measured for a marker corresponding to at least one of the markers described herein. A marker set can be utilized wherein the marker set has the following properties: it includes markers of a plurality of marker genes, each of which is differentially expressed as between patients with identified outcome and non-afflicted subjects and it contains a sufficient number of differentially expressed markers such that differential amount (e.g., as compared to a level in a non-afflicted reference sample) of each of the markers in the marker set in a subject is predictive of treatment outcome with no more than about 15%, about 10%, about 5%, about 2.5%, or about 1% false positives, and assayed using the methods described herein. Such analysis is used to obtain an expression profile of the tumor in the patient. Evaluation of the expression profile is then used to determine whether the patient is expected to have a favorable outcome and would benefit from treatment, e.g., treatment with the NAE inhibitor alone, or in combination with additional agents, e.g., a hypomethylating agent, e.g., azacitidine)), or an alternative agent expected to have a similar effect on survival. Evaluation of the expression profile can also be used to determine whether a patient is expected to have an unfavorable outcome and would benefit from a cancer therapy other than NAE inhibition therapy or would benefit from an altered NAE inhibition therapy regimen. A patient whose cancer is characterized as having a marker profile indicative of favorable outcome to NAE inhibition therapy, e.g., pevonedistat or a pharmaceutically acceptable salt thereof, will undergo treatment with the therapy or a more aggressive therapy regimen will be identified for a patient with an expected unfavorable outcome.

Examining the amount of one or more of the identified markers or marker sets in a tumor sample taken from a patient during the course of NAE inhibition therapy, pevonedistat or a pharmaceutically acceptable salt thereof, it is also possible to determine whether the therapeutic agent is continuing to work or whether the cancer has become non-responsive (refractory) to the treatment protocol. For example, a patient receiving a treatment of pevonedistat or a pharmaceutically acceptable salt thereof would have tumor cells removed and monitored for the expression of a marker or marker set. If the profile of the amount of one or more markers identified herein more typifies favorable outcome in the presence of the agent, e.g., the NAE inhibitor, e.g. pevonedistat or a pharmaceutically acceptable salt thereof, the treatment would continue. However, if the profile of the amount of one or more markers identified herein more typifies unfavorable outcome in the presence of the agent, then the cancer may have become resistant to therapy, e.g., NAE inhibition therapy, and another treatment protocol should be initiated to treat the patient. For example, the cancer, e.g., a hematological cancer, may comprise an alteration in a marker gene associated with resistance to NAE inhibition.

### Therapeutic Agents

The markers and marker sets of the present invention assess the likelihood of favorable outcome of therapy (e.g., sensitivity to a therapeutic agent) in patients, e.g., patients having cancer, e.g., hematological cancer, such as leukemia, lymphoma or myeloma (e.g., acute myelogenous leukemia, myelodysplastic syndrome or chronic myelomonocytic leukemia), based on a characteristic, e.g., size, sequence, composition or amount of a marker or markers of the invention. Using this prediction, the patient can be treated with a therapy regimen best suitable for a favorable outcome.

In particular, the methods characterize patient cancer treatment outcome with a regimen comprising an NAE inhibitor as described in earlier sections. The agents provided in the present methods can be a single agent or a combination of agents. The methods of the invention include combination of NAE inhibition therapy with hypomethylating agent therapy, such as azacitidine, and/or other or additional agents, e.g., selected from the group consisting of chemotherapeutic agents. For example, the present methods can be used to determine whether a single chemotherapeutic agent, such as an NAE inhibitor, e.g., pevonedistat or a pharmaceutically acceptable salt thereof can be used to treat a cancer or whether one or more agents should be used in combination with the NAE inhibitor (e.g., pevonedistat or a pharmaceutically acceptable salt thereof). Useful combinations can include agents that have different mechanisms of action than the NAE inhibitor, e.g., an anti-mitotic agent, an alkylating agent, an antimetabolite, or a proteasome inhibitor. In some embodiments, the methods provide for a therapeutic regimen comprising pevonedistat or a pharmaceutically acceptable salt thereof and a hypomethylating agent, e.g. azacitidine. In some embodiments, the methods provide for a therapeutic regimen comprising pevonedistat or a pharmaceutically salt thereof and azacitidine.

As used herein, the term “proteasome inhibitor” refers to any substance which directly inhibits enzymatic activity of the 20S or 26S proteasome in vitro or in vivo. Examples of proteasome inhibitors are bortezomib, carfilzomib, ixazomib, disulfiram, epigallocatechin-3-gallate, salinosporamid A, ONX0912, CEP-18770, and epoxomicin.

Other therapeutic agents for use in combination with NAE inhibition therapy include chemotherapeutic agents. A “chemotherapeutic agent” is intended to include chemical reagents which inhibit the growth of proliferating cells or tissues wherein the growth of such cells or tissues is undesirable. Chemotherapeutic agents used in the treatment of hematological cancer, such as leukemia, e.g., AML, MDS or CMML include antibiotics, e.g., daunorubicin, adriamycin or idarubicin, or antimetabolites, e.g., pyrimidine analogs, e.g., cytarabine or gemcitabine, and are well known in the art (see e.g., Gilman A.G., et al., The Pharmacological Basis of Therapeutics, 8th Ed., Sec 12:1202-1263 (1990)), and are typically used to treat neoplastic diseases.

In some embodiments, the patient with cancer, e.g., a solid tumor, is administered a combination of pevonedistat or a pharmaceutically acceptable salt thereof and one or more chemotherapeutic agents, wherein the chemotherapeutic agent is one or more of: (i) a taxane; (ii) a platin; or (iii) gemcitabine. The taxane, platin, and/or gemcitabine are administered in standard doses.

Taxane agents include paclitaxel and docetaxel. In some embodiments, the taxane is paclitaxel, docetaxel or nab-paclitaxel. In some embodiments, the taxane is paclitaxel or docetaxel. In some embodiments, the taxane is paclitaxel. In some embodiments, the taxane is docetaxel.

In some embodiments, the platin is cisplatin, carboplatin, oxaliplatin, satraplatin, picoplatin, nedaplatin or triplatin. In some embodiments, the platin is nedaplatin, cisplatin, carboplatin or oxaliplatin. In some embodiments, the platin is cisplatin, carboplatin or oxaliplatin. In some embodiments, the platin is cisplatin. In some embodiments, the platin is carboplatin. In some embodiments, the platin is cisplatin or carboplatin.

In some embodiments, pevonedistat or a pharmaceutically acceptable salt thereof is administered on each of days 1, 3, and 5 of a 21 day schedule. In some embodiments, the amount of pevonedistat or a pharmaceutically acceptable salt thereof that is administered on each of days 1, 3, and 5 of a 21 day schedule is less than or equal to 50 mg/m². In some embodiments, the amount of pevonedistat or a pharmaceutically acceptable salt thereof that is administered on each of days 1, 3, and 5 of a 21 day schedule is about 50 mg/m². In some embodiments, the amount of pevonedistat or a pharmaceutically acceptable salt thereof that is administered on each of days 1, 3, and 5 of a 21 day schedule is about 37 mg/m². In some embodiments, the amount of pevonedistat or a pharmaceutically acceptable salt thereof that is administered on each of days 1, 3, and 5 of a 21 day schedule is about 25 mg/m². In some embodiments, the amount of pevonedistat or a pharmaceutically acceptable salt thereof that is administered on each of days 1, 3, and 5 of a 21 day schedule is about 15 mg/m². In some embodiments, the amount of pevonedistat or a pharmaceutically acceptable salt thereof that is administered on each of days 1, 3, and 5 of a 21 day schedule is about 20 mg/m². In some embodiments, the amount of pevonedistat or a pharmaceutically acceptable salt thereof that is administered on each of days 1, 3, and 5 of a 21 day schedule is about 10 mg/m² to about 30 mg/m². In some embodiments, the amount of pevonedistat or a pharmaceutically acceptable salt thereof that is administered on each of days 1, 3, and 5 of a 21 day schedule is about 15 mg/m² to about 45 mg/m².

In some embodiments, pevonedistat or a pharmaceutically acceptable salt thereof is administered on each of days 1, 8, and 15 of a 28 day schedule. In some embodiments, the amount of pevonedistat or a pharmaceutically acceptable salt thereof that is administered on each of days 1, 8, and 15 of a 28 day schedule is less than or equal to 100 mg/m². In some embodiments, the amount of pevonedistat or a pharmaceutically acceptable salt thereof that is administered on each of days 1, 8, and 15 of a 28 day schedule is about 100 mg/m². In some embodiments, the amount of pevonedistat or a pharmaceutically acceptable salt thereof that is administered on each of days 1, 8, and 15 of a 28 day schedule is about 75 mg/m². In some embodiments, the amount of pevonedistat or a pharmaceutically acceptable salt thereof that is administered on each of days 1, 8, and 15 of a 28 day schedule is about 50 mg/m². In some embodiments, the amount of pevonedistat or a pharmaceutically acceptable salt thereof that is administered on each of days 1, 8, and 15 of a 28 day schedule is about 25 mg/m². In some embodiments, the amount of pevonedistat or a pharmaceutically acceptable salt thereof that is administered on each of days 1, 8, and 15 of a 28 day schedule is about 20 mg/m². In some embodiments, the amount of pevonedistat or a pharmaceutically acceptable salt thereof that is administered on each of days 1, 8, and 15 of a 28 day schedule is about 15 mg/m². In some embodiments, the amount of pevonedistat or a pharmaceutically acceptable salt thereof that is administered on each of days 1, 8, and 15 of a 28 day schedule is about 15 mg/m² to about 40 mg/m².

In some embodiments, pevonedistat or a pharmaceutically acceptable salt thereof is administered on day 1 of a 21 day schedule. In some embodiments, the amount of pevonedistat or a pharmaceutically acceptable salt thereof that is administered on day 1 of a 21 day schedule is less than or equal to 50 mg/m². In some embodiments, the amount of pevonedistat or a pharmaceutically acceptable salt thereof that is administered on day 1 of a 21 day schedule is less than or equal to 25 mg/m². In some embodiments, the amount of pevonedistat or a pharmaceutically acceptable salt thereof that is administered on day 1 of a 21 day schedule is 20 mg/m². In some embodiments, the amount of pevonedistat or a pharmaceutically acceptable salt thereof that is administered on day 1 of a 21 day schedule is less than or equal to 15 mg/m². In some embodiments, pevonedistat or a pharmaceutically acceptable salt thereof is administered on day 1 of a 28 day schedule. In some embodiments, the amount of pevonedistat or a pharmaceutically acceptable salt thereof that is administered on day 1 of a 28 day schedule is less than or equal to 100 mg/m².

The agents disclosed herein may be administered by any route, including intradermally, subcutaneously, orally, intraarterially or intravenously. In one embodiment, administration will be by the intravenous route. Parenteral administration can be provided in a bolus or by infusion.

In some embodiments, pevonedistat or a pharmaceutically acceptable salt thereof is administered in combination with azacitidine. In some embodiments, pevonedistat or a pharmaceutically acceptable salt thereof was administered via a 60-minute intravenous (IV) infusion on days 1, 3, and 5 in escalating doses beginning at 20 mg/m² and azacitidine is administered via IV at a dose of 75 mg/m² on days 1-5, 8, and 9.

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. Treatments may be administered daily or more frequently depending upon a number of factors, including the overall health of a patient, and the formulation and route of administration of the selected compound(s).

### Electronic Apparatus Readable Arrays

Electronic apparatus, including readable arrays comprising at least one predictive marker of the present invention is also contemplated for use in conjunction with the methods of the invention. As used herein, “electronic apparatus readable media” refers to any suitable medium for storing, holding or containing data or information that can be read and accessed directly by an electronic apparatus. As used herein, the term “electronic apparatus” is intended to include any suitable computing or processing apparatus or other device configured or adapted for storing data or information. Examples of electronic apparatus suitable for use with the present invention and monitoring of the recorded information include stand-alone computing apparatus; networks, including a local area network (LAN), a wide area network (WAN) Internet, Intranet, and Extranet; electronic appliances such as personal digital assistants (PDAs), cellular phone, pager and the like; and local and distributed processing systems. As used herein, “recorded” refers to a process for storing or encoding information on the electronic apparatus readable medium. Those skilled in the art can readily adopt any of the presently known methods for recording information on known media to generate manufactures comprising the markers of the present invention.

For example, microarray systems are well known and used in the art for assessment of samples, whether by assessment gene expression (e.g., DNA detection, RNA detection, protein detection), or metabolite production, for example. Microarrays for use according to the invention include one or more probes of predictive marker(s) of the invention characteristic of response and/or non-response to a therapeutic regimen as described herein. In one embodiment, the microarray comprises one or more probes corresponding to one or more of markers selected from the group consisting of markers which demonstrate increased expression in short term survivors, and genes which demonstrate increased expression in long term survivors in patients. A number of different microarray configurations and methods for their production are known to those of skill in the art and are disclosed, for example, in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 5,981,185; 6,022,963; 6,077,674; 6,156,501; 6,261,776; 6,346,413; 6,440,677; 6,451,536; 6,576,424; 6,610,482; 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and U.S. Pat. No. 5,874,219; Shena, et al. (1998), Tibtech 16:301; Duggan et al. (1999) Nat. Genet. 21:10; Bowtell et al. (1999) Nat. Genet. 21:25; Lipshutz et al. (1999) Nature Genet. 21:20-24, 1999; Blanchard, et al. (1996) Biosensors and Bioelectronics, 11:687-90; Maskos, et al., (1993) Nucleic Acids Res. 21:4663-69; Hughes, et al. (2001) Nat. Biotechol. 19:342, 2001; each of which are herein incorporated by reference. A tissue microarray can be used for protein identification (see Hans et al. (2004) Blood 103:275-282). A phage-epitope microarray can be used to identify one or more proteins in a sample based on whether the protein or proteins induce auto-antibodies in the patient (Bradford et al. (2006) Urol. Oncol. 24:237-242).

A microarray thus comprises one or more probes corresponding to one or more markers identified herein, e.g., those indicative of treatment outcome, e.g., to identify wild type marker genes, normal allelic variants and alterations of marker genes. The microarray can comprise probes corresponding to, for example, at least 2, at least 3, at least 4, at least 5, at least 10, at least 15, at least 20, at least 25, at least 30, at least 35, at least 40, at least 45, at least 50, at least 75, or at least 100, biomarkers and/or alterations thereof indicative of treatment outcome. The microarray can comprise probes corresponding to one or more biomarkers as set forth herein. Still further, the microarray may comprise complete marker sets as set forth herein and which may be selected and compiled according to the methods set forth herein. The microarray can be used to assay expression of one or more predictive markers or predictive marker sets in the array. In one example, the array can be used to assay more than one predictive marker or marker set expression in a sample to ascertain an expression profile of markers in the array. In this manner, up to about 44,000 markers can be simultaneously assayed for expression. This allows an expression profile to be developed showing a battery of markers specifically expressed in one or more samples. Still further, this allows an expression profile to be developed to assess treatment outcome.

The array is also useful for ascertaining differential expression patterns of one or more markers in normal and abnormal (e.g., sample, e.g., tumor) cells. This provides a battery of markers that could serve as a tool for ease of identification of treatment outcome of patients. Further, the array is useful for ascertaining expression of reference markers for reference expression levels. In another example, the array can be used to monitor the time course of expression of one or more markers in the array.

In addition to such qualitative determination, the invention allows the quantification of marker expression. Thus, predictive markers can be grouped on the basis of marker sets or outcome indications by the amount of the marker in the sample. This is useful, for example, in ascertaining the outcome of the sample by virtue of scoring the amounts according to the methods provided herein.

The array is also useful for ascertaining the effect of the expression of a marker on the expression of other predictive markers in the same cell or in different cells. This provides, for example, a selection of alternate molecular targets for therapeutic intervention if patient is predicted to have an unfavorable outcome.

### Reagents and Kits

The invention also encompasses kits for assaying a characteristic, e.g., size, sequence, composition or amount, of a marker, e.g., polypeptide or nucleic acid corresponding to a marker gene of the invention in a biological sample (e.g. a bone marrow sample, tumor biopsy or a reference sample). Such kits can be used, e.g., in the methods described herein, to determine mutational status of at least one marker gene for treating a patient who will experience a favorable outcome, e.g., after a treatment regimen comprising an NAE inhibitor, e.g., pevonedistat or a pharmaceutically acceptable salt thereof. For example, the kit can comprise a probe or reagent capable of detecting a genomic DNA segment, a polypeptide or a transcribed RNA corresponding to a marker of the invention or an alteration of a marker gene in a biological sample and means for determining the amount of the genomic DNA segment, the polypeptide or RNA in the sample. Suitable reagents for binding with a marker protein include antibodies, antibody derivatives, antibody fragments, and the like. Suitable reagents for binding with a marker nucleic acid (e.g., a genomic DNA, an mRNA, a spliced mRNA, a cDNA, or the like) include complementary nucleic acids. A label can be directly attached to the marker binding agent, e.g., probe, e.g., nucleic acid reagent such as a probe or primer or protein reagent, such as a specific binding agent or antibody, or a secondary reagent can comprise a label for indirect labeling. The kit can also contain a control or reference sample or a series of control or reference samples which can be assayed and compared to the test sample. For example, the kit may have a positive control sample, e.g., including one or more markers or alterations described herein, or reference markers, e.g. housekeeping markers to standardize the assay among samples or timepoints or reference genomes, e.g., form subjects without tumor e.g., to establish diploid copy number baseline or reference expression level of a marker. By way of example, the kit may comprise fluids (e.g., buffer) suitable for annealing complementary nucleic acids or for binding an antibody with a protein with which it specifically binds and one or more sample compartments. The kit of the invention may optionally comprise additional components useful for performing the methods of the invention, e.g., a sample collection vessel, e.g., a tube, and optionally, means for optimizing the amount of marker detected, for example if there may be time or adverse storage and handling conditions between the time of sampling and the time of analysis. For example, the kit can contain means for increasing the number of tumor cells in the sample, as described above, a buffering agent, a preservative, a stabilizing agent or additional reagents for preparation of cellular material or probes for use in the methods provided; and detectable label, alone or conjugated to or incorporated within the provided probe(s). In one exemplary embodiment, a kit comprising a sample collection vessel can comprise e.g., a tube comprising anti-coagulant and/or stabilizer, e.g., an RNA stabilizer, as described above, or known to those skilled in the art. The kit can further comprise components necessary for detecting the detectable label (e.g., an enzyme or a substrate). For marker sets, the kit can comprise a marker set array or chip for use in detecting the biomarkers. Kits also can include instructions for interpreting the results obtained using the kit. The kit can contain reagents for detecting one or more biomarkers, e.g., 2, 3, 4, 5, or more biomarkers described herein.

In one embodiment, the kit comprises a probe to detect at least one nucleic acid marker, e.g., a marker indicative of treatment outcome (e.g., upon NAE inhibitor treatment). In an exemplary embodiment, the kit comprises a nucleic acid probe to detect a marker nucleic acid corresponding to a marker gene described herein. The marker nucleic acid can be selected from the group consisting of SEQ ID NO: 1, 3, 5, 7, 9, 11, 13, 15, 17, 19, 21, 23, an open reading frame of SEQ ID NO: 1, 3, 5, 7, 9, 11, 13, 15, 17, 19, 21, 23, a sequence on a chromosome locus listed in Table 1, and a complement of any of the foregoing. In some embodiments, the kit comprises a probe to detect a nucleic acid corresponding to a marker gene selected from the group consisting of TET2, RUNX1, NRAS, KRAS, DNMT3A, TP53, IDH2, EZH2, IDH1, NPM1, PHF6 and ASXL1. In other embodiments, the kit comprises a probe to detect an alteration in a marker gene selected from the group consisting of TET2, RUNX1, NRAS, KRAS, DNMT3A, TP53, IDH2, EZH2, IDH1, NPM1, PHF6 and ASXL1. In an embodiment, a kit comprises probes to detect a marker set comprising two or more markers from the group consisting of TET2, RUNX1, NRAS, KRAS, DNMT3A, TP53, IDH2, EZH2, IDH1, NPM1, PHF6 and ASXL1. In another embodiment, a kit comprises a probe to detect wild type ASXL1 and a probe to detect wild type IDH1 and a probe to detect a genetic alteration, e.g., a mutation or truncation, in at least one marker gene selected from the group consisting of TET2, RUNX1, NRAS, KRAS, DNMT3A, TP53, IDH2, EZH2, NPM1 and PHF6. In an embodiment, a kit comprises a probe to detect a genetic alteration, e.g., a mutation or truncation, in a marker gene selected from the group consisting of TET2, RUNX1, NRAS, KRAS, DNMT3A and further comprises a probe to detect a genetic alteration, e.g., a mutation or truncation, in a marker gene selected from the group consisting of TP53, IDH2, EZH2, IDH1, NPM1, PHF6 and ASXL1. In related embodiments, the kit comprises a nucleic acid probe comprising or derived from (e.g., a fragment, e.g., an open reading frame, a mutant or variant (e.g., homologous or complementary) thereof) a nucleic acid sequence selected from the group consisting of SEQ ID NOs: 1, 3, 5, 7, 9, 11, 13, 15, 17, 19, 21, and 23. For kits comprising nucleic acid probes, e.g., oligonucleotide-based kits, the kit can comprise, for example: one or more nucleic acid reagents such as an oligonucleotide (labeled or non-labeled) which hybridizes to a nucleic acid sequence corresponding to a marker of the invention, optionally fixed to a substrate; and can optionally further comprise labeled oligonucleotides not bound with a substrate, a primer, a pair of PCR primers, e.g., useful for amplifying a nucleic acid molecule corresponding to a marker of the invention, molecular beacon probes, a marker set comprising oligonucleotides which hybridize to at least two nucleic acid sequences corresponding to markers of the invention, and the like. The kit can contain an RNA-stabilizing agent.

In another embodiment, a kit to assay mutational status of a protein marker of the invention can comprise one or more reagents to measure the activity of the protein. For example, the kit may comprise a reporter gene for use in the methods described herein. In another example, the kit may comprise a substrate of an enzyme, e.g., a methylation substrate of a protein marker described herein. In another example, a kit for KRAS or NRAS activity can comprise GTP precursor and reagent to measure GTPase activity.

In another embodiment, the kit comprises a probe to assay for a characteristic, e.g., size, sequence, composition or amount, of a protein marker of the invention. For kits comprising protein probes, e.g., ligand or antibody-based kits, the kit can comprise, for example: (1) a first antibody (e.g., attached to a solid support) which binds to a polypeptide corresponding to a marker of the invention, e.g., having a sequence selected from the group consisting of SEQ ID NO:2, 4, 6, 8, 10, 12, 14, 16, 18, 20, 22, and 24 or to a genetically altered form thereof, e.g., a mutation in the sequence or a truncation, e.g., as described in Table 1; and, optionally, (2) a second, different antibody which binds to either the polypeptide or the first antibody and is conjugated to a detectable label. The kit can contain a protein stabilizing agent. The kit can contain reagents to reduce the amount of non-specific binding of non-biomarker material from the sample to the probe. Examples of reagents to reduce non-specific binding include nonioinic detergents, non-specific protein containing solutions, such as those containing albumin or casein, or other substances known to those skilled in the art.

To generate antibodies for use in the methods described herein or for providing in the kits and compositions described herein, an isolated polypeptide corresponding to a marker gene of the invention, or a fragment or mutant thereof, can be used as an immunogen. For example, an immunogen typically is used to prepare antibodies by immunizing a suitable (i.e., immunocompetent) host such as a rabbit, sheep, goat, mouse, or other mammal, chicken or vertebrate. In still a further aspect, the invention provides monoclonal antibodies or antigen binding fragments thereof, which antibodies or fragments specifically bind to a polypeptide comprising an amino acid sequence selected from the group consisting of the amino acid sequences of the present invention, an amino acid sequence encoded by the cDNA of the present invention, a fragment of at least 8, 10, 12, 15, 20 or 25 consecutive amino acid residues of an amino acid sequence of the present invention, e.g., a fragment comprising the mutant amino acid sequence, or comprising a region beyond a truncation in a marker protein, an amino acid sequence which is at least 95%, 96%, 97%, 98% or 99% identical to an amino acid sequence of the present invention (wherein the percent identity is determined using the ALIGN program of the GCG software package with a PAM120 weight residue table, a gap length penalty of 12, and a gap penalty of 4) and an amino acid sequence which is encoded by a nucleic acid molecule which hybridizes to a nucleic acid molecule consisting of the nucleic acid molecules of the present invention, or a complement thereof, under conditions of hybridization of 6×SSC at 45° C. and washing in 0.2×SSC, 0.1% SDS at 65° C. The monoclonal antibodies can be human, humanized, chimeric and/or non-human antibodies. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Methods for making human antibodies are known in the art. One method for making human antibodies employs the use of transgenic animals, such as a transgenic mouse. These transgenic animals contain a substantial portion of the human antibody producing genome inserted into their own genome and the animal's own endogenous antibody production is rendered deficient in the production of antibodies. Methods for making such transgenic animals are known in the art. Such transgenic animals can be made using XENOMOUSE™ technology or by using a “minilocus” approach. Methods for making XENOMICE™ are described in U.S. Pat. Nos. 6,162,963, 6,150,584, 6,114,598 and 6,075,181, which are incorporated herein by reference. Methods for making transgenic animals using the “minilocus” approach are described in U.S. Pat. Nos. 5,545,807, 5,545,806 and 5,625,825; also see International Publication No. WO93/12227, which are each incorporated herein by reference.

Antibodies include immunoglobulin molecules and immunologically active portions of immunoglobulin molecules, i.e., molecules that contain an antigen binding site which specifically binds an antigen, such as a polypeptide of the invention or a genetically altered form thereof, e.g., a mutated amino acid or a truncation of the protein expressed by a marker gene of the invention. A molecule which specifically binds to a given polypeptide of the invention is a molecule which binds the polypeptide, but does not substantially bind other molecules in a sample, e.g., a biological sample, which naturally contains the polypeptide. Polyclonal and monoclonal antibodies can be produced by a variety of techniques, including conventional murine monoclonal antibody methodology e.g., the standard somatic cell hybridization technique of Kohler and Milstein, Nature 256: 495 (1975) the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. See generally, Harlow, E. and Lane, D. (1988) Antibodies: A Laboratory Manual, Cold Spring Harbor Laboratory Press, Cold Spring Harbor, N.Y.; and Current Protocols in Immunology, Coligan et al. ed., John Wiley & Sons, New York, 1994. Hybridoma cells producing a monoclonal antibody of the invention are detected by screening the hybridoma culture supernatants for antibodies that bind the polypeptide of interest, e.g., using a standard ELISA assay.

If desired, the antibody molecules can be harvested or isolated from the host (e.g., from the blood or serum of the host) and further purified by well-known techniques, such as protein A chromatography to obtain the IgG fraction. Alternatively, antibodies specific for a protein or polypeptide of the invention can be selected or (e.g., partially purified) or purified by, e.g., affinity chromatography to obtain substantially purified and purified antibody. By a substantially purified antibody composition is meant, in this context, that the antibody sample contains at most only 30% (by dry weight) of contaminating antibodies directed against epitopes other than those of the desired protein or polypeptide of the invention, and at most 20%, at most 10%, or at most 5% (by dry weight) of the sample is contaminating antibodies. A purified antibody composition means that at least 99% of the antibodies in the composition are directed against the desired protein or polypeptide of the invention.

An antibody directed against a polypeptide corresponding to a marker of the invention (e.g., a monoclonal antibody) can be used to detect the marker (e.g., in a cellular sample) in order to evaluate the level and pattern of expression of the marker. The antibodies can also be used diagnostically to monitor protein levels in tissues or body fluids (e.g. in a blood sample or urine) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Accordingly, in one aspect, the invention provides substantially purified antibodies or fragments thereof, and non-human antibodies or fragments thereof, which antibodies or fragments specifically bind to a polypeptide comprising an amino acid sequence encoded by a marker identified herein. The substantially purified antibodies of the invention, or fragments thereof, can be human, non-human, chimeric and/or humanized antibodies.

The invention also provides a kit containing an antibody of the invention conjugated to a detectable substance, and instructions for use. Still another aspect of the invention is a prognostic composition comprising a probe of the invention and a pharmaceutically acceptable carrier. In one embodiment, the prognostic composition contains an antibody of the invention, a detectable moiety, and a pharmaceutically acceptable carrier.

### Use of Information

In one method, information, e.g., about the mutational status of a patient's cancer, e.g., the patient's marker(s) characteristic, e.g., size, sequence, composition or amount (e.g., the result of evaluating a marker or marker set described herein), or about whether a patient is expected to have a favorable outcome, is provided (e.g., communicated, e.g., electronically communicated) to a third party, e.g., a hospital, clinic, a government entity, reimbursing party or insurance company (e.g., a life insurance company). For example, choice of medical procedure, whether to pay for a medical procedure, payment by a reimbursing party, or cost for a service or insurance can be function of the information. E.g., the third party receives the information, makes a determination based at least in part on the information, and optionally communicates the information or makes a choice of procedure, payment, level of payment, coverage. based on the information. In the method, the characteristic, e.g., size, sequence, composition or amount, of a marker or a marker set selected from or derived from Table 1 and/or described herein is determined. For example, an entity, e.g., a hospital, care giver, government entity, or an insurance company or other entity which pays for, or reimburses medical expenses, can use the result of a method described herein to determine whether a party, e.g., a party other than the subject patient, will pay for services (e.g., a particular therapy) or treatment provided to the patient. In an embodiment, the method further comprises paying for the procedure wherein the patient has a favorable outcome to therapy comprising an NAE inhibitor, e.g., pevonedistat or a pharmaceutically active salt thereof. In another embodiment, the method comprises paying for a procedure comprising treatment with pevonedistat and azacitidine.

In one embodiment, a premium for insurance (e.g., life or medical) is evaluated as a function of information about one or more marker expression levels, e.g., a marker or marker set, e.g., a level of expression associated with treatment outcome (e.g., the informative amount). For example, premiums can be increased (e.g., by a certain percentage) if the marker genes of a patient or a patient's marker set described herein have different characteristic, e.g., size, sequence, composition or amount between an insured candidate (or a candidate seeking insurance coverage) and a reference value (e.g., a non-afflicted person) or a reference sample, e.g., matched control. Premiums can also be scaled depending on the result of evaluating a marker or marker set described herein. For example, premiums can be assessed to distribute risk, e.g., as a function of marker, e.g., the result of evaluating a marker or marker set described herein. In another example, premiums are assessed as a function of actuarial data that is obtained from patients that have known treatment outcomes.

Information about marker characteristic, e.g., size, sequence, composition or amount, e.g., the result of evaluating a marker or marker set described herein, can be used, e.g., in an underwriting process for life insurance. The information can be incorporated into a profile about a subject. Other information in the profile can include, for example, date of birth, gender, marital status, banking information, credit information, children, and so forth. An insurance policy can be issued as a function of the information on marker characteristic, e.g., size, sequence, composition or amount, e.g., the result of evaluating a marker or marker set described herein, along with one or more other items of information in the profile. For example, a first entity, e.g., an insurance company, can use the outcome of a method described herein to determine whether to continue, discontinue, enroll an individual in an insurance plan or program, e.g., a health insurance or life insurance plan or program.

In one aspect, the disclosure features a method of providing data. The method includes providing data described herein, e.g., generated by a method described herein, to provide a record, e.g., a record described herein, to proceed a payment. In some embodiments, the data are provided by computer, compact disc, telephone, facsimile, email, or letter. In some embodiments, the data are provided by a first party to a second party. In some embodiments, the first party is selected from a healthcare provider, a treating physician, a health maintenance organization (HMO), a hospital, a governmental entity, or an entity which sells or supplies the drug. In some embodiments, the second party is a third party payor, an insurance company, employer, employer sponsored health plan, HMO, or governmental entity. In some embodiments, the first party is selected from a healthcare provider, a treating physician, an HMO, a hospital, an insurance company, or an entity which sells or supplies the drug and the second party is a governmental entity. In some embodiments, the first party is selected from a healthcare provider, a treating physician, an HMO, a hospital, an insurance company, or an entity which sells or supplies the drug and the second party is an insurance company.

In another aspect, the disclosure features a record (e.g., computer readable record) which includes a list and value of characteristic, e.g., size, sequence, composition or amount for the marker or marker set for a patient. In some embodiments, the record includes more than one value for each marker.

The present invention will now be illustrated by the following Examples, which are not intended to be limiting in any way.

### EXAMPLES

### Example 1. Phase 1b Clinical Study of Pevonedistat and Azacitidine

The clinical trial (NCT01814826) pevonedistat (PEV) with azacitidine (AZA) was based on synergistic activity seen preclinically. Primary objectives included safety and tolerability, and secondary objectives included pharmacokinetics (PK) and disease response. Patients ≥60 years with treatment-naïve AML, unfit for standard induction therapy. A report is published as Swords et al. (2018) Blood 131:1415-1424.

Introduction

Current therapy in acute myeloid leukemia (AML) is inadequate.¹⁻⁵ Although some progress has been made in this disease, the prognosis for older patients, deemed unfit to receive intensive chemotherapy, remains very poor.^{4,5} The use of hypomethylating agents (HMAs) as alternative induction therapies for these patients has become commonplace. Two large randomized studies reported higher rates of remission for older patients treated with 5-azacitidine (AZA) compared with conventional care approaches, which included supportive care.^{6,7} Considering the widespread use of AZA in older patients who are not candidates for chemotherapy, combination studies with promising new agents are actively enrolling.⁸.

We previously reported the therapeutic potential of single-agent pevonedistat (PEV) (previously TAK-924/MLN4924) in patients with AML.⁹ PEV is a small molecule inhibitor of the NEDD8 activating enzyme (NAE), which processes Neural Cell Developmentally-Downregulated 8 (NEDD8) for binding to target substrates.¹⁰⁻¹² The best characterized NAE targets in cells are the Cullin-RING E3 ubiquitin ligases (CRLs), which direct the degradation of specific substrates (e.g., p27, CDT1, Nrf-2) through the proteasome.¹³⁻¹⁷ In response to PEV treatment, impaired NAE activity leads to CRL substrate accumulation, causing anti-proliferative effects in AML.¹⁸ A variety of mechanisms are implicated in driving these effects, including disruption of cellular redox via stabilization of pIKB (a critical mediator of cell killing),¹⁸ DNA replication, and cell cycle arrest.¹⁹ In a phase 1b study of patients with relapsed/refractory AML and MDS (myelodysplastic syndrome), PEV was administered as a 1-hour IV infusion on days 1, 3, and 5 (schedule A, n=27) or days 1, 4, 8, and 11 (schedule B, n=26) every 21 days.⁹ The maximum tolerated doses (MTDs) for schedules A and B were 59 and 83 mg/m², respectively. On schedule A, elevation of alanine aminotransferase (ALT)/aspartate aminotransferase (AST) was dose limiting. Multi-organ failure (MOF) was dose limiting on schedule B. Overall response rate (ORR) in patients treated at or below the MTD was 17% (4/23, 2 complete remissions [CRs], 2 partial remissions [PRs]) for schedule A and 10% (2/19, 2 PRs) for schedule B.⁹

To identify clinically effective PEV combinations, a high-throughput viability screen in AML cells confirmed that combined treatment using PEV with either decitabine or AZA was synergistically lethal by Combination Index and Blending Synergy Analysis.¹⁹ In the case of AZA, combined treatment with PEV significantly increased DNA damage and cell death when compared with either agent alone, as measured by immunoblotting and flow cytometry analysis of cell cycle distributions. In vivo studies were performed in AZA-resistant HL-60 and THP-1 xenografts. Although the doses of PEV and AZA would be sub-therapeutic if used as single-agent treatment, the combination led to complete and sustained tumor regression in these models.¹⁹ The mechanisms underlying the observed synergistic effects are currently under investigation.¹⁹ Considering the promising clinical data for PEV as a single agent and its enhanced antitumor activity when combined with AZA in laboratory models, we conducted a phase 1b trial of PEV combined with AZA for older patients with AML, deemed unfit to receive intensive chemotherapy.

### Materials and Methods

Patients Eligible patients were ≥60 years old with untreated AML, considered unlikely to benefit from standard induction defined by ≥1 of the following: age ≥75 years, presence of antecedent MDS, adverse cytogenetic risk, and Eastern Cooperative Oncology Group performance status (ECOG PS) of 2. Other inclusion criteria included ECOG PS of 0-2; adequate renal function (calculated creatinine clearance >50 mL/min), adequate hepatic function (bilirubin within normal range, AST and ALT≤2.5× upper limit of normal [ULN]), and adequate cardiac function (B-type natriuretic peptide ≤1.5×ULN, left ventricular ejection fraction ≥50% and pulmonary artery systolic pressure ≤1.5×ULN). Exclusion criteria included treatment with an investigational anti-leukemic agent within 14 days prior to entering study, uncontrolled inter-current illness, and known infection with HIV and/or viral hepatitis B or C. Moderate and strong CYP3A inhibitors or chronic continuous use of CYP3A inducers were not permitted. All procedures performed in studies involving human participants were in accordance with the ethical standards of the institutional and/or national research committee and with the 1964 Declaration of Helsinki and its later amendments or comparable ethical standards.

Study Design

This open-label, phase 1b study was conducted at 10 sites across the United States. The primary objectives were to determine the dose-limiting toxicities (DLTs) and MTDs of PEV when combined with fixed doses of AZA. Secondary objectives included descriptions of PEV pharmacokinetics (PK) in whole blood and a preliminary assessment of anti-tumor activity. PEV was administered via a 60-minute intravenous (IV) infusion on days 1, 3, and 5 in escalating doses beginning at 20 mg/m². AZA was administered IV only during dose escalation and IV or subcutaneously (SC) during dose expansion, in standard doses (75 mg/m²) on days 1-5, 8, and 9. Cycles were repeated every 28 days, and treatment continued until disease progression or unacceptable toxicity. Dose escalation was performed using continual reassessment method (CRM), which used non-informative beta priors with a target DLT rate of 25%. The MTD was determined to be the highest dose level at which at least six patients were dosed (at any dose level) and the CRM algorithm did not recommend escalation or de-escalation. DLTs were defined in cycle 1 only as grade ≥3 toxicity related to study drug (exceptions were arthralgia/myalgia despite optimal use of analgesia, fatigue <1 week, hypophosphatemia, and prolonged PT/aPTT without clinical bleeding). Upon determining the MTD, there was a pre-planned expansion of 55 patients at the MTD to better define the safety profile and to gather a preliminary assessment of efficacy at the recommended phase 2 dose (RP2D).

Safety and Efficacy Assessments

Patient demographics and medical history were recorded at baseline. Adverse event (AE) assessments, physical examination, vital signs, and ECOG PS were documented at baseline and on day 1 of subsequent cycles for the duration of the study. Safety was assessed from informed consent to 30 days after final doses of study therapy. Treatment-emergent (all-cause) AEs were graded according to the National Cancer Institute's Common Terminology Criteria for AEs, version 4.03.²⁰ Patients with AML were assessed for efficacy according to published International Working Group criteria.²¹

PK Analysis

Serial blood samples for the determination of PEV concentrations were obtained during the first cycle of treatment, at pre-specified time points before and up to 48 hours after the start of the infusion on days 1 and 5. Non-compartmental analyses (using WinNonlin software, Version 6.2, Pharsight Corporation, Cary, N.C.) were used to estimate the observed maximum concentration (Cₘₐₓ; theoretical end-of-infusion concentration), the time at which Cₘₐₓ occurred (Tₘₐₓ), the area under the plasma concentration-time curve from time 0 to 24 hours post-dose (AUC₂₄ₕᵣ), the area under the plasma concentration-time curve from time 0 to the end of the dosing interval (AUC₀₋tau), and data permitting, the terminal disposition phase half-life (t_{1/2}).

Statistical Analysis

Response rates were reported along with their 95% confidence intervals (CI) using a 2-sided exact binomial test. Overall survival and 1-year survival rates along with their 2-sided 95% CI were estimated by using Kaplan-Meier methods.

Next-Generation Sequencing

High-quality DNA extracted from either bone marrow aspirates and/or blood, together with matched buccal swab samples was available for 28 of 52 response-evaluable patients at time of screening. A targeted next-generation sequencing (NGS) panel consisting of 116 genes, comprising genes implicated in myeloid neoplasms as well as genes involved in pathways modulated by PEV (Table 2) was constructed. Samples were sequenced on an Illumina HiSeq with 76 bp, paired end reads to meet a mean target coverage of 500×(±5%) (tumor average [avg] coverage=10,430×, normal avg coverage=10,296×) as measured by the Broad's Picard bioinformatics pipeline. De-multiplexed, aggregated Picard BAM files were analyzed to identify single nucleotide variants (SNVs) and insertions/deletions (indels). SNV and indels are identified with VarScan.v2.3.9 and false positives are filtered with the fpfilter function. After false positive removal, alterations in highly mutated AML genes are kept if they match the table of known variations (Table 3). Genes with low AML mutation frequency were kept if the P value was less than 0.01 and the coverage was 100× or greater. Notably, this targeted NGS methodology did not allow for the identification of mutations in the CEBPa gene and FLT3-ITD mutations.

TABLE 2
List of 116 genes included in the targeted NGS panel

| | | | | | | | |
|---|---|---|---|---|---|---|---|
| PHF6 | CARD11 | FGFR2 | JAK2 | NRAS | SOCS1 | CIITA | P2RY8 |
| MILL | CBL | FGFR3 | JAK3 | PAX5 | TET2 | ETV6 | PASD1 |
| APC | CDKN2A | FLT3 | KDM6A | PDGFRA | TNFAIP3 | GNA13 | PCLO |
| EGFR | CEBPA | GATA1 | KIT | PIK3CA | TP53 | HIST1H1C | PIM1 |
| MET | CREBBP | GATA2 | KRAS | PRDM1 | WT1 | HIST1H3B | POU2F2 |
| STK11 | CRLF2 | GNAS | MPL | PTEN | ACTB | HLA-A | SOCS1 |
| ABL1 | CSF1R | HRAS | MT-ND4 | PTPN11 | B2M | KRTAP5-5 | STAT3 |
| AKT1 | CTNNB1 | IDH1 | MYD88 | RB1 | BTG1 | LOC153328 | SYK |
| ASXL1 | DNMT3A | IDH2 | NF1 | RUNX1 | CCND3 | MEF2B | SYN2 |
| ATM | EP300 | IKZF1 | NOTCH1 | SF3B1 | CD58 | MLL2 | TMSL3 |
| ATRX | EZH2 | IL7R | NOTCH2 | SMAD4 | CD70 | NFKBIA | TNFRSF14 |
| BRAF | FBXW7 | JAK1 | NPM1 | SMARCB1 | CD79B | OR6K3 | UBE2A |
| UNC5D | PSMB10 | ERN1 | PSMB8 | TRAF2 | AKT2 | XBP1 | NFKB2 |
| XPO1 | PSMB5 | MAP3K7 | PSMB9 | TRAF5 | FGFR1 | IKBa | PSMB1 |
| | | (TAK1) | | | | | |
| CCND1 | PSMB6 | TNFRSF11A | TRAF3 | | | | |
| | | (RANK) | | | | | |

TABLE 3
List of 38 frequently altered genes with mutations
identified using the targeted NGS panel

| | | Truncating |
|---|---|---|
| Gene | Amino acid ranges* | ranges* |
| ASXL1 | None | 327-1540 |
| DNMT3A | 290-374; 626-910 | All |
| EZH2 | 1-340; 428-476; 502-611; 617-738; | All |
| GATA1 | ExACFreq < 0.01 | All |
| GNAS | 844-844; 201-201 | None |
| IDH1 | 126-138 | None |
| IDH2 | 134-146; 164-180 | None |
| IKZF1 | 142-196 | All |
| JAK2 | 505-547; 617-617; 683-683; 867-867; 873- | None |
| | 873; 933-933 | |
| KRAS | ExACFreq < 0.01 | None |
| MPL | 500-520 | None |
| NOTCH1 | 1530-1795; | 2061-2555 |
| NPM1 | None | All |
| NRAS | ExACFreq < 0.01 | None |
| PHF6 | 197-353; | All |
| RUNX1 | ExACFreq < 0.01 | All |
| SF3B1 | 600-780; | None |
| TET2 | 1104-1481; 1843-2002; | All |
| TP53 | ExACFreq < 0.01 | All |

*The accepted amino acid changes and truncating ranges used for mutation calling from NGS sequence data in frequently-mutated genes. ‘None’ indicates that no amino acid changes or truncating mutations are accepted. ‘ExAcFreq < 0.01’ indicates that all mutations are accepted if the allele is present in less than 1% of the ExAC population. ‘All’ indicates that all of the truncating mutations are accepted.

Results

Sixty-four patients were enrolled into two dose levels in this study and included in all assessments of safety, demographics, and baseline disease characteristics. Efficacy assessments were confined to the MTD cohort patients (PEV 20 mg/m²+AZA, [n=61]), treated at the RP2D for all subsequent phase 2 and 3 studies.

Patient Characteristics

The patient demographics are displayed in Table 4. Sixty-four patients with a median age of 75 years (range 61-89) were treated on the study. Of these, 53% were male. Most patients (78%) had an ECOG PS of 0-1. Over half of the patients enrolled had de novo AML (56%). Median marrow blast percentage was 38.5% (range 5-92), 50% had intermediate-risk, 28% had adverse-risk, and 3% had favorable-risk cytogenetics.

TABLE 4
Baseline patient demographics

| | ITT cohort (n = 64) | |
|---|---|---|
| Characteristics* | | |
| Median age, years (range) | 75 | (61-89) |
| Male, n (%) | 34 | (53) |
| White, n (%) | 58 | (91) |
| ECOG PS, n (%) | | |
| 0 | 27 | (42) |
| 1 | 23 | (36) |
| 2 | 14 | (22) |
| Primary diagnosis | | |
| De novo AML | 36 | (56) |
| Secondary AML | 28 | (44) |
| Median marrow blasts, % (range) | 38.5 | (5-92) |
| Cytogenetics, n (%)† | | |
| Adverse | 18 | (28) |
| Intermediate | 32 | (50) |
| Favorable | 2 | (3) |
| Unclassified | 9 | (14) |
| Not available | 3 | (5) |

CALGB, Cancer and Leukemia Group B; ITT, intent-to-treat.
*Data cutoff: September 2016.
†Cytogenetic risk centrally assessed and reported according to CALGB criteria

DLTs and MTD

determination PEV dosing was started at 20 mg/m² (n=6) and increased to 30 mg/m² (n=3) in the absence of DLTs. At the 30 mg/m² dose level, two of the three patients experienced a DLT: 1 patient had persistent grade 2 bilirubin elevation and 1 patient had reversible grade 4 AST elevation. Transaminase and bilirubin elevations were transient and clinically inconsequential in both patients (resolving to grade 1 or baseline levels within 1 week of withdrawal from study) (Table 5). The MTD for PEV was declared at 20 mg/m² when combined with AZA in standard doses, based on the final posterior estimate of probability of toxicity at 20 mg/m² being 24% using the CRM model. In the MTD expansion cohort (n=55), two additional patients experienced DLTs (grade ≥3 transaminase elevation) and were successfully re-challenged with a reduced dose of PEV. Both patients remained on study without further hepatic toxicity.

TABLE 5
Overall response rates of the ITT patient population

| | Response rate in ITT cohort, % (95% CI) | | | | | | | |
|---|---|---|---|---|---|---|---|---|
| | ORR | | CR | | CRi | | PR | |
| Total patients (n = 64)* | 50 | (37-63) | 31 | (20-44) | 8 | (3-17) | 11 | (5-21) |
| AML subtype | | | | | | | | |
| De novo AML (n = 36) | 53 | (35-70) | 33 | (19-51) | 8 | (2-22) | 11 | (3-26) |
| Secondary AML (n = 28) | 46 | (28-66) | 29 | (13-49) | 7 | (1-24) | 11 | (2-28) |
| Bone marrow blast count | | | | | | | | |
| <30% (n = 25) | 52 | (31-72) | 28 | (12-49) | 12 | (3-31) | 12 | (3-31) |
| ≥30% (n = 39) | 49 | (32-65) | 33 | (19-50) | 5 | (1-17) | 10 | (3-24) |
| Cytogenetic risk | | | | | | | | |
| Intermediate (n = 32) | 44 | (26-62) | 28 | (14-47) | 3 | (0-16) | 13 | (4-29) |
| Adverse (n = 18) | 44 | (22-69) | 28 | (10-53) | 11 | (1-35) | 6 | (0-27) |
| AZA + PEV exposure | | | | | | | | |
| <6 cycles (n = 41) | 32 | (18-48) | 15 | (6-29) | 7 | (2-20) | 10 | (3-23) |
| ≥6 cycles (n = 23) | 83 | (61-95) | 61 | (39-80) | 9 | (1-28) | 13 | (3-34) |

*Considering the 3 patients who were treated at the 30 mg/m² PEV dose: 1 patient discontinued following a best response of SD which lasted ~1 month, and the patient discontinued due to a SAE of grade 3 pneumonia; 1 patient achieved a CR lasting ~4 months, at which point this patient discontinued treatment due to progressive disease; and 1 patient had a DLT of grade 4 AST/ALT elevation on Day 1, further dosing were held on Day 3 and Day 5, patient discounted study due to symptomatic deterioration on Day 8 prior to first post-baseline assessment.

Safety

Treatment-emergent adverse event data for the intention-to-treat cohort patients (n=64) are summarized: Patients received a median of 4 cycles (range 1-37), and 23/64 patients (36%) received ≥6 cycles of therapy. The most common AEs were constipation (48%), nausea (42%), fatigue (42%), and anemia (39%). Fifty-three patients (83%) experienced grade ≥3 AEs; the most frequent (≥15%) were anemia, febrile neutropenia (each 30%), thrombocytopenia (23%), neutropenia (20%), and pneumonia (17%). Increased liver enzymes (grade ≥3 increase in either AST or ALT) were reported in 6% of patients. Forty-four patients (69%) experienced serious AEs; the most frequent (≥10%) were febrile neutropenia (25%) and pneumonia (14%). In addition to the 2 patients who withdrew due to DLTs, two additional patients withdrew from the study due to febrile neutropenia, considered by the investigator to be related to both PEV and AZA. There were 11 on-study deaths due to progression of disease or disease-related events, unrelated to study therapy.

PK

All relevant PK parameters of PEV administered in combination with AZA are summarized in Table 6. Mean and individual PK profiles of PEV on cycle 1, day 1 and day 5 exhibited a biphasic disposition phase, whereby PEV plasma concentrations were measurable up to 24 hours post-dose in all patients and up to 48 hours post-dose in approximately half of the patients. Systemic exposure data indicate that PEV PK was not altered in the presence of AZA when compared with historical single-agent data.^{9,22} Additionally, when comparing individual PK profiles on day 5 versus day 1, PEV exposures remained unchanged following five days of continuous dosing with AZA (observed accumulation ratio R_{ac}˜1).

TABLE 6
Summary of plasma PK parameters of PEV in combination with IV/SC
AZA

| | |
|---|---|
| 20 mg/m² cohort | 30 mg/m² cohort |

| Dose escalation* | IV cohort (N = 6) | SC cohort (N = 3) |
|---|---|---|
| Cycle 1, day 1 | | |

| | | | | |
|---|---|---|---|---|
| Tₘₐₓ (h) | 1.06 | (0.97-2.27) | 0.98 | (0.97-1.00) |
| Cₘₐₓ (ng/mL) | 158 | (51.4) | 299 | (29.9) |
| AUC₂₄ (ng/h/mL) | 990 | (28.0) | 1640 | (26.4) |
| AUC₄₈ (ng/h/mL) | 1110 | (30.6) | 1770 | (25.8) |
| T_{1/2} (h) | 7.80 | (1.13) | 7.39 | (0.699) |

| | | | |
|---|---|---|---|
| Cycle 1, day 5 | | | |
| Tₘₐₓ (h) | 0.99 | (0.97-1.48) | —† |
| Cₘₐₓ (ng/mL) | 165 | (48.4) | — |
| AUC₂₄ (ng/h/mL) | 986 | (38.4) | — |
| AUC₄₈ (ng/h/mL) | 1090 | (35.6) | — |
| T_{1/2} (h) | 7.98 | (0.818) | — |

| | | |
|---|---|---|
| MTD expansion | IV cohort (N = 26) | SC cohort (N = 28)‡ |

| | | | | |
|---|---|---|---|---|
| Cycle 1, day 1 | | | | |
| Tₘₐₓ (h) | 1.01 | (0.65-2.03) | 1.00 | (0.88-3.00) |
| Cₘₐₓ (ng/mL) | 155 | (41.2) | 152 | (32.3) |
| AUC₂₄ (ng/h/mL) | 861 | (26.7) | 890 | (29.3) |
| AUC₄₈ (ng/h/mL) | 976 | (24.6)§ | 1000 | (23.7)\|\| |
| T_{1/2} (h) | 7.45 | (1.85) | 7.30 | (1.76) |
| Cycle 1, day 5 | | | | |
| Tₘₐₓ (h) | 1.00 | (0.92-2.00) | 0.98 | (0.83-2.00) |
| Cₘₐₓ (ng/mL) | 164 | (41.3) | 148 | (40.6) |
| AUC₂₄ (ng/h/mL) | 921 | (23.8) | 926 | (25.5) |
| AUC₄₈ (ng/h/mL) | 1100 | (22.5)¶ | 1100 | (21.4)# |
| T_{1/2} (h) | 8.07 | (2.14) | 7.89 | (1.76) |

Parameters are presented as geometric mean (CV %) unless specified otherwise; Tₘₐₓ (median, range); t_{1/2} (mean, SD).
*All patients on the dose escalation cohorts received IV PEV (20 mg/m² cohort, N = 6; 30 mg/m² cohort, N = 3).
†NR: only 1 patient was evaluable on cycle 1, day 5 as dosing was halted due to AEs.
‡One patient is not PK-evaluable due to insufficient concentration-time data collected during cycle 1 for analysis.
§N = 12.
||N = 11.
¶N = 13.
#N = 18.

Efficacy

Considering the 3 patients who were treated at the 30 mg/m² PEV dose, 1 patient discontinued following a best response of SD which lasted ˜1 month, and discontinued due to an SAE (grade 3 pneumonia); 1 patient achieved a CR which had a duration of ˜4 months, at which point the patient discontinued treatment due to progressive disease; and 1 patient discontinued from study prior to the first disease assessment due to symptomatic deterioration (Table 5).

Among the 61 patients in the MTD cohort, 9 had no post-baseline disease assessments and were therefore deemed not evaluable for response. One patient withdrew consent, 1 was lost to follow-up, and 7 discontinued treatment prior to their first marrow assessment due to experiencing SAEs: 3 patients had pneumonia, and 1 patient each had sepsis, mental health status change, pulmonary edema/congestive heart failure, or multiple organ failure. The ORR in the 52 response-evaluable patients was 60% (19 CR, 5 CR with incomplete hematologic recovery [CRi], 7 PR; Table 7), with a median duration of remission of 8.3 months (95% CI, 5.52-12.06 months; response rates within the ITT population are noted in Table 5). Of the responding patients, 61% (19/31) responded within the first two cycles of treatment, 14 had responses lasting ≥4 cycles, and 2 proceeded to allogeneic stem cell transplantation. In total, 3 patients proceeded to stem cell transplantation, as they met physiologic requirements and agreed to pursue the treatment. The ORR was 59% (13/22; 7 CR, 3 Cri, 3 PR) versus 60% (18/30; 12 CR, 2 Cri, 4 PR) for patients with low- (<30%) versus high- (≥30%) marrow blast percentage; 58% (18/31; 11 CR, 3 CRi, 4 PR) versus 62% (13/21; 8 CR, 2 CRi, 3 PR) for de novo versus secondary AML patients; 57% (13/23; 8 CR, 1 CRi, 4 PR) versus 50% (8/16; 5 CR, 2 CRi, 1 PR) for intermediate-risk versus adverse-risk patients. As expected, patients were more likely to respond if they received ≥6 cycles versus <6 cycles of treatment (ORR 83% [19/23]; 14 CR, 2 CRi, 3 PR) versus 41% (12/29; 5 CR, 3 CRi, 4 PR]). We further scrutinized timing of the responses achieved between the patients, and observed that majority of the patients achieved responses within the first 2-4 cycles regardless of how long they have been treated. Similar results across all categories were obtained when the response data were analyzed based on the ITT population as listed in Table 5. Fourteen patients (half with adverse cytogenetics) remained on the study for more than 1 year (13 cycles, maximum 48+ cycles, 2 still active on study at the time of writing this manuscript) in whom the best responses were CR/CRi (n=11), PR (n=2), or stable disease (SD; n=1). Among the entire cohort of 61 patients treated at the MTD (median follow-up of 21.2 months), survival at 6 months was 52% (95% CI, 38%-63%) and 45% at 1 year (95% CI, 32%-57%); median overall survival (OS) was 7 months (95% CI, 4.5-14.5 months), and 11.2 months (95% CI, 3.5-25.3 months) versus 5.6 months (95% CI, 4.3-14.4 months) for secondary AML versus de novo patients. We evaluated the overall survival differences between the patients who had achieved CR, CRi/PR, and no CR/CRi/PR and observed statistically significant differences (log-rank p-value<0.05) between the Kaplan-Meier survival curves (CR versus CRi/PR groups showed median OS of 18.8 months versus 8.3 months, respectively). The median overall survival was 11.2 months (95% CI, 4.5 months-NE) versus 5.2 months (95% CI, 3.5-14.4 months) for patients with low (<30%) versus high (≥30%) marrow blasts; and 16.1 months (95% CI, 3.6-25.3 months) versus 5.3 months (95% CI, 4.3-12.8 months) for patients aged 65-74 versus ≥75 years, respectively.

TABLE 7
Overall response rates

| | Response rate in MTD cohort, % (95% CI) | | | | | | | |
|---|---|---|---|---|---|---|---|---|
| | ORR | | CR | | CRi | | PR | |
| Evaluable patients (n = 52) | 60 | (45-73) | 37 | (24-51) | 10 | (3-21) | 13 | (6-26) |
| AML subtype | | | | | | | | |
| De novo AML (n = 31) | 58 | (39-76) | 35 | (19-55) | 10 | (2-26) | 13 | (4-30) |
| Secondary AML (n = 21) | 62 | (38-82) | 38 | (18-62) | 10 | (1-30) | 14 | (3-36) |
| Bone marrow blast count | | | | | | | | |
| <30% (n = 22) | 59 | (36-79) | 32 | (14-55) | 14 | (3-35) | 14 | (3-35) |
| ≥30% (n = 30) | 60 | (41-77) | 40 | (23-59) | 7 | (1-22) | 13 | (4-31) |
| Cytogenetic risk | | | | | | | | |
| Intermediate (n = 23) | 57 | (35-77) | 35 | (16-57) | 4 | (0-22) | 17 | (5-39) |
| Adverse (n = 16) | 50 | (25-75) | 31 | (11-59) | 13 | (2-38) | 6 | (0-30) |
| AZA + PEV exposure | | | | | | | | |
| <6 cycles (n = 29) | 41 | (24-61) | 17 | (6-36) | 10 | (2-27) | 14 | (4-32) |
| ≥6 cycles (n = 23) | 83 | (61-95) | 61 | (39-80) | 9 | (1-28) | 13 | (3-34) |

Molecular Analysis

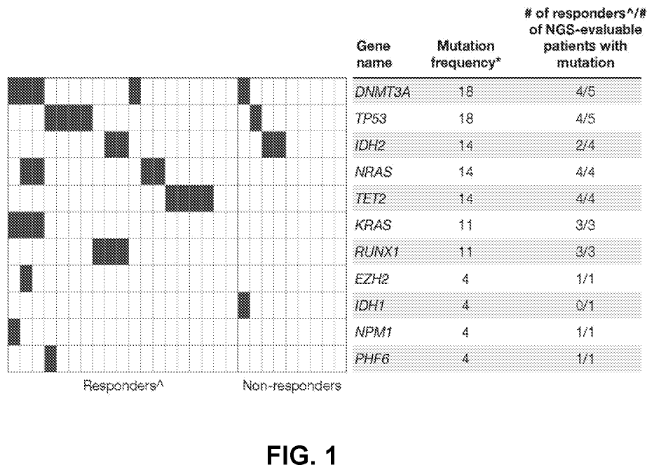

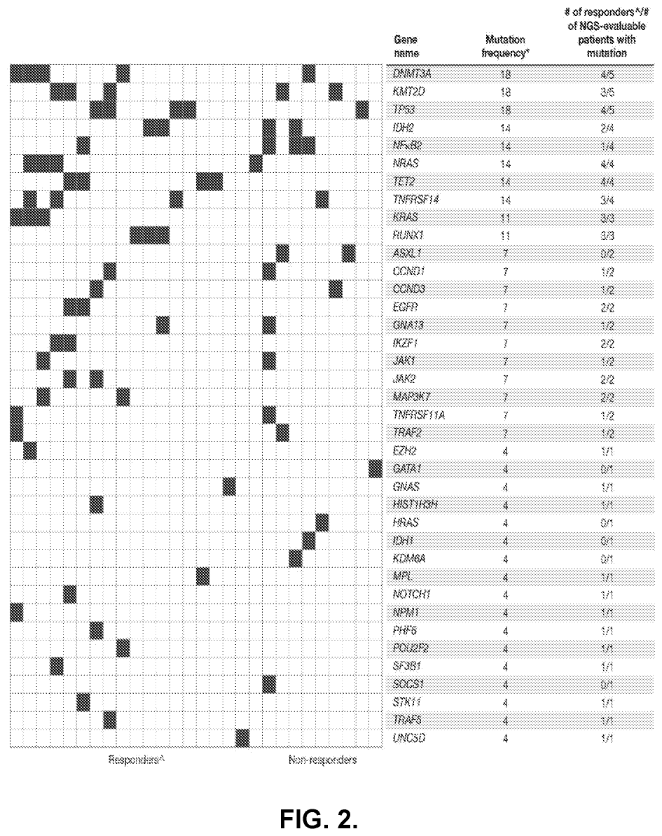

Targeted NGS identified a heterogenous mutation profile for the tumor DNA samples sequenced. Only 38 of the 116 genes sequenced were shown to be mutated in this patient cohort with 1-7 genes mutated per patient. FIG. 1 provides a summary of baseline mutations correlated with response for 11 frequently mutated AML genes. In this subset analysis, the mutational frequency of these genes, except for TP53 (5/28 patients; 18%), were consistent with frequencies previously published.²³ Mutation status and frequencies for remaining genes is provided in FIG. 2.

Discussion

Optimal management of newly diagnosed ANIL patients, unfit for induction therapy is a topic of considerable debate. Guideline recommendations for the treatment of this group include the use of HMAs (AZA or decitabine).²⁴ AZA was shown to prolong OS compared with conventional care regimens (CCRs) in the subset of older patients with 20%-30% bone marrow blasts on the phase 3 AZA-001 trial.⁷ Similarly, AZA was associated with a median OS of ˜10 months in patients with AML who participated on the Austrian AZA registry.²⁵ The phase 3 AZA-AML-001 study⁶ prospectively randomized older unfit patients with increased marrow blasts (>30%) to receive AZA or CCR (physician's choice of best supportive care only, low-dose cytarabine, or standard induction chemotherapy). In this trial, response rates for AZA versus CCR were 27.8% versus 25.1%, median OS was 10.4 versus 6.5 months, and 1-year survival was 46.5 versus 34.2%. In an attempt to improve on these data, a number of early phase clinical trials have tested the potential of newer agents when combined with AZA.^{8,26-28} Of these, PEV, a novel inhibitor of NAE, potently impairs the viability of cancer cells in laboratory models of AML¹⁸ as well as other tumor types.^{12,29-42} Several pre-clinical PEV combination approaches have now been published (including PEV/AZA combinations in AML).^{19,30,43-59} Safety and efficacy data are available on over 300 patients treated with PEV from early phase studies in both solid and hematologic malignancies including AML.⁹ Here, we tested for the first time the potential of PEV to enhance the activity of AZA in patients with AML considered unfit for intensive chemotherapy.

Overall, the combination of PEV and AZA in this older population was well tolerated. The nature and frequency of the toxicities typically observed for AZA monotherapy (fatigue, gastrointestinal toxicity, myelosuppression, and SC injection site pain)^{6,7} did not change significantly with the addition of PEV in this study. Transient elevation in liver enzymes was dose limiting for 4 patients, none of whom experienced clinical sequelae. Two of these patients were successfully re-challenged with lower doses of PEV and remained on protocol. PEV-related hepatic toxicity has been reported in other studies.^{36-38,60} In the dose escalation phase of this study, we utilized a more conservative lower starting dose of 20 mg/m² of pevonedistat compared with doses used in previous single-agent phase 1 studies, primarily to ensure safety. Azacitidine is metabolized in the liver, and hepatoxicity with single-agent AZA has been noted in animal studies and in patients,⁶¹ and elevated LFTs had previously been determined to be dose limiting in single-agent pevonedistat phase 1 studies.⁹ Proposed on-target mechanisms accounting for this toxicity include the disruption of cytoskeletal proteins in hepatic cells as well as sensitization of these cells to toxic cytokines, such as TNF-α.⁶⁰ The main reasons for withdrawal from study were disease progression and AEs (mainly unrelated to study therapy). Four patients (6.2%) came off protocol for therapy-related events (2 of these patients had DLTs, the other 2 had febrile neutropenia considered by investigators to be therapy-related). Eleven patients (17.1%) died from disease progression, and no toxic deaths were reported.

The median number of cycles of treatment with AZA plus PEV was 4, and most patients received 6 or more cycles of therapy. This is noteworthy as the addition of new agents to AZA can increase toxicity and potentially compromise total AZA exposure if patients are withdrawn early. In a recent placebo-controlled randomized study (n=102), the oral histone deacetylase inhibitor pracinostat⁸ failed to increase the efficacy of AZA alone in patients with high-risk MDS. This was attributed to higher rates of early discontinuation due to AEs (within the first two cycles) for patients treated with combined therapy. In the North American Intergroup MDS study⁶² comparing AZA plus lenalidomide versus AZA plus vorinostat (a histone deacetylase inhibitor) versus AZA alone (n=277), patients randomized to combination treatment were more likely to discontinue therapy early, more likely to undergo off-protocol dose modification, and less likely to undergo subsequent bone marrow biopsies to assess response. This study also failed to demonstrate an advantage for AZA combinations over AZA alone in a similar patient population.

Overall responses reported for AZA and PEV in this study compare favorably to AZA monotherapy in population matched controls. In the large randomized French study comparing AZA monotherapy to conventional care, patients randomized to AZA alone achieved a composite CR/PR rate of 29%,⁶ compared to 60% on the current study. The characteristics of the responses observed in this trial suggest benefit from the addition of PEV. Most responding patients achieved their responses within two cycles of therapy (61%), and almost all the responses reported occurred within four cycles of therapy (90%). Notably, bone marrow blast percentage or cytogenetic risk did not appear to influence the likelihood of achieving response following treatment with PEV and AZA in this study. For patients receiving <6 cycles (n=29) of therapy, ORR was 41%; for those who received ≥6 cycles (n=23) of therapy, ORR was 83%. These responses are explained, in part, by the favorable non-overlapping toxicity profile of PEV, which allowed for optimal AZA dosing in this study. With a median follow up of 21.2 months, median OS was 7 months (6-month OS was 52%, 1-year OS was 45%). Several patients continue on PEV and AZA at time of publication. 95% CI, 35-97%) patients with TP53-mutated AML achieved CR/CRi/PR, and four of six remained on study for >10 cycles. The mutational frequency of TP53 on this study (8/52 [15.4%]) was comparatively higher than that previously reported (15.4% versus 7%),⁶³ perhaps suggesting a more aggressive disease in patient population enrolled in this study, as TP53 alterations in AML are generally associated with older age, genomic complexity, monosomal karyotype, and shorter OS.⁶³ Nonetheless, responses were seen in patients who often have refractory disease. It is worth noting that in a recent prospective study of ANIL patients, patients with TP53-mutated ANIL responded well to extended-dose decitabine, implying a potential advantage to strategies that include azanucleosides in TP53-mutated AML.⁶⁴ Beyond TP53 mutations, NGS performed on a subset of treated patients (FIG. 4) did not reveal a robust biomarker of response to PEV but did confirm responses independent of mutational profile.

The development of PEV as a new therapeutic strategy for patients with myeloid neoplasms continues to expand. Rational combination studies informed byp re-clinical studies are being planned with both standard (PEV plus decitabine, PEV plus low dose cytarabine) and investigational agents (eg, Bel 2 inhibitors and others) in AML, MDS, and in ‘overlap syndromes’ (myelodysplastic syndromes/myeloproliferative neoplasms—MDS/MPNs). Further studies will be guided by a deeper understanding of responsiveness to therapy with PEV. For example, given that PEV can repress NF-kB dependent gene expression, it has been proposed PEV could modulate overexpression of the NF-kB dependent micro-RNA MIR155HG, which may offer an advantage to patients with normal karyotype AML, where this micro-RNA adversely impacts survival.⁴⁷ In summary, this phase 1b trial for older patients with AML unfit for high-dose induction therapy, combined treatment with PEV and AZA was well tolerated. The timing and frequency of responses suggest benefit from the addition of PEV compared to AZA alone.

### REFERENCES

1. Burnett A K, Milligan D, Goldstone A, et al. The impact of dose escalation and resistance modulation in older patients with acute myeloid leukaemia and high risk myelodysplastic syndrome: the results of the LRF AML14 trial. Br JHaematol. 2009; 145(3):318-332.
2. Burnett A K, Milligan D, Prentice A G, et al. A comparison of low-dose cytarabine and hydroxyurea with or without all-trans retinoic acid for acute myeloid leukemia and high-risk myelodysplastic syndrome in patients not considered fit for intensive treatment. Cancer. 2007; 109(6):1114-1124.
3. Burnett A K, Russell N H, Kell J, et al. European development of clofarabine as treatment for older patients with acute myeloid leukemia considered unsuitable for intensive chemotherapy. J Clin Oncol. 2010; 28(14):2389-2395.
4. Ofran Y, Tallman M S, Rowe J M. How I treat acute myeloid leukemia presenting with preexisting comorbidities. Blood. 2016; 128(4):488-496.
5. Rowe J M, Tallman M S. How I treat acute myeloid leukemia. Blood. 2010; 116(17):3147-3156.
6. Dombret H, Seymour J F, Butrym A, et al. International phase 3 study of azacitidine vs conventional care regimens in older patients with newly diagnosed AML with >30% blasts. Blood. 2015; 126(3):291-299.
7. Fenaux P, Mufti G J, Hellstrom-Lindberg E, et al. Azacitidine prolongs overall survival compared with conventional care regimens in elderly patients with low bone marrow blast count acute myeloid leukemia. J Clin Oncol. 2010; 28(4):562-569.
8. Guillermo Garcia-Manero, Ehab Atallah, Samer K Khaled, et al. Final results from a phase 2 study of pracinostat in combination with azacitidine in elderly patients with acute myeloid leukemia (AML). Blood. 2015; 126:453.
9. Swords R T, Erba H P, DeAngelo D J, et al. Pevonedistat (MLN4924), a First-in-Class NEDD8-activating enzyme inhibitor, in patients with acute myeloid leukaemia and myelodysplastic syndromes: a phase 1 study. Br J Haematol. 2015; 169(4):534-543.
10. Brownell J E, Sintchak M D, Gavin J M, et al. Substrate-assisted inhibition of ubiquitin-like protein-activating enzymes: the NEDD8 E1 inhibitor MLN4924 forms a NEDD8-AMP mimetic in situ. Mol Cell. 2010; 37(1):102-111.
11. Milhollen M A, Traore T, Adams-Duffy J, et al. MLN4924, a NEDD8-activating enzyme inhibitor, is active in diffuse large B-cell lymphoma models: rationale for treatment of NF-κB-dependent lymphoma. Blood. 2010; 116(9):1515-1523.
12. Soucy T A, Smith P G, Milhollen M A, et al. An inhibitor of NEDD8-activating enzyme as a new approach to treat cancer. Nature. 2009; 458(7239):732-736.
13. Chen G, Li G. Increased Cull expression promotes melanoma cell proliferation through regulating p27 expression. Int J Oncol. 2010; 37(5):1339-1344.
14. Chiba T, Tanaka K. Cullin-based ubiquitin ligase and its control by NEDD8-conjugating system. Curr Protein Pept Sci. 2004; 5(3):177-184.
15. Crusio K M, King B, Reavie L B, Aifantis I. The ubiquitous nature of cancer: the role of the SCF(Fbw7) complex in development and transformation. Oncogene. 2010; 29(35):4865-4873.
16. Gstaiger M, Jordan R, Lim M, et al. Skp2 is oncogenic and overexpressed in human cancers. Proc Natl Acad Sci USA. 2001; 98(9):5043-5048.
17. Herrmann J, Lerman L O, Lerman A. Ubiquitin and ubiquitin-like proteins in protein regulation. Circ Res. 2007; 100(9):1276-1291.
18. Swords R T, Kelly K R, Smith P G, et al. Inhibition of NEDD8-activating enzyme: a novel approach for the treatment of acute myeloid leukemia. Blood. 2010; 115(18):3796-3800.
19. Smith P G, Traore T, Grossman S, et al. Azacitidine/Decitabine Synergism with the NEDD8-Activating Enzyme Inhibitor MLN4924 in Pre-Clinical AML Models. Blood. 2011; 118(21):267-267. Abstract 615.
20. National Cancer Institute Cancer Therapy Evaluation Program (NCI CTEP). National Cancer Institute Common Terminology Criteria for Adverse Events (NCI-CTCAE). Version 3, 9 Aug. 2006, 2011.
21. Cheson B D, Bennett J M, Kopecky K J, et al. Revised recommendations of the international working group for diagnosis, standardization of response criteria, treatment outcomes, and reporting standards for therapeutic trials in acute myeloid leukemia. J Clin Oncol. 2003; 21(24):4642-4649.
22. Swords R T, Watts J, Erba H P, et al. Expanded safety analysis of pevonedistat, a first-in-class NEDD8-activating enzyme inhibitor, in patients with acute myeloid leukemia and myelodysplastic syndromes. Blood Cancer J 2017; 7(2):e520.
23. Papaemmanuil E, Gerstung M, Bullinger L, et al. Genomic classification and prognosis in acute myeloid leukemia. New England Journal of Medicine. 2016; 374(23):2209-2221.
24. O'Donnell M R, Abboud C N, Altman J, et al. NCCN Clinical Practice Guidelines Acute myeloid leukemia. J Natl Compr Canc Netw. 2012; 10(8):984-1021.
25. Pleyer L, Burgstaller S, Girschikofsky M, et al. Azacitidine in 302 patients with WHO-defined acute myeloid leukemia: results from the Austrian Azacitidine Registry of the AGMT-Study Group. Ann Hematol. 2014; 93(11):1825-1838.
26. Cooper B W, Kindwall-Keller T L, Craig M D, et al. A phase I study of midostaurin and azacitidine in relapsed and elderly AML patients. Clin Lymphoma Myeloma Leuk. 2015; 15(7):428-432.
27. Kenealy M, Patton N, Filshie R, et al. Results of a phase II study of thalidomide and azacitidine in patients with clinically advanced myelodysplastic syndromes (MDS), chronic myelomonocytic leukemia (CMML) and low blast count acute myeloid leukemia (AML). Leuk Lymphoma. 2017; 58(2):298-307.
28. Ramsingh G, Westervelt P, Cashen A F, et al. A phase 1 study of concomitant high-dose lenalidomide and 5-azacitidine induction in the treatment of AML. Leukemia. 2013; 27(3):725-728.
29. S. Bhatia, O. Hamid, A. C. Pavlick, G. Mulligan, P. G. Smith, M. D. Pickard. MLN4924, an investigational NEDD8-activating enzyme (NAE) inhibitor, in patients (pts) with metastatic melanoma: Results of a phase I study. J Clin Oncol. 2011; 29:Abstract 8529.
30. Czuczman N M, Barth M J, Gu J, et al. Pevonedistat, a NEDD8-activating enzyme inhibitor, is active in mantle cell lymphoma and enhances rituximab activity in vivo. Blood. 2016; 127(9):1128-1137.
31. Harvey R. MLN4924, an investigational NEDD8-activating (NAE) inhibitor, inpatients with relapsed and/or refractory lymphoma or multiple myeloma (MM): Phase I dose escalation study of twice-weekly dosing. European Hematology Association. 2012:Abstract 1060.
32. Kauh Jea. Pharmacodynamic effects of MLN4924, a novel NAE inhibitor, on blood and skin. J Clin Oncol 27:15s (suppl: abstr 2562). 2009.
33. Kauh J. MLN4924, an investigational NEDD8-activating enzyme (NAE) inhibitor, in patients (pts) with advanced solid tumors: Phase I study of multiple treatment schedules. 2011.
34. Lin J J, Milhollen M A, Smith P G, Narayanan U, Dutta A. NEDD8-targeting drug MLN4924 elicits DNA rereplication by stabilizing Cdtl in S phase, triggering checkpoint activation, apoptosis, and senescence in cancer cells. Cancer Res. 2010; 70(24):10310-10320.
35. Milhollen M A, Narayanan U, Soucy T A, Veiby P O, Smith P G, Amidon B. Inhibition of NEDD8-activating enzyme induces rereplication and apoptosis in human tumor cells consistent with deregulating CDT1 turnover. Cancer Res. 2011; 71(8):3042-3051.
36. Sarantopoulos J, Shapiro G I, Cohen R B, et al. Phase I Study of the Investigational NEDD8-Activating Enzyme Inhibitor Pevonedistat (TAK-924/MLN4924) in Patients with Advanced Solid Tumors. Clin Cancer Res. 2016; 22(4):847-857.
37. Shah J. Phase 1 Dose Escalation Study of MLN4924, a Novel NAE Inhibitor, in Patients with Multiple Myeloma and Non-Hodgkin Lymphoma. American Society of Hematology. 2009:Abstract 1854.
38. Shah J J, Jakubowiak A J, O'Connor O A, et al. Phase I Study of the Novel Investigational NEDD8-Activating Enzyme Inhibitor Pevonedistat (MLN4924) in Patients with Relapsed/Refractory Multiple Myeloma or Lymphoma. Clin Cancer Res. 2016; 22(1):34-43.
39. Soucy T A, Dick L R, Smith P G, Milhollen M A, Brownell J E. The NEDD8 Conjugation Pathway and Its Relevance in Cancer Biology and Therapy. Genes Cancer. 2010; 1(7):708-716.
40. Soucy T A, Smith P G, Rolfe M. Targeting NEDD8-activated cullin-RING ligases for the treatment of cancer. Clin Cancer Res. 2009; 15(12):3912-3916.
41. Traore Tea. Antitumor activity of MLN4924, an investigational inhibitor of NEDD8-activating enzyme (NAE), in preclinical models of melanoma. J Clin Oncol 29: (suppl; abstr 8594). 2010.
42. Wang M, Medeiros B C, Erba H P, DeAngelo D J, Giles F J, Swords R T. Targeting protein neddylation: a novel therapeutic strategy for the treatment of cancer. Expert Opin Ther Targets. 2011; 15(3):253-264.
43. El-Mesery M, Seher A, Stuhmer T, Siegmund D, Wajant H. MLN4924 sensitizes monocytes and maturing dendritic cells for TNF-dependent and -independent necroptosis. Br J Pharmacol. 2015; 172(5):1222-1236.
44. Garcia K, Blank J L, Bouck D C, et al. Nedd8-activating enzyme inhibitor MLN4924 provides synergy with mitomycin C through interactions with ATR, BRCA1/BRCA2, and chromatin dynamics pathways. Mol Cancer Ther. 2014; 13(6):1625-1635.
45. Ho I L, Kuo K L, Liu S H, et al. MLN4924 synergistically enhances cisplatin-induced cytotoxicity via INK and Bcl-xL pathways in human urothelial carcinoma. Sci Rep. 2015; 5:16948.
46. Jazaeri A A, Shibata E, Park J, et al. Overcoming platinum resistance in preclinical models of ovarian cancer using the neddylation inhibitor MLN4924. Mol Cancer Ther. 2013; 12(10):1958-1967.
47. Khalife J, Radomska H S, Santhanam R, et al. Pharmacological targeting of miR-155 via the NEDD8-activating enzyme inhibitor MLN4924 (Pevonedistat) in FLT3-ITD acute myeloid leukemia. Leukemia. 2015; 29(10):1981-1992.
48. Knorr K L, Schneider P A, Meng X W, et al. MLN4924 induces Noxa upregulation in acute myelogenous leukemia and synergizes with Bcl-2 inhibitors. Cell Death Differ. 2015; 22(12):2133-2142.
49. Lin W C, Kuo K L, Shi C S, et al. MLN4924, a Novel NEDD8-activating enzyme inhibitor, exhibits antitumor activity and enhances cisplatin-induced cytotoxicity in human cervical carcinoma: in vitro and in vivo study. Am J Cancer Res. 2015; 5(11):3350-3362.
50. Nawrocki S T. Disrupting NEDD8-mediated protein turnover with MLN4924 significantly augments the efficacy of cytarabine. Blood. 2010; 116:1335a, (suppl; abstr 3255).
51. Nawrocki S T, Griffin P, Kelly K R, Carew J S. MLN4924: a novel first-in-class inhibitor of NEDD8-activating enzyme for cancer therapy. Expert Opin Investig Drugs. 2012; 21(10):1563-1573.
52. Nawrocki S T, Kelly K R, Smith P G, et al. Disrupting protein NEDDylation with MLN4924 is a novel strategy to target cisplatin resistance in ovarian cancer. Clin Cancer Res. 2013; 19(13):3577-3590.
53. Oladghaffari M, Islamian J P, Baradaran B, et al. High Efficiency Apoptosis Induction in Breast Cancer Cell Lines by MLN4924/2D G Co-Treatment. Asian Pac J Cancer Prev. 2015; 16(13):5471-5476.
54. Sumi H, Inazuka M, Morimoto M, et al. An inhibitor of apoptosis protein antagonist T-3256336 potentiates the antitumor efficacy of the Nedd8-activating enzyme inhibitor pevonedistat (TAK-924/MLN4924). Biochem Biophys Res Commun. 2016; 480(3):380-386.
55. Visconte V, Nawrocki S T, Espitia C M, et al. Comprehensive quantitative proteomic profiling of the pharmacodynamic changes induced by MLN4924 in acute myeloid leukemia cells establishes rationale for its combination with azacitidine. Leukemia. 2016; 30(5):1190-1194.
56. Wan J, Zhu J, Li G, Zhang Z. Radiosensitization of Human Colorectal Cancer Cells by MLN4924: An Inhibitor of NEDD8-Activating Enzyme. Technol Cancer Res Treat. 2016; 15(4):527-534.
57. Wei D, Li H, Yu J, et al. Radiosensitization of human pancreatic cancer cells by MLN4924, an investigational NEDD8-activating enzyme inhibitor. Cancer Res. 2012; 72(1):282-293.
58. Zhao L, Yue P, Lonial S, Khuri F R, Sun S Y. The NEDD8-activating enzyme inhibitor, MLN4924, cooperates with TRAIL to augment apoptosis through facilitating c-FLIP degradation in head and neck cancer cells. Mol Cancer Ther. 2011; 10(12):2415-2425.
59. Zhou L, Chen S, Zhang Y, et al. The NAE inhibitor pevonedistat interacts with the HDAC inhibitor belinostat to target AML cells by disrupting the DDR. Blood. 2016.
60. Wolenski Fea. The NAE Inhibitor MLN4924 Synergizes with TNF-α to Induce Death Through Apoptosis and Necroptosis in a Rat Hepatoma Cell Line. August 11-16, Andover, N.H., poster presentation). 2013.
61. Celgene Corporation. VIDAZA Prescribing Information, updated 2016. Available from: the media portion of the celgene website.
62. Mikkael S, Megan O, Alan F, et al. A Randomized Phase II Study of Azacitidine Combined with Lenalidomide or with Vorinostat Vs. Azacitidine Monotherapy in Higher-Risk Myelodysplastic Syndromes (MDS) and Chronic Myelomonocytic Leukemia (CMML): North American Intergroup Study SWOG S1117. Blood. 2014; 124:LBA-5.
63. Hou H A, Chou W C, Kuo Y Y, et al. TP53 mutations in de novo acute myeloid leukemia patients: longitudinal follow-ups show the mutation is stable during disease evolution. Blood Cancer Journal. 2015; 5.
64. Welch J S, Petti A A, Miller C A, et al. TP53 and Decitabine in Acute Myeloid Leukemia and Myelodysplastic Syndromes. N Engl J Med. 2016; 375(21):2023-2036.

### Example 2. Pevonedistat-Azacitidine Mutation-Based Predictive Model

A decision tree model was developed to classify patients who respond to pevonedistat plus azacitidine treatment in myelodysplastic disorders based on their mutation profiles, and to use this model to predict treatment-responsive patients a priori. The decision tree model was developed with patient response data and short-range mutation data from a 116 gene custom panel (HemeOnc V1.1) in the clinical study described in Example 1. The decision tree model was approved for testing in the phase 2 clinical study (NCT02610777, An Efficacy and Safety Study of Pevonedistat Plus Azacitidine Versus Single-Agent Azacitidine in Participants With Higher-Risk Myelodysplastic Syndromes (HR MDS), Chronic Myelomonocytic Leukemia (CMML) and Low-Blast Acute Myelogenous Leukemia (AML)).

The Varscan 2.0 algorithm called mutations in the 116 gene HemeOnc V1.1 panel from patient-derived DNA samples in the phase 1b study. Mutations were then annotated as cancer-driving. Because only a few genes are frequently mutated in myelodysplastic disorders, fourteen genes known to have high mutation rates in MDS highly were selected to develop a predictive model. The mutation matrix for these highly mutated genes is shown in Table 8. In Table 8, each row represents a patient in the phase 1b clinical study (described in Example 1), with the indicated response category (CR, PR, SD). Boxes populated with a “1” indicate that a mutation was detected in a biological sample, boxes populated with a “0” indicate that no mutation was detected. A gene was called ‘mutated’ if a known cancer-driving mutation was detected.

TABLE 8
Highly Mutated Gene Matrix.

| Gene | PHF6 | TET2 | EZH2 | NRAS | KRAS | MPL | SF3B1 | TP53 | DNMT3 | RUNX1 | IDH2 | ASXL1 | FLT3 | IDH1 |
|---|---|---|---|---|---|---|---|---|---|---|---|---|---|---|
| CR1 | 0 | 1 | 0 | 0 | 0 | 1 | 0 | 0 | 0 | 0 | 0 | 0 | 0 | 0 |
| CR2 | 0 | 0 | 0 | 0 | 0 | 0 | 0 | 0 | 0 | 0 | 0 | 0 | 0 | 0 |
| CR3 | 0 | 1 | 0 | 0 | 0 | 0 | 0 | 0 | 0 | 0 | 0 | 0 | 0 | 0 |
| CR4 | 0 | 0 | 0 | 1 | 0 | 0 | 0 | 0 | 0 | 0 | 0 | 0 | 0 | 0 |
| CR5 | 0 | 0 | 0 | 0 | 0 | 0 | 0 | 0 | 0 | 1 | 0 | 0 | 0 | 0 |
| CR6 | 0 | 0 | 0 | 0 | 1 | 0 | 0 | 0 | 1 | 0 | 0 | 0 | 0 | 0 |
| CR7 | 0 | 1 | 0 | 0 | 0 | 0 | 0 | 0 | 0 | 0 | 0 | 0 | 0 | 0 |
| CR8 | 0 | 0 | 0 | 1 | 1 | 0 | 0 | 0 | 1 | 0 | 0 | 0 | 0 | 0 |
| CR9 | 0 | 0 | 0 | 1 | 0 | 0 | 1 | 0 | 0 | 0 | 0 | 0 | 0 | 0 |
| CR10 | 0 | 0 | 0 | 0 | 0 | 0 | 0 | 0 | 0 | 0 | 0 | 0 | 0 | 0 |
| CRi1 | 0 | 0 | 0 | 0 | 0 | 0 | 0 | 0 | 0 | 1 | 1 | 0 | 0 | 0 |
| CRi2 | 0 | 0 | 1 | 1 | 1 | 0 | 0 | 0 | 1 | 0 | 0 | 0 | 0 | 0 |
| CRi3 | 0 | 0 | 0 | 0 | 0 | 0 | 0 | 1 | 0 | 0 | 0 | 0 | 0 | 0 |
| PR1 | 0 | 0 | 0 | 0 | 0 | 0 | 0 | 0 | 0 | 1 | 1 | 0 | 0 | 0 |
| PR2 | 0 | 1 | 0 | 0 | 0 | 0 | 0 | 0 | 0 | 0 | 0 | 0 | 0 | 0 |
| PR3 | 0 | 0 | 0 | 0 | 0 | 0 | 0 | 1 | 0 | 0 | 0 | 0 | 0 | 0 |
| PR4 | 0 | 0 | 0 | 0 | 0 | 0 | 0 | 1 | 0 | 0 | 0 | 0 | 0 | 0 |
| PR5 | 0 | 0 | 0 | 0 | 0 | 0 | 0 | 0 | 1 | 0 | 0 | 0 | 0 | 0 |
| PR6 | 1 | 0 | 0 | 0 | 0 | 0 | 0 | 1 | 0 | 0 | 0 | 0 | 0 | 0 |
| SD1 | 0 | 0 | 0 | 0 | 0 | 0 | 0 | 0 | 0 | 0 | 0 | 1 | 0 | 0 |
| SD2 | 0 | 0 | 0 | 0 | 0 | 0 | 0 | 0 | 0 | 1 | 0 | 0 | 1 | 0 |
| SD3 | 0 | 0 | 0 | 0 | 0 | 0 | 0 | 1 | 0 | 0 | 0 | 0 | 0 | 0 |
| SD4 | 0 | 0 | 0 | 0 | 0 | 0 | 0 | 0 | 0 | 0 | 0 | 0 | 0 | 0 |
| SD5 | 0 | 0 | 0 | 0 | 0 | 0 | 0 | 0 | 0 | 0 | 0 | 0 | 0 | 0 |
| SD6 | 0 | 0 | 0 | 0 | 0 | 0 | 0 | 0 | 0 | 0 | 1 | 0 | 0 | 0 |
| SD7 | 0 | 0 | 0 | 0 | 0 | 0 | 0 | 0 | 0 | 0 | 0 | 0 | 0 | 0 |
| SD8 | 0 | 0 | 0 | 0 | 0 | 0 | 0 | 0 | 0 | 0 | 0 | 0 | 0 | 0 |
| SD9 | 0 | 0 | 0 | 0 | 0 | 0 | 0 | 0 | 1 | 0 | 0 | 1 | 0 | 1 |
| PD1 | 0 | 0 | 0 | 0 | 0 | 0 | 0 | 0 | 0 | 0 | 1 | 0 | 0 | 0 |

Starting with 14 highly mutated genes, a regression tree model was developed with a recursive partitioning algorithm from the R programming language package ‘rpart’. The 14 genes were first ordered by a Fisher's exact test p-value measuring association with trial categorical outcome. Genes were added 1 at a time until the performance of the decision tree reached a plateau (9 genes). Then the final decision tree was created with 9 genes as input. One gene was pruned to create the final model. Because of the mutually exclusive nature of some mutations, the decision tree can also be expressed as a rule: [ASXL1wt And IDH1wt] And [TET2mut Or NRASmut Or KRASmut Or DNMT3Amut Or TP53mut Or RunXlmut]. This rule can be interpreted as: A patient with a mutation in TET2, NRAS, KRAS, DNMT3A, TP53 or RUNX1 and no mutations in ASXL1 or IDH1 will respond to pevonedistat plus azacitidine. The model was validated on a phase 2 trial independent testing dataset.

### Example 3. Isolation of Nucleic Acid and Nucleic Acid Sequencing Methods

Genomic Isolations and DNA Sequencing

DNA isolation from cells and tumors is conducted using DNAEASY® isolation kit (Qiagen, Valencia, Calif.). RNA isolation is conducted using MegaMax (Ambion division of Applied Biosystems, Austin, Tex.). Genomic isolations are conducted following manufacturer recommend protocols.

Sanger Sequencing Methodology.

PCR amplifications are conducted using optimized cycling conditions per gene-exon. Primer extension sequencing is performed using Applied Biosystems BigDye version 3.1. The reactions are then run on Applied Biosystem's 3730xl DNA Analyzer. Sequencing base calls are done using KB™ Basecaller (Applied Biosystems). Somatic Mutation calls are determined by Mutation Surveyor (SoftGenetics) and confirmed manually by aligning sequencing data with the corresponding reference sequence using Seqman (DNASTAR).

Sequenom Sequencing Methodology.

Sequenom (San Diego, Calif.) assays are designed using TypePLEX® chemistry with single-base extension. This process consists of three steps: 1) A text file containing the SNPs or mutations of interest and flanking sequence is uploaded at mysequenom.com where it is run through a web based program ProxSNP, 2) The output of ProxSNP is run through PreXTEND and 3) the output of PreXTEND is run through Assay Design which determines the expected mass weight of the extend products to ensure separation between all potential peaks found within a multiplexed reaction.

PCR primers are then designed to bracket the region identified in the assay design steps. The region of interest is amplied in PCR reactions using the primers. 15 nl of amplified and extended product is spotted on a 384 SpectroCHIP II using a Nanodispenser RS1000. A 3-point calibrant is added to every chip to ensure proper performance of the Sequenom Maldi-tof compact mass spectrometer.

The SpectroCHIP II is placed in the Sequenom MALDI-TOF compact mass spectrometer. The mass spectrometer is set to fire a maximum of 9 acquisitions for each spot on the 384 well spectroCHIP. TypePLEX Gold kit SpectroCHIP II from Sequenom (10142-2) is used following manufacturers recommended protocols. Analysis is performed using Sequenom analysis software, MassARRAY® Typer Analyzer v4.

Next Generation Sequencing (NGS) Methodology.

Targeted NGS using the Illumina platform (Illumina, Inc. San Diego, Calif.) is used to confirm and identify low frequency mutations in a marker. Primer pairs are designed to amplify coding exons. PCR products are quantified using a PicoGreen assay and combined in equal molar ratios for each sample. The purified products are end-repaired and concatenated by ligation. The concatenated products are used for Hi-Seq 2000 library preparation. The concatenated PCR products are sheared and used to make barcoded Hi-Seq 2000 libraries consisting of 12 barcoded samples per multiplexed pool. The pooled Hi-Seq 2000 libraries undergo clonal amplification by cluster generation on eight lanes of a Hi-Seq 2000 flow cell and are sequenced using 1×100 single-end sequencing on a Hi-Seq 2000. Matching of primary sequencing reads to the human genome build Hg18, as well as SNP analysis are performed using Illumina's CASAVA software version 1.7.1.

### General Procedures

### Quantitative RT-PCR

cDNA synthesis and quantitative RT-PCR is performed using ABI Gene Expression Assays, reagents, and ABI PRISM® 7900HT Sequence Detection Systems (Applied Biosystems, Foster City, Calif.) using the following cycle conditions: hold at 50° C. for 2 minutes for AmpErase UNG activation, then 95.0° C. for 10 minutes to activate DNA polymerase then run 40 two-part cycles of 95.0° C. for 15 seconds and 60.0° C. for 1 minute. The dCt is calculated by using the average Ct of control genes B2M (Hs99999907_m1) and RPLPO (Hs99999902_m1). Relative mRNA expression quantification is derived using the Comparative Ct Method (Applied Biosystems). mRNA expression fold change values are generated from a normal sample and corresponding tumor sample.

### EQUIVALENTS

Although embodiments of the invention have been described using specific terms, such description are for illustrative purposes only, and it is to be understood that changes and variations may be made without departing from the spirit or scope of the invention. Those skilled in the art will recognize, or be able to ascertain using no more than routine experimentation, many equivalents of the specific embodiments of the invention described herein. Such equivalents are intended to be encompassed by the following claims.

What is claimed:

1. A method for treating a hematological cancer patient comprising a hematological tumor characterized by wild type additional sex combs-like 1 (ASXL1), wild-type isocitrate dehydrogenase (NADPH(+)) 1, cytosolic (IDH1), and at least one altered hematological cancer marker gene, comprising the step of administering to the patient a therapeutically effective amount of pevonedistat or a pharmaceutically acceptable salt thereof.

2. The method of claim 1, wherein the at least one altered hematological cancer marker gene is selected from the group consisting of ten-eleven translocation methylcytosine dioxygenase 2 (TET2), neuroblastoma RAS viral (v-ras) oncogene homolog (NRAS), v-Ki-ras2 Kirsten rat sarcoma viral oncogene homolog (KRAS), DNA methyltransferase 3 alpha (DNMT3A), tumor protein p53 (TP53) and runt-related transcription factor 1 (RUNX1).

3. The method of claim 1 or 2, further comprising administering a hypomethylating agent to the patient.

4. The method of claim 3, wherein the hypomethylating agent is azacitidine.

5. The method of any of claims 1 to 4, wherein the hematological cancer is selected from the group consisting of leukemia, lymphoma and myeloma.

6. The method of any of claims 1 to 5, wherein the hematological cancer is selected from the group consisting of acute myelogenous leukemia (AML), myelodysplastic syndrome (MDS) and chronic myelomonocytic leukemia (CMML).

7. The method of any of claims 1 to 6, further comprising the steps of:
a) measuring at least one characteristic of at least one marker corresponding to ASXL1 and IDH1 and a hematological cancer marker gene in a biological sample obtained from the patient;
b) identifying wild type ASXL1 and IDH1 and at least one altered hematological cancer marker gene in the hematological tumor from the at least one characteristic measured in step a); and
c) administering the pevonedistat or a pharmaceutically acceptable salt thereof to the patient.

8. The method of claim 7, wherein the at least one marker is selected from the group consisting of nucleic acid and protein corresponding to the at least one marker gene.

9. The method of claim 7 or 8, wherein the at least one characteristic is selected from the group consisting of size, sequence, composition, activity and amount.

10. The method of claim 9, wherein the at least one characteristic is sequence.

11. The method of any one of claims 8 to 10, wherein the at least one marker is nucleic acid.

12. The method of claim 11, wherein the nucleic acid is selected from the group consisting of DNA, mRNA and cDNA or a portion of any of the foregoing, wherein the portion comprises at least one mutation site of the at least one marker gene.

13. The method of any one of claims 1 to 12, wherein the pharmaceutically acceptable salt of pevonedistat is a hydrochloride salt.

14. An NAE inhibitor or a pharmaceutically acceptable salt thereof for use in a method of treating a hematological cancer in a patient whose hematological tumor is characterized by having wild type ASXL1 gene, wild type IDH1 gene, and at least one altered hematological cancer marker gene selected from the group consisting of TET2, NRAS, KRAS, DNMT3A, TP53 and RUNX1.

15. The NAE inhibitor or a pharmaceutically acceptable salt thereof for the use of claim 14, wherein the NAE inhibitor or pharmaceutically acceptable salt thereof is for use in combination with a hypomethylating agent.

16. The NAE inhibitor or pharmaceutically acceptable salt thereof for the use in the combination with a hypomethylating agent of claim 14 or 15, wherein the hypomethylating agent is azacitidine.

17. The NAE inhibitor or pharmaceutically acceptable salt thereof for the use in the combination with a hypomethylating agent of any one of claims 14 to 16, wherein the hematological cancer is selected from the group consisting of leukemia, lymphoma and myeloma.

18. The NAE inhibitor or pharmaceutically acceptable salt thereof for the use in the combination with a hypomethylating agent of any one of claims 14 to 17, wherein the hematological cancer is selected from the group consisting of acute myelogenous leukemia (AML), myelodysplastic syndrome (MDS), and chronic myelomonocytic leukemia (CMML).

19. The NAE inhibitor or pharmaceutically acceptable salt thereof for the use of any one of claims 14 to 18, wherein the NAE inhibitor is pevonedistat or a pharmaceutically acceptable salt thereof.

20. The NAE inhibitor or pharmaceutically acceptable salt thereof for the use of any one of claim 19, wherein the NAE inhibitor is pevonedistat hydrochloride.

21. A method for determining whether to treat a patient having a hematological cancer with a therapeutic regimen comprising an NEDD8-activating enzyme (NAE) inhibitor comprising:
a) measuring at least one characteristic of at least one marker corresponding with at least one marker gene in a biological sample obtained from the patient;
b) identifying mutational status of the marker gene from the measurement in step a); and
c) determining to treat the patient with the therapeutic regimen if the mutational status indicates a favorable outcome,
wherein the at least one marker gene is selected from the group consisting of TET2, RUNX1, NRAS, KRAS, and DNMT3A.

22. The method of claim 21, wherein one or more marker gene selected from the group consisting of TET2, RUNX1, NRAS, KRAS, and DNMT3A is an altered hematological cancer marker gene.

23. The method of claim 22, wherein the patient has a hematological cancer further characterized by wild type ASXL1 and wild type IDH1.

24. The method of any one of claims 21 to 23, wherein the at least one characteristic is selected from the group consisting of size, sequence, composition and amount.

25. The method of any one of claims 22 to 24, wherein the alteration in the marker gene is an inactivating mutation.

26. The method of any one of claims 21 to 25, wherein the at least one marker is selected from the group consisting of nucleic acid and protein corresponding to the marker gene.

27. The method of any one of claims 21 to 26, wherein the hematological cancer is selected from the group consisting of myeloma, leukemia, lymphoma, and myelodysplastic syndrome.

28. The method of any one of claims 21 to 27, wherein the hematological cancer is selected from the group consisting of acute myelogenous leukemia, chronic myelomonocytic leukemia, and myelodysplastic syndrome.

29. The method of claim 28, wherein the hematological cancer is selected from the group consisting of low blast acute myelogenous leukemia, high risk myelodysplastic syndrome, and chronic myelomonocytic leukemia.

30. The method of any one of claims 21 to 29, wherein the biological sample comprises tumor cells, contents from the tumor cells or products from the tumor cells.

31. The method claim 29 or 30 wherein the at least one marker gene is at least two marker genes.

32. The method of any one of claims 21 to 30, wherein the at least one marker gene is TET2.

33. The method of any one of claims 21 to 30, wherein the at least one marker gene is RUNX1.

34. The method of any one of claims 21 to 30, wherein the at least one marker gene is NRAS.

35. The method of any one of claims 21 to 30, wherein the at least one marker gene is KRAS.

36. The method of any one of claims 21 to 30, wherein the at least one marker gene is DNMT3A.

37. The method of any of claims 31 to 36, wherein at least one second marker gene is selected from the group consisting of TP53, IDH2, EZH2, IDH1, NPM1, PHF6, and ASXL1 is measured.

38. The method of claim 37, wherein at least one second marker gene is ASXL1.

39. The method of claim 37, wherein at least one second marker gene is ASXL1 and IDH1.

40. The method of any one of claims 21 to 39, wherein the at least one characteristic is sequence of at least one marker.

41. The method of claim 40, wherein the at least one marker is a nucleic acid.

42. The method of claim 41, wherein the nucleic acid is selected from the group consisting of DNA, mRNA and cDNA or any portion of any of the foregoing, wherein the portion corresponds to at least one mutation site of the at least one marker gene.

43. The method of any one of claims 21 to 42, wherein the NAE inhibitor is pevonedistat or a pharmaceutically acceptable salt thereof.

44. The method of claim 43, wherein the pharmaceutically acceptable salt is a hydrochloride salt.

45. The method of any one of claims 21 to 44, wherein the therapeutic regimen further comprises administering a hypomethylating agent.

46. The method of claim 45, wherein the hypomethylating agent is azacitidine.

47. A method for determining whether to continue treatment of hematological cancer in a patient with a therapeutic regimen comprising an NAE inhibitor comprising:
a) obtaining a first biological sample from the patient and a second biological sample from the patient, wherein the first sample is obtained prior to the second sample and the patient is treated with the therapeutic regimen prior to the second sample;
b) measuring at least one characteristic of at least one marker in the two samples;
c) comparing the results of the measurements in b); and
d) determining to continue treatment with the therapeutic regimen if the comparison indicates that the tumor cells in the second sample comprise at least one marker gene whose mutational status indicates a favorable outcome,
wherein the at least one marker gene is selected from the group consisting of TET2, RUNX1, NRAS, KRAS, and DNMT3A.

48. The method of claim 47, wherein the at least one characteristic is selected from the group consisting of size, sequence, composition and amount.

49. The method of claim 47 or 48, wherein the mutational status of the at least one marker gene is mutant.

50. The method of claim 47 or 48, wherein the mutational status of the at least one marker gene is wild type.

51. The method of any one of claims 47 to 50, wherein the at least one marker is selected from the group consisting of nucleic acid and protein corresponding to the at least one marker gene.

52. The method of any one of claims 47 to 51, wherein the hematological cancer is selected from the group consisting of myeloma, leukemia, lymphoma, and myelodysplastic syndrome.

53. The method of any one of claims 47 to 51, wherein the hematological cancer is selected from the group consisting of acute myelogenous leukemia, chronic myelomonocytic leukemia, and myelodysplastic syndrome.

54. The method of any one of claims 47 to 51, wherein the hematological cancer is selected from the group consisting of low blast acute myelogenous leukemia, high risk myelodysplastic syndrome, and chronic myelomonocytic leukemia.

55. The method of any one of claims 47 to 54, wherein the biological sample comprises hematological tumor cells.

56. The method of any one of claims 47 to 55, wherein the at least one marker gene is at least two marker genes.

57. The method of claim 56, wherein at least two marker genes are ASXL1 and IDH1.

58. The method of any one of claims 47 to 57, wherein the at least one characteristic is sequence of at least one marker.

59. The method of any one of claims 47 to 57, wherein the at least one marker is a nucleic acid.

60. The method of claim 59, wherein the nucleic acid is selected from the group consisting of DNA, mRNA and cDNA or any portion of any of the foregoing, wherein the portion corresponds to at least one mutation site of the at least one marker gene.

61. The method of any one of claims 47-60, wherein the NAE inhibitor is pevonedistat or a pharmaceutically acceptable salt thereof.

62. The method of claim 61, wherein the pharmaceutically acceptable salt is a hydrochloride salt.

63. The method of any one of claims 47 to 62, wherein the therapeutic regimen further comprises administering a hypomethylating agent.

64. The method of claim 63, wherein the hypomethylating agent is azacitidine.

65. A kit comprising a reagent to measure at least one characteristic of at least one marker in a biological sample, wherein the at least one marker corresponds to at least one marker gene is selected from the group consisting of TET2, RUNX1, NRAS, KRAS, and DNMT3A.

66. A kit comprising a reagent to measure at least one characteristic of the markers in a biological sample in the method of any of claims 1-13.

67. The kit of claim 65 or 66, wherein the at least one characteristic is selected from the group consisting of size, sequence, composition and amount.

68. The kit of any one of claims 65 to 67, wherein the at least one marker is selected from the group consisting of nucleic acid and protein corresponding to the at least one marker gene.

69. The kit of any one of claims 65 to 68, further comprising a stabilizer to add to the sample.

70. The kit of any one of claims 65 to 69, wherein the at least one marker is nucleic acid and the reagent is at least one primer.

71. The kit of any one of claims 65 to 70, further comprising a probe.

72. The kit of any one of claims 65 to 71, wherein the biological sample comprises hematological tumor cells.

73. The kit of claim 72, wherein the biological sample is blood.

74. The kit of claim 72 or 73, further comprising enriching the biological sample for tumor cells.

75. A method for paying for the treatment of a patient with hematological cancer with an NAE inhibitor comprising:
a) recording the mutational status of at least one marker gene in a biological sample comprising tumor cells, contents from the tumor cells or products from the tumor cells from the patient, and
b) authorizing payment of the NAE inhibitor treatment if the mutational status indicates a favorable outcome,
wherein the at least one marker gene is selected from the group consisting of TET2, RUNX1, NRAS, KRAS, and DNMT3A.

76. The method of claim 75, wherein the mutational status of the marker gene is mutant.

77. The method of claim 75, wherein the mutational status of the marker gene is wild type.

78. A method for treating a patient having a hematological cancer comprising administering a therapeutically effective amount of an NAE inhibitor to a patient having at least one marker gene whose mutational status indicates a favorable outcome to NAE inhibition therapy, wherein the at least one marker gene is selected from the group consisting of TET2, RUNX1, NRAS, KRAS, and DNMT3A.

79. The method of claim 78, wherein the mutational status of the at least one marker gene is mutant.

80. The method of claim 78, wherein the mutational status of the at least one marker gene is wild type.

81. The method of claim 79, wherein the mutation in the marker gene is an inactivating mutation.

82. The method of any one of claims 78 to 81, wherein the hematological cancer is myeloma, leukemia, lymphoma, or myelodysplastic syndrome.

83. The method of any one of claims 78 to 81, wherein the hematological cancer is acute myelogenous leukemia, chronic myelomonocytic leukemia, or myelodysplastic syndrome.

84. The method of any one of claims 78 to 81, wherein the hematological cancer is low blast acute myelogenous leukemia, high risk myelodysplastic syndrome, or chronic myelomonocytic leukemia.

85. The method of any one of claims 78 to 84, wherein the at least one marker gene is at least two marker genes.

86. The method of any one of claims 78 to 84, wherein the at least one marker gene is TET2.

87. The method of any one of claims 78 to 84, wherein the at least one marker gene is RUNX1.

88. The method of any one of claims 78 to 84, wherein the at least one marker gene is NRAS.

89. The method of any one of claims 78 to 84, wherein the at least one marker gene is KRAS.

90. The method of any one of claims 78 to 84, wherein the at least one marker gene is DNMT3A.

91. The method of any one of claims 78 to 90, wherein at least one second marker gene is selected from the group consisting of TP53, IDH2, EZH2, IDH1, NPM1, PHF6, and ASXL1 is measured.

92. The method of claim 91, wherein at least one second marker gene is ASXL1.

93. The method of claim 92, wherein at least one second marker gene is ASXL1 and IDH1.

94. The method of any one of claims 75 to 93, wherein the hematological cancer is characterized by wild type ASXL1 and wild type IDH1.

95. The method of claim 94, wherein the hematological cancer is characterized by at least one altered hematological cancer marker gene selected from the group consisting of TET2, NRAS, KRAS, DNMT3A, TP53 or RUNX1.

96. The method of any of claims 78 to 95, wherein the NAE inhibitor is pevonedistat or a pharmaceutically acceptable salt thereof.

97. The method of claim 96, wherein the pharmaceutically acceptable salt is a hydrochloride salt.

98. The method of any one of claims 78 to 97, wherein the therapeutically effective amount of the NAE inhibitor is about 15 mg/m² to about 40 mg/m².

99. The method of any one of claims 78 to 98, wherein the method further comprises administering a therapeutically effective amount of a hypomethylating agent.

100. The method of claim 99, wherein the hypomethylating agent is azacitidine.

101. The method of claim 99 or 100, wherein the therapeutically effective amount of the hypomethylating agent is about 75 mg/m².


SEQUENCE LISTING (24 sequences)

SEQ ID NO: 1 - DNA, 9796 bp, Homo sapiens
ggcagtggcagcggcgagagcttgggcggccgccgccgcctcctcgcgagcgccgcgcgcccgggtcccgctcgcatgcaagtcacgtccgccccctcggcgcggccgccccgagacgccggccccgctgagtgatgagaacagacgtcaaactgccttatgaatattgatgcggaggctaggctgctttcgtagagaagcagaaggaagcaagatggctgccctttaggatttgttagaaaggagacccgactgcaactgctggattgctgcaaggctgagggacgagaacgaggctggcaaacattcagcagcacaccctctcaagattgtttacttgcctttgctcctgttgagttacaacgcttggaagcaggagatgggctcagcagcagccaataggacatgatccaggaagagcagtaagggactgagctgctgaattcaactagagggcagccttgtggatggccccgaagcaagcctgatggaacaggatagaaccaaccatgttgagggcaacagactaagtccattcctgataccatcacctcccatttgccagacagaacctctggctacaaagctccagaatggaagcccactgcctgagagagctcatccagaagtaaatggagacaccaagtggcactctttcaaaagttattatggaataccctgtatgaagggaagccagaatagtcgtgtgagtcctgactttacacaagaaagtagagggtattccaagtgtttgcaaaatggaggaataaaacgcacagttagtgaaccttctctctctgggctccttcagatcaagaaattgaaacaagaccaaaaggctaatggagaaagacgtaacttcggggtaagccaagaaagaaatccaggtgaaagcagtcaaccaaatgtctccgatttgagtgataagaaagaatctgtgagttctgtagcccaagaaaatgcagttaaagatttcaccagtttttcaacacataactgcagtgggcctgaaaatccagagcttcagattctgaatgagcaggaggggaaaagtgctaattaccatgacaagaacattgtattacttaaaaacaaggcagtgctaatgcctaatggtgctacagtttctgcctcttccgtggaacacacacatggtgaactcctggaaaaaacactgtctcaatattatccagattgtgtttccattgcggtgcagaaaaccacatctcacataaatgccattaacagtcaggctactaatgagttgtcctgtgagatcactcacccatcgcatacctcagggcagatcaattccgcacagacctctaactctgagctgcctccaaagccagctgcagtggtgagtgaggcctgtgatgctgatgatgctgataatgccagtaaactagctgcaatgctaaatacctgttcctttcagaaaccagaacaactacaacaacaaaaatcagtttttgagatatgcccatctcctgcagaaaataacatccagggaaccacaaagctagcgtctggtgaagaattctgttcaggttccagcagcaatttgcaagctcctggtggcagctctgaacggtatttaaaacaaaatgaaatgaatggtgcttacttcaagcaaagctcagtgttcactaaggattccttttctgccactaccacaccaccaccaccatcacaattgcttctttctccccctcctcctcttccacaggttcctcagcttccttcagaaggaaaaagcactctgaatggtggagttttagaagaacaccaccactaccccaaccaaagtaacacaacacttttaagggaagtgaaaatagagggtaaacctgaggcaccaccttcccagagtcctaatccatctacacatgtatgcagcccttctccgatgctttctgaaaggcctcagaataattgtgtgaacaggaatgacatacagactgcagggacaatgactgttccattgtgttctgagaaaacaagaccaatgtcagaacacctcaagcataacccaccaatttttggtagcagtggagagctacaggacaactgccagcagttgatgagaaacaaagagcaagagattctgaagggtcgagacaaggagcaaacacgagatcttgtgcccccaacacagcactatctgaaaccaggatggattgaattgaaggcccctcgttttcaccaagcggaatcccatctaaaacgtaatgaggcatcactgccatcaattcttcagtatcaacccaatctctccaatcaaatgacctccaaacaatacactggaaattccaacatgcctggggggctcccaaggcaagcttacacccagaaaacaacacagctggagcacaagtcacaaatgtaccaagttgaaatgaatcaagggcagtcccaaggtacagtggaccaacatctccagttccaaaaaccctcacaccaggtgcacttctccaaaacagaccatttaccaaaagctcatgtgcagtcactgtgtggcactagatttcattttcaacaaagagcagattcccaaactgaaaaacttatgtccccagtgttgaaacagcacttgaatcaacaggcttcagagactgagccattttcaaactcacaccttttgcaacataagcctcataaacaggcagcacaaacacaaccatcccagagttcacatctccctcaaaaccagcaacagcagcaaaaattacaaataaagaataaagaggaaatactccagacttttcctcacccccaaagcaacaatgatcagcaaagagaaggatcattctttggccagactaaagtggaagaatgttttcatggtgaaaatcagtattcaaaatcaagcgagttcgagactcataatgtccaaatgggactggaggaagtacagaatataaatcgtagaaattccccttatagtcagaccatgaaatcaagtgcatgcaaaatacaggtttcttgttcaaacaatacacacctagtttcagagaataaagaacagactacacatcctgaactttttgcaggaaacaagacccaaaacttgcatcacatgcaatattttccaaataatgtgatcccaaagcaagatcttcttcacaggtgctttcaagaacaggagcagaagtcacaacaagcttcagttctacagggatataaaaatagaaaccaagatatgtctggtcaacaagctgcgcaacttgctcagcaaaggtacttgatacataaccatgcaaatgtttttcctgtgcctgaccagggaggaagtcacactcagacccctccccagaaggacactcaaaagcatgctgctctaaggtggcatctcttacagaagcaagaacagcagcaaacacagcaaccccaaactgagtcttgccatagtcagatgcacaggccaattaaggtggaacctggatgcaagccacatgcctgtatgcacacagcaccaccagaaaacaaaacatggaaaaaggtaactaagcaagagaatccacctgcaagctgtgataatgtgcagcaaaagagcatcattgagaccatggagcagcatctgaagcagtttcacgccaagtcgttatttgaccataaggctcttactctcaaatcacagaagcaagtaaaagttgaaatgtcagggccagtcacagttttgactagacaaaccactgctgcagaacttgatagccacaccccagctttagagcagcaaacaacttcttcagaaaagacaccaaccaaaagaacagctgcttctgttctcaataattttatagagtcaccttccaaattactagatactcctataaaaaatttattggatacacctgtcaagactcaatatgatttcccatcttgcagatgtgtagagcaaattattgaaaaagatgaaggtcctttttatacccatctaggagcaggtcctaatgtggcagctattagagaaatcatggaagaaaggtttggacagaagggtaaagctattaggattgaaagagtcatctatactggtaaagaaggcaaaagttctcagggatgtcctattgctaagtgggtggttcgcagaagcagcagtgaagagaagctactgtgtttggtgcgggagcgagctggccacacctgtgaggctgcagtgattgtgattctcatcctggtgtgggaaggaatcccgctgtctctggctgacaaactctactcggagcttaccgagacgctgaggaaatacggcacgctcaccaatcgccggtgtgccttgaatgaagagagaacttgcgcctgtcaggggctggatccagaaacctgtggtgcctccttctcttttggttgttcatggagcatgtactacaatggatgtaagtttgccagaagcaagatcccaaggaagtttaagctgcttggggatgacccaaaagaggaagagaaactggagtctcatttgcaaaacctgtccactcttatggcaccaacatataagaaacttgcacctgatgcatataataatcagattgaatatgaacacagagcaccagagtgccgtctgggtctgaaggaaggccgtccattctcaggggtcactgcatgtttggacttctgtgctcatgcccacagagacttgcacaacatgcagaatggcagcacattggtatgcactctcactagagaagacaatcgagaatttggaggaaaacctgaggatgagcagcttcacgttctgcctttatacaaagtctctgacgtggatgagtttgggagtgtggaagctcaggaggagaaaaaacggagtggtgccattcaggtactgagttcttttcggcgaaaagtcaggatgttagcagagccagtcaagacttgccgacaaaggaaactagaagccaagaaagctgcagctgaaaagctttcctccctggagaacagctcaaataaaaatgaaaaggaaaagtcagccccatcacgtacaaaacaaactgaaaacgcaagccaggctaaacagttggcagaacttttgcgactttcaggaccagtcatgcagcagtcccagcagccccagcctctacagaagcagccaccacagccccagcagcagcagagaccccagcagcagcagccacatcaccctcagacagagtctgtcaactcttattctgcttctggatccaccaatccatacatgagacggcccaatccagttagtccttatccaaactcttcacacacttcagatatctatggaagcaccagccctatgaacttctattccacctcatctcaagctgcaggttcatatttgaattcttctaatcccatgaacccttaccctgggcttttgaatcagaatacccaatatccatcatatcaatgcaatggaaacctatcagtggacaactgctccccatatctgggttcctattctccccagtctcagccgatggatctgtataggtatccaagccaagaccctctgtctaagctcagtctaccacccatccatacactttaccagccaaggtttggaaatagccagagttttacatctaaatacttaggttatggaaaccaaaatatgcagggagatggtttcagcagttgtaccattagaccaaatgtacatcatgtagggaaattgcctccttatcccactcatgagatggatggccacttcatgggagccacctctagattaccacccaatctgagcaatccaaacatggactataaaaatggtgaacatcattcaccttctcacataatccataactacagtgcagctccgggcatgttcaacagctctcttcatgccctgcatctccaaaacaaggagaatgacatgctttcccacacagctaatgggttatcaaagatgcttccagctcttaaccatgatagaactgcttgtgtccaaggaggcttacacaaattaagtgatgctaatggtcaggaaaagcagccattggcactagtccagggtgtggcttctggtgcagaggacaacgatgaggtctggtcagacagcgagcagagctttctggatcctgacattgggggagtggccgtggctccaactcatgggtcaattctcattgagtgtgcaaagcgtgagctgcatgccacaacccctttaaagaatcccaataggaatcaccccaccaggatctccctcgtcttttaccagcataagagcatgaatgagccaaaacatggcttggctctttgggaagccaaaatggctgaaaaagcccgtgagaaagaggaagagtgtgaaaagtatggcccagactatgtgcctcagaaatcccatggcaaaaaagtgaaacgggagcctgctgagccacatgaaacttcagagcccacttacctgcgtttcatcaagtctcttgccgaaaggaccatgtccgtgaccacagactccacagtaactacatctccatatgccttcactcgggtcacagggccttacaacagatatatatgatatcacccccttttgttggttacctcacttgaaaagaccacaaccaacctgtcagtagtatagttctcatgacgtgggcagtggggaaaggtcacagtattcatgacaaatgtggtgggaaaaacctcagctcaccagcaacaaaagaggttatcttaccatagcacttaattttcactggctcccaagtggtcacagatggcatctaggaaaagaccaaagcattctatgcaaaaagaaggtggggaagaaagtgttccgcaatttacatttttaaacactggttctattattggacgagatgatatgtaaatgtgatcccccccccccgcttacaactctacacatctgtgaccacttttaataatatcaagtttgcatagtcatggaacacaaatcaaacaagtactgtagtattacagtgacaggaatcttaaaataccatctggtgctgaatatatgatgtactgaaatactggaattatggctttttgaaatgcagtttttactgtaatcttaacttttatttatcaaaatagctacaggaaacatgaatagcaggaaaacactgaatttgtttggatgttctaagaaatggtgctaagaaaatggtgtctttaatagctaaaaatttaatgcctttatatcatcaagatgctatcagtgtactccagtgcccttgaataataggggtaccttttcattcaagtttttatcataattacctattcttacacaagcttagtttttaaaatgtggacattttaaaggcctctggattttgctcatccagtgaagtccttgtaggacaataaacgtatatatgtacatatatacacaaacatgtatatgtgcacacacatgtatatgtataaatattttaaatggtgttttagaagcactttgtctacctaagctttgacaacttgaacaatgctaaggtactgagatgtttaaaaaacaagtttactttcattttagaatgcaaagttgatttttttaaggaaacaaagaaagcttttaaaatatttttgcttttagccatgcatctgctgatgagcaattgtgtccatttttaacacagccagttaaatccaccatggggcttactggattcaagggaatacgttagtccacaaaacatgttttctggtgctcatctcacatgctatactgtaaaacagttttatacaaaattgtatgacaagttcattgctcaaaaatgtacagttttaagaattttctattaactgcaggtaataattagctgcatgctgcagactcaacaaagctagttcactgaagcctatgctattttatggatcataggctcttcagagaactgaatggcagtctgcctttgtgttgataattatgtacattgtgacgttgtcatttcttagcttaagtgtcctctttaacaagaggattgagcagactgatgcctgcataagatgaataaacagggttagttccatgtgaatctgtcagttaaaaagaaacaaaaacaggcagctggtttgctgtggtggttttaaatcattaatttgtataaagaagtgaaagagttgtatagtaaattaaattgtaaacaaaacttttttaatgcaatgctttagtattttagtactgtaaaaaaattaaatatatacatatatatatatatatatatatatatatatatgagtttgaagcagaattcacatcatgatggtgctactcagcctgctacaaatatatcataatgtgagctaagaattcattaaatgtttgagtgatgttcctacttgtcatatacctcaacactagtttggcaataggatattgaactgagagtgaaagcattgtgtaccatcatttttttccaagtccttttttttattgttaaaaaaaaaagcataccttttttcaatacttgatttcttagcaagtataacttgaacttcaacctttttgttctaaaaattcagggatatttcagctcatgctctccctatgccaacatgtcacctgtgtttatgtaaaattgttgtaggttaataaatatattctttgtcagggatttaacccttttattttgaatcccttctattttacttgtacatgtgctgatgtaactaaaactaattttgtaaatctgttggctctttttattgtaaagaaaagcattttaaaagtttgaggaatcttttgactgtttcaagcaggaaaaaaaaattacatgaaaatagaatgcactgagttgataaagggaaaaattgtaaggcaggagtttggcaagtggctgttggccagagacttacttgtaactctctaaatgaagtttttttgatcctgtaatcactgaaggtacatactccatgtggacttcccttaaacaggcaaacacctacaggtatggtgtgcaacagattgtacaattacattttggcctaaatacatttttgcttactagtatttaaaataaattcttaatcagaggaggcctttgggttttattggtcaaatctttgtaagctggcttttgtctttttaaaaaatttcttgaatttgtggttgtgtccaatttgcaaacatttccaaaaatgtttgctttgcttacaaaccacatgattttaatgttttttgtataccataatatctagccccaaacatttgattactacatgtgcattggtgattttgatcatccattcttaatatttgatttctgtgtcacctactgtcatttgttaaactgctggccaacaagaacaggaagtatagtttggggggttggggagagtttacataaggaagagaagaaattgagtggcatattgtaaatatcagatctataattgtaaatataaaacctgcctcagttagaatgaatggaaagcagatctacaatttgctaatataggaatatcaggttgactatatagccatacttgaaaatgcttctgagtggtgtcaactttacttgaatgaatttttcatcttgattgacgcacagtgatgtacagttcacttctgaagctagtggttaacttgtgtaggaaacttttgcagtttgacactaagataacttctgtgtgcatttttctatgcttttttaaaaactagtttcatttcattttcatgagatgtttggtttataagatctgaggatggttataaatactgtaagtattgtaatgttatgaatgcaggttatttgaaagctgtttattattatatcattcctgataatgctatgtgagtgtttttaataaaatttatatttatttaatgcactctaaaaaaaaaaaaaaaaaa

SEQ ID NO: 2 - protein, 2002 aa, Homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 3 - DNA, 7274 bp, Homo sapiens
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 4 - protein, 453 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 5 - DNA, 4454 bp, Homo sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattctt

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 7 - DNA, 5765 bp, Homo sapiens
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 8 - protein, 188 aa, Homo sapiens [sequence omitted]

SEQ ID NO: 9 - DNA, 4395 bp, Homo sapiens
gcagtgggctctggcggaggtcgggagaactgcagggcgaaggccgccgggggctccgcgggctgcggggggaggcacttgacaccggcccggggagaggaggggccgctgtccctgcggccagtgctggatgcggggacccagcgcagaagcagcgccaggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggcaaagaacagaaggagaccaacatcgaatccatgaaaatggagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgt

SEQ ID NO: 10 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 11 - DNA, 2591 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 12 - protein, 393 aa, Homo sapiens [sequence omitted]

SEQ ID NO: 13 - DNA, 1818 bp, Homo sapiens
tccccggcaaggcccaatggggcggcaggcccggcagccccgccccggtggtgcccgcgcggccagcgcccgccaggcccagcgttagcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactatgccgacaaaaggatcaaggtggcgaagcccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgagtccatctcaggttttgcgcacagctgcttccagtatgccatccagaagaaatggccgctgtacatgagcaccaagaacaccatactgaaagcctacgatgggcgtttcaaggacatcttccaggagatctttgacaagcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggctttggctcccttggcctgatgacgtccgtcctggtctgccctgatgggaagacgattgaggctgaggccgctcatgggaccgtcacccgccactatcgggagcaccagaagggccggcccaccagcaccaaccccatcgccagcatctttgcctggacacgtggcctggagcaccgggggaagctggatgggaaccaagacctcatcaggtttgcccagatgctggagaaggtgtgcgtggagacggtggagagtggagccatgaccaaggacctggcgggctgcattcacggcctcagcaatgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaaaaaaaaa

SEQ ID NO: 14 - protein, 452 aa, Homo sapiens
MAGYLRVVRSLCRASGSRPAWAPAALTAPTSQEQPRRHYADKRIKVAKPVVEMDGDEMTRIIWQFIKEKLILPHVDIQLKYFDLGLPNRDQTDDQVTIDSALATQKYSVAVKCATITPDEARVEEFKLKKMWKSPNGTIRNILGGTVFREPIICKNIPRLVPGWTKPITIGRHAHGDQYKATDFVADRAGTFKMVFTPKDGSGVKEWEVYNFPAGGVGMGMYNTDESISGFAHSCFQYAIQKKWPLYMSTKNTILKAYDGRFKDIFQEIFDKHYKTDFDKNKIWYEHRLIDDMVAQVLKSSGGFVWACKNYDGDVQSDILAQGFGSLGLMTSVLVCPDGKTIEAEAAHGTVTRHYREHQKGRPTSTNPIASIFAWTRGLEHRGKLDGNQDLIRFAQMLEKVCVETVESGAMTKDLAGCIHGLSNVKLNEHFLNTTDFLDTIKSNLDRALGRQ

SEQ ID NO: 15 - DNA, 2708 bp, Homo sapiens
ggcggcgcttgattgggctgggggggccaaataaaagcgatggcgattgggctgccgcgtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtccttgttgaatc

SEQ ID NO: 16 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 17 - DNA, 2402 bp, Homo sapiens
gggctgaggaggcggggcctgggaggggacaaagccgggaagaggaaaagctcggacctaccctgtggtcccgggtttctgcagagtctacttcagaagcggaggcactgggagtccggtttgggattgccaggctgtggttgtgagtctgagcttgtgagcggctgtggcgccccaactcttcgccagcatatcatcccggcaggcgataaactacattcagttgagtctgcaagactgggaggaactggggtgataagaaatctattcactgtcaaggtttattgaagtcaaaatgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtatgagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccattaaaaaaaaaa

SEQ ID NO: 18 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 19 - DNA, 1449 bp, Homo sapiens
agaaaggagtggggttgaaaagcgcttgcgcaggacggctacggtacgggggtgggagggcttcggagcacgcgcgcggaggcgggacttgggaagcgctcgcgagatcttcagggtctatatataagcgcggggagcctgcgtcctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagta

SEQ ID NO: 20 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 21 - DNA, 4772 bp, Homo sapiens
aagggcgctccgcgagcccgtctctcctcgaatgaaaggaaacaacctccggcgacagagccccgctctcaggcactgctggagaaccgagaccgacttctttctctttaccctcattggcgcttctctcctgcagtccgcctctgggccctgccgcatttcttgagacttaaagtggcattctaaaggcaatttaaaaatcatgtcaagctcagttgaacagaaaaaagggcctacaagacagcgcaaatgtggcttttgtaagtcaaatagagacaaggaatgtggacagttactaatatctgaaaaccagaaggtggcagcgcaccataagtgcatgctcttttcatctgctttggtatcatcacactctgataatgaaagtcttggtggattttctattgaagatgtccaaaaggaaattaaaagaggcacgaagctgatgtgttctttgtgccattgtcctggagcaacaattggttgtgatgtgaaaacatgtcacaggacataccactaccactgtgcattgcatgataaagctcaaatacgagagaaaccttcacaaggaatttacatggtctattgccgaaaacacaagaaaactgcacataactccgaagctgatttagaagaaagttttaatgaacatgaactggagccctcatcacctaaaagtaaaaagaaaagtcgcaaaggaaggccaagaaaaactaattttaaagggctgtcagaagataccaggtccacatcctcccatggaacagatgaaatggaaagtagttcctatagagataggtctccacacagaagcagccctagtgacaccaggcctaaatgtggattttgccatgtaggggaggaagaaaatgaagcacgaggaaaactgcatatatttaatgccaagaaggcagctgcccattataagtgcatgttgttttcttctggcacagtccagctcacaacaacatcaagagcagaatttggagactttgatattaaaactgtacttcaggagattaaacgaggaaaaagaatgaaatgtacactttgcagtcagcctggtgctactattggatgtgaaataaaagcctgtgttaagacttaccattaccactgtggagtacaagacaaagctaaatacattgaaaatatgtcacgaggaatttacaaactatactgtaaaaatcatagtggaaatgatgagagagatgaagaagatgaggaacgagagagtaaaagccgaggaaaagtagaaattgatcagcaacaactaactcagcagcaacttaatggaaactaggtatgaaagttaattatatgggatctgttgtcaggatacaatacacactgatatatacatgttgaagtaatggtatgcagtaagattttttttaatcctattatgtttagttaagagcatgttactaaaatgctgaggattttgtaggcttgcatagtagttgttttttaactagctgattttgtaaccataaaaacaagctatctgctttatcaataagtgtagttgtaatgtcaaatcaataagatattcacactcctcaataacattatattgattgtgttcccctcaagcatcatattttacatttttttatcatcttttaaacaggttcatgggacagagttagaaaactgggaatgaataagacatccataactactattcttttttcactgttttctaaaatcaaaaagggtttgtaactttttactgcccaactcttagatccttcattgaactgcctaaaaatgtcttgtttgttttatgagccctcactagaaggctgagtttgacatgttgatattacgtagctatagtttgcagtttctgggaagcaattgaaacactattaaccctgtgtatagtgtcaacagttaaagcagacgaaacgaagtaagctatatttctggactgaacaaagctctgctgtttagagcatttaagtttgtttagtggatccatactcaaaggaaagcataagcaaattttcctttacaatgcaaacatgccactggtggcagcacatggtaatttgtggatttttttttcatactcaagtaatggaggctagaaatgaagagaaccttctttttctcttgactttgacagcacatgctaaaaatctcttccaagaagtatgctaaagcttttcatttggttcctgttaaagttgttctcactgaccagcatggactcaggcaactggtaatgataagctatgagctccagtgcttttgccaaaattctcttgacagcacacctcctctctttccatgttatacaaaggacttctggcaaaatgattgagtccttcaagtttaaactaacatttggcaacagcaaattaaacgtttttaagatgtaaagaaagggttaccgcttgctaaggacttcagacaccatgtccgaaacctgttcctctcaagtgccctgttgtgtggaacacttcacatattggcgcacagtacgtaaggaggtctcatgtgctgtaggagtaaaaaagtctttgcataaaacagtatttatttttacttttgtgaccactattttagaaactttatttaatattttaatgttttcagtcattgctttggttatctataaaataggcttttgtgccctacttttcttcttgtagggcttggtggtgttttatcgattgtcagaattgtttttgaaagcctaagaacccagctttttagaaaggattttcacactggcatttctaggtagtgatattttcttccacttacctgctgaagcacatttatgcatttctttatggcaaaaccaaataactttagttgtggtctgcctgatactaccatagcacctcagtaccaaggggagggattttgactagttgaattattaagccaccacaataaagcagatttttaaataagaaataataatgttaacttgactgtacattgagatattctgcagctgatagagcagcattttaaaaatgtaacaggtggaaaattacactgtgcttaatgactgatttttttttaaactgcgagtcccttaagatcacgtttgtcaagtgtgtattcacacatttacttaaatcaagggactcaatgcttgcttttattttaaccatcttttactatttttagaaggaaactagctttagtagtgggttgccctgtatgttttctctttttgctcttaatatgccattgggtttttgtgtgtatgtgattttcaaaattcatgacttgaagtgcaaggacagatctagatgtttgtttaccaagctatgtgacttctcccaaaggatctgtactttctttccttacaacagcttgaaaatcattattttaaaatccttaaatcactgtgtctagatcattttttacattgtgtgccatagacttacccatgggacaacagagctccttcatttttggacaactactgtaatcattttttttttaggaaaaatggaaggtgccgggggtaatcatggccagtcaataattatataaaggagttcaaatactatagctgtcagttgatgaatttgttatgtagtaaaatgtatgactttgtatgggtttcttcagccctttttctgccactagcaaccagaatagcactttaccttttggttggctagataagtggctgactacctgtttttctcactgtggtgtgattggctaaacaatctcgcattaaaaattcaaatgtaaattgaattcacatgaaaaatcatgtttggttgtaaacctccaatgttttgattctctaatcatgttttcgtcacatgctgagtaaaagtgccttacaatgtaaaaattgtacagtacttatgttcccaagtagcatcatcatcttctgggtagtaattacattgtggtattaatatttagaaaaatggacctcagcagtgtatttactgtacatctcttaagtccttaactgtagttttaataagcatgacattatttgataagtatataacatttacatcatttcaaaaaatactgccgttactgtcttttatgcatattttagcctgaaattttgaagcgtcttttttctttcttttttctttttttgttttgttttttgttattgatattaaacagtgtaatctttgcaagcgtatattgaagattattctggagcatttattgccttaccagaaatgttagtaggaaatgttctttagagtagaaagatagacttgagtttctatacttttaagaagagctctttgttcctgggggaggggggcagggggtgaattttactttcatctcaagttattaaaaacccacaactgaagtaaattttatttcaagaatcagcagttttagaatttcagatagtacttgctcagaagtacatgctactcaagatattaagagataacaagatctgtagatctgctattgaatcagaatctgtgtacttgaacaaatgtgtgaatctcaaatatctcaaagcaaaagaaaaagtgttctagagtgttgttgcttttttaaaaaaagcgctgaagttagaccaaggtatacagttttgttctaacagacatttaggttaattgtaaagataaggaatgcattatgggtcaaaaatcaaacattcctctcatgttatgtatattttgttcctgttatattggctttattttcaaaattgtagtttgtagtattagtttccttttattggtattcttgcatatactattcattcaataaatgacttatgactttcataaaaaaaaaaa

SEQ ID NO: 22 - protein, 365 aa, Homo sapiens
MSSSVEQKKGPTRQRKCGFCKSNRDKECGQLLISENQKVAAHHKCMLFSSALVSSHSDNESLGGFSIEDVQKEIKRGTKLMCSLCHCPGATIGCDVKTCHRTYHYHCALHDKAQIREKPSQGIYMVYCRKHKKTAHNSEADLEESFNEHELEPSSPKSKKKSRKGRPRKTNFKGLSEDTRSTSSHGTDEMESSSYRDRSPHRSSPSDTRPKCGFCHVGEEENEARGKLHIFNAKKAAAHYKCMLFSSGTVQLTTTSRAEFGDFDIKTVLQEIKRGKRMKCTLCSQPGATIGCEIKACVKTYHYHCGVQDKAKYIENMSRGIYKLYCKNHSGNDERDEEDEERESKSRGKVEIDQQQLTQQQLNGN

SEQ ID NO: 23 - DNA, 7056 bp, Homo sapiens
cacacccacggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtattagaaaactactcggatgctccaatgacaccaaaacagattctgcaggtcatagaggcagaaggactaaaggaaatgagaagtgggacttcccctctcgcatgcctcaatgctatgctacattccaattcaagaggaggagaggggttgttttataaactgcctggccgaatcagccttttcacgctcaagaaggatgccctgcagtggtctcgccatccagctacagtggagggagaggagccagaggacacggctgatgtggagagctgtgggtctaatgaagccagcactgtgagtggtgaaaacgatgtatctcttgatgaaacatcttcgaacgcatcctgttctacagaatctcagagtcgacctctttccaatcccagggacagctacagagcttcctcacaggcgaacaaacaaaagaaaaagactggggtgatgctgcctcgagttgtcctgactcctctgaaggtaaacggggcccacgtggaatctgcatcagggttctcgggctgccacgccgatggcgagagcggcagcccgtccagcagcagcagcggctctctggccctgggcagcgctgctattcgtggccaggccgaggtcacccaggaccctgccccgctcctgagaggcttccggaagccagccacaggtcaaatgaagcgcaacagaggggaagaaatagattttgagacacctgggtccattcttgtcaacaccaacctccgtgccctgatcaactctcggaccttccatgccttaccatcacacttccagcagcagctcctcttcctcctgcctgaagtagacagacaggtggggacggatggcctgttgcgtctcagcagcagtgcactaaataacgagttttttacccatgcggctcagagctggcgggagcgcctggctgatggtgaatttactcatgagatgcaagtcaggatacgacaggaaatggagaaggaaaagaaggtggaacaatggaaagaaaagttctttgaagactactatggacagaagctgggtttgaccaaagaagagtcattgcagcagaacgtgggccaggaggaggctgaaatcaaaagtggcttgtgtgtcccaggagaatcagtgcgtatacagcgtggtccagccacccgacagcgagatgggcattttaagaaacgctctcggccagatctccgaaccagagccagaaggaatctgtacaaaaaacaggagtcagaacaagcaggggttgctaaggatgcaaaatctgtggcctcagatgttcccctctacaaggatggggaggctaagactgacccagcagggctgagcagtccccatctgccaggcacatcctctgcagcacccgacctggagggtcccgaattcccagttgagtctgtggcttctcggatccaggctgagccagacaacttggcacgtgcctctgcatctccagacagaattcctagcctgcctcaggaaactgtggatcaggaacccaaggatcagaagaggaaatcctttgagcaggcggcctctgcatcctttcccgaaaagaagccccggcttgaagatcgtcagtcctttcgtaacacaattgaaagtgttcacaccgaaaagccacagcccactaaagaggagcccaaagtcccgcccatccggattcaactttcacgtatcaaaccaccctgggtggttaaaggtcagcccacttaccagatatgcccccggatcatccccaccacggagtcctcctgccggggttggactggcgccaggaccctcgcagacattaaagcccgtgctctgcaggtccgaggggcgagaggtcaccactgccatagagaggcggccaccactgccatcggaggggggggtggcccgggtggaggtggcggcggggccaccgatgagggaggtggcagaggcagcagcagtggtgatggtggtgaggcctgtggccaccctgagcccaggggaggcccgagcacccctggaaagtgtacgtcagatctacagcgaacacaactactgccgccttatcctctaaatggggagcatacccaggccggaactgccatgtccagagctaggagagaggacctgccttctctgagaaaggaggaaagctgcctactacagagggctacagttggactcacagatgggctaggagatgcctcccaactccccgttgctcccactggggaccagccatgccaggccttgcccctactgtcctcccaaacctcagtagctgagagattagtggagcagcctcagttgcatccggatgttagaactgaatgtgagtctggcaccacttcctgggaaagtgatgatgaggagcaaggacccaccgttcctgcagacaatggtcccattccgtctctagtgggagatgatacattagagaaaggaactggccaagctcttgacagtcatcccactatgaaggatcctgtaaatgtgacccccagttccacacctgaatcctcaccgactgattgcctgcagaacagagcatttgatgacgaattagggcttggtggctcatgccctcctatgagggaaagtgatactagacaagaaaacttgaaaaccaaggctctcgtttctaacagttctttgcattggatacccatcccatcgaatgatgaggtagtgaaacagcccaaaccagaatccagagaacacataccatctgttgagccccaggttggagaggagtgggagaaagctgctcccacccctcctgcattgcctggggatttgacagctgaggagggtctagatcctcttgacagccttacttcactctggactgtgccatctcgaggaggcagtgacagcaatggcagttactgtcaacaggtggacattgaaaagctgaaaatcaacggagactctgaagcactgagtcctcacggtgagtccacggatacagcctctgactttgaaggtcacctcacggaggacagcagtgaggctgacactagagaagctgcagtgacaaagggatcttcggtggacaaggatgagaaacccaattggaaccaatctgccccactgtccaaggtgaatggtgacatgcgtctggttacaaggacagatgggatggttgctcctcagagctgggtgtctcgagtatgtgcggtccgccaaaagatcccagattccctactgctggccagtactgagtaccagccaagagccgtgtgcctgtccatgcctgggtcctcagtggaggccactaacccacttgtgatgcagttgctgcagggtagcttgcccctagagaaggttcttccaccagcccacgatgacagcatgtcagaatccccacaagtaccacttacaaaagaccagagccatggctcgctacgcatgggatctttacatggtcttggaaaaaacagtggcatggttgatggaagcagccccagttctttaagggctttgaaggagcctcttctgccagatagctgtgaaacaggcactggtcttgccaggattgaggccacccaggctcctggagcaccccaaaagaattgcaaggcagtcccaagttttgactccctccatccagtgacaaatcccattacatcctctaggaaactggaagaaatggattccaaagagcagttctcttcctttagttgtgaagatcagaaggaagtccgtgctatgtcacaggacagtaattcaaatgctgctccaggaaagagcccaggagatcttactacctcgagaacacctcgtttctcatctccaaatgtgatctcctttggtccagagcagacaggtcgggccctgggtgatcagagcaatgttacaggccaagggaagaagctttttggctctgggaatgtggctgcaacccttcagcgccccaggcctgcggacccgatgcctcttcctgctgagatccctccagtttttcccagtgggaagttgggaccaagcacaaactccatgtctggtggggtacagactccaagggaagactgggctccaaagccacatgcctttgttggcagcgtcaagaatgagaagacttttgtggggggtcctcttaaggcaaatgccgagaacaggaaagctactgggcatagtcccctggaactggtgggtcacttggaagggatgccctttgtcatggacttgcccttctggaaattaccccgagagccagggaaggggctcagtgagcctctggagccttcttctctcccctcccaactcagcatcaagcaggcattttatgggaagctttctaaactccaactgagttccaccagctttaattattcctctagctctcccacctttcccaaaggccttgctggaagtgtggtgcagctgagccacaaagcaaactttggtgcgagccacagtgcatcactttccttgcaaatgttcactgacagcagcacggtggaaagcatctcgctccagtgtgcgtgcagcctgaaagccatgatcatgtgccaaggctgcggtgcgttctgtcacgatgactgtattggaccctcaaagctctgtgtattgtgccttgtggtgagataataaattatggccatgggaaacattgtatatttagtgtgtgtattttgataatgattgatcttaaatctgtatacagaatatcattgatataatactctttaggcaggagcactcttgccttcccccaaaatttacactgctaaagccctctgtcacttggcgacccttctggtcttgctggaggggtttcctgggtataacccattgggctgcccaaggccagccagcctgagctctcctgcaagacagagcctgatgtggcacggagtggggttgcggggggtggggggactgcctgactcccagagggacttgaaactgaagcaagaaggttgcattctccaccaagggagttaacctacctgaactaagtagaaatgccagtcttccactaccccctccctgccatcttttcttctgctactttggggagttgatggccaggaaagaagccagcacagggttaaagtaactcctggcattgcccaccagggggctggtgcacctgctgacctcagggtcacagttgagtcatttgccagttgacggagcaagtttgaccttggttctgttgctgaagcaaatttggaacttttctgtctcagtgtgatccactaacccacaggatcatttggaaccttgaatagctctgcttggacaatggggttggggaatagggttgtctttcctatgaaaatgccatctgtagaccttgtgagtcagccgtccagatgtttgcaggtgaattcctctgcttgacatcctccctgtcactttggaccctatgggagtgggcatctccacgcacctgtgtatgtgaaagtcattttacatttcaaagcagtgtgtgtttcttatttttatatttttaactctttattcttggatgtataaagtgaactttttggcttctgtaagtatgctctatgcacctctaatgttttatcatgtatttatatgttgtacacagtactggctgattctgtaaatggatgtattgtacagagaacatgaacgtctcttcctaattttacatcttcagcatcattgcattaaagtggtgtaatctccttctctacatctgttgtcagagccactgagtgctgtgctgctcgacgtgagggtgaaatgattgacttgtgacctgccaggttgcccgatgccctgttgggtcaccggctggacctgctgcagcctgcagagccacagtcagcctgcccacatgccaccgagcaaacgcatcttgcttttcacatctctcctcctacagccttaatggctgcttgctgccatatgtgacaaatcaccaccaccagtgttaagtgcttctggattcatgggtgagttccctgggcagcccccaggaaggccttccagatctggctccagggtcaccacctgtcacagcaatacctgggaccatgctctcctgggactgtgaggctccttttgacgtacttttgacatcaggcaggtttgggaagaaacaaagccatgcctgctcctgcctctctcccaacatgtttccagcaagtagatgcccctgtgtgtgttttcccttgccttgtttcctgccttatatcttgtatttcgacttattacagagttgagggttcttgcttaatttagatcaagtataaaatttgtatgacttcaagtctcattttatctgaaaggtttttttctcatttaatctgatgtggcattttcgtcatctgaagcatgagtgacaagttgggaatgatgtggtgatttagaatgcagtattggccaagtccaagttgtcaacttaagcgtctgtttaccaaagaccgggaacaggggcccaaacatgtccagtcctcttcttccctctgctggaacctttggggacactcaagggtacagtttgacactgatctggtccatgaggctgcccagagaaagcactgcttctgtatgtctcttgtggtattggaacaataaacccgtacaacctgcaaaaaaaaaaaaaaaaaa

SEQ ID NO: 24 - protein, 1541 aa, Homo sapiens
MKDKQKKKKERTWAEAARLVLENYSDAPMTPKQILQVIEAEGLKEMRSGTSPLACLNAMLHSNSRGGEGLFYKLPGRISLFTLKKDALQWSRHPATVEGEEPEDTADVESCGSNEASTVSGENDVSLDETSSNASCSTESQSRPLSNPRDSYRASSQANKQKKKTGVMLPRVVLTPLKVNGAHVESASGFSGCHADGESGSPSSSSSGSLALGSAAIRGQAEVTQDPAPLLRGFRKPATGQMKRNRGEEIDFETPGSILVNTNLRALINSRTFHALPSHFQQQLLFLLPEVDRQVGTDGLLRLSSSALNNEFFTHAAQSWRERLADGEFTHEMQVRIRQEMEKEKKVEQWKEKFFEDYYGQKLGLTKEESLQQNVGQEEAEIKSGLCVPGESVRIQRGPATRQRDGHFKKRSRPDLRTRARRNLYKKQESEQAGVAKDAKSVASDVPLYKDGEAKTDPAGLSSPHLPGTSSAAPDLEGPEFPVESVASRIQAEPDNLARASASPDRIPSLPQETVDQEPKDQKRKSFEQAASASFPEKKPRLEDRQSFRNTIESVHTEKPQPTKEEPKVPPIRIQLSRIKPPWVVKGQPTYQICPRIIPTTESSCRGWTGARTLADIKARALQVRGARGHHCHREAATTAIGGGGGPGGGGGGATDEGGGRGSSSGDGGEACGHPEPRGGPSTPGKCTSDLQRTQLLPPYPLNGEHTQAGTAMSRARREDLPSLRKEESCLLQRATVGLTDGLGDASQLPVAPTGDQPCQALPLLSSQTSVAERLVEQPQLHPDVRTECESGTTSWESDDEEQGPTVPADNGPIPSLVGDDTLEKGTGQALDSHPTMKDPVNVTPSSTPESSPTDCLQNRAFDDELGLGGSCPPMRESDTRQENLKTKALVSNSSLHWIPIPSNDEVVKQPKPESREHIPSVEPQVGEEWEKAAPTPPALPGDLTAEEGLDPLDSLTSLWTVPSRGGSDSNGSYCQQVDIEKLKINGDSEALSPHGESTDTASDFEGHLTEDSSEADTREAAVTKGSSVDKDEKPNWNQSAPLSKVNGDMRLVTRTDGMVAPQSWVSRVCAVRQKIPDSLLLASTEYQPRAVCLSMPGSSVEATNPLVMQLLQGSLPLEKVLPPAHDDSMSESPQVPLTKDQSHGSLRMGSLHGLGKNSGMVDGSSPSSLRALKEPLLPDSCETGTGLARIEATQAPGAPQKNCKAVPSFDSLHPVTNPITSSRKLEEMDSKEQFSSFSCEDQKEVRAMSQDSNSNAAPGKSPGDLTTSRTPRFSSPNVISFGPEQTGRALGDQSNVTGQGKKLFGSGNVAATLQRPRPADPMPLPAEIPPVFPSGKLGPSTNSMSGGVQTPREDWAPKPHAFVGSVKNEKTFVGGPLKANAENRKATGHSPLELVGHLEGMPFVMDLPFWKLPREPGKGLSEPLEPSSLPSQLSIKQAFYGKLSKLQLSSTSFNYSSSSPTFPKGLAGSVVQLSHKANFGASHSASLSLQMFTDSSTVESISLQCACSLKAMIMCQGCGAFCHDDCIGPSKLCVLCLVVR